(19) United States
(12) Patent Application Publication
(10) Pub. No.: US 2002/0182621 A1
(43) Pub. Date: Dec. 5, 2002

(54) Methods and compositions using cap43 proteins and nucleic acids to diagnose and treat cancer and other disorders

(76) Inventors: Max Costa, Ramsey, NJ (US); Konstantin Salnikow, Waldwick, NJ (US); Herman Yee, Pomona, NY (US)

Correspondence Address: DARBY & DARBY P.C., POST OFFICE BOX 5257, NEW YORK, NY, 10150-5257, US

(21) Appl. No.: 10/057,832

(22) Filed: Jan. 25, 2002

Related U.S. Application Data
(60) Provisional application No. 60/264,268, filed on Jan. 25, 2001.

(51) Int. Cl. C12Q 1/68; G01N 33/53; G01N 33/567

(52) U.S. Cl. 435/6; 435/7.2

(57) ABSTRACT

The present invention relates to a gene, known as CAP43, and its gene product. In particular, the application demonstrates that the CAP43 gene and its gene product are expressed at elevated in certain diseased cells and tissues, such as cancer cells and tissues. The invention therefore provides novel uses for the CAP43 gene and gene product, including novel diagnostic methods for diagnosing diseases, such as cancer, by detected elevated levels of CAP43 expression. Methods are also provided for identifying diseased cells and tissue, particularly cancer cells and tissue, by identifying cells and tissues that express elevated levels of CAP43. Moreover, the invention also provides novel therapeutic methods for treating diseases, such as cancer, by targeting therapeutic compounds to cells expressing CAP43. Pharmaceutical compositions which may be used in such methods of treatment are also provided.

This application claim priority to U.S. patent application Ser. No. 60/264,268 filed on Jan. 25, 2001 and incorporated herein, by reference, in its entirety.

Work leading to the present invention was supported by Grant Nos. ES05512, ES00260 and ES10344 awarded by the National Institutes of Health/NIEHS, and by Grant No. CA16087 awarded by the National Cancer Institute. The United States government may have certain rights to this invention pursuant to the terms of those grants.

### 1. FIELD OF THE INVENTION

The present invention relates to a gene, referred to herein as CAP43, and its gene product and, in particular, to methods of using the gene and gene product to diagnose and/or treat certain diseases and disorders such as cancer. The invention also relates to kits which may be used in such diagnostic and treatment methods. The invention further relates to novel compositions, including pharmaceutical compositions, that are useful for targeting compounds (including drugs) to cells such as cancer cells and may be used in the therapeutic methods of the invention.

### 2. BACKGROUND OF THE INVENTION

Cancer is the second leading cause of death in the United States. In 1999 there were an estimated 563,100 cancer deaths and each year about 1,222,000 new cancer cases are diagnosed. Among these, solid tumor cancers such as lung, breast, prostate and colorectal cancers are the most common. Non-surgical therapy for such cancers is presently very poor. Although initial therapies for breast and ovarian cancers with taxanes have resulted in some therapeutic response by most patients, nearly all patients with ovarian cancer and many patients with breast cancer suffer some relapse. Moreover, no useful non-surgical therapies presently exist for late stage colorectal or lung cancers.

The lack of effective, non-surgical therapies for cancer is largely due to the fact that cancer is caused by the aberrant growth of an individual's own cells. Thus, the cancer cells that must be destroyed or removed in a cancer therapy are actually the patient's own, albeit abnormal, cells. Thus, almost all therapeutic reagents, such as drugs and toxins used in chemotherapy, also destroy the patient's healthy cells and tissue.

Similarly, because cancer cells are derived from healthy cells of an individual, it is typically difficult if not impossible to distinguish abnormal, cancerous cells from healthy cells and tissue of a patient. In particular, virtually all markers that are expressed by cancer cells are also expressed by other, healthy cells and tissues. Although at least one cancer-specific marker protein, known as CYP1B1, has been previously reported (see, Murray et al., Cancer Res. 1997, 57:3026-3031) other investigators have been unable to duplicate those results and even report finding the protein highly expressed in normal tissue (Mushkhelishvili et al., J Histochem Cytochem. 2001, 49:229-236). There exists, therefore, a need for specific markers that are expressed by cancer cells and can be used in diagnostic and therapeutic methods to treat cancer. In particular, there is a need for markers that can distinguish cancerous cells and tumors from normal, healthy cells and tissue. There further exist and need for markers that can be used to specifically target therapeutic compounds, including drugs and toxic compounds, to cancerous cells and tumors without affecting healthy, non-cancerous cells and tissues.

CAP43.

CAP43 (also referred to as NDRG-1) is a known gene whose expression is specifically induced by Ni²⁺ compounds such as nickel subsulfide (Ni₃S₂) and nickle dichloride (NiCl₂). See, for example, Zhou et al., Cancer Res. 1998, 58:2182-2189. Okadaic acid also induces CAP43 expression (Zhou et al., supra). CAP43 expression is also induced by calcium ionophores and this induction is attenuated by the intracellular Ca²⁺ chelators, suggesting that the primary signal for CAP43 induction is an elevation of free intracellular Ca²⁺ caused by Ni²⁺. See, Zhou et al., supra; Salnikow et al., Toxicology and Applied Pharmacology 1999, 160:127-132; and Salnikow et al. in Metals and Genetics, Chapter 8, (Sarkar, Ed.) pp. 131-144. Still other studies have revealed that CAP43 is expressed under hypoxic conditions (Salnikow et al., Cancer Res. 2000 60:38-41). Moreover, CAP43 induction by either Ni²⁺ or hypoxia requires the hypoxia-inducible transcription factor or HIF-1 (Salnikow et al., supra), a factor known to mediate transcriptional responses to hypoxia (Forsythe et al., Mol. Cell. Biol. 1996, 16:4606-4613; Semenza et al., J. Biol. Chem. 1996, 271:32529-32537).

Other researches have reported that the CAP43 gene is a differentiation marker for colon epithelium, and that its expression is lost or decreased in colon adenocarcinomas (see, van Belzen, Lab. Invest. 1997, 77:85-92). Thus, prior studies have indicated that CAP43 may be involved in the molecular mechanisms of certain cancers. However, the nature of CAP43's involvement in such cancers has not been determined. Moreover, neither the CAP43 gene nor its gene product have been heretofore identified as a possible marker for detection or identifying cancerous cells.

### 3. SUMMARY OF THE INVENTION

The present invention overcomes the above-discussed and other problems in the art by providing methods for identifying diseased cells or tissues in which the disease is one that is associated with abnormal CAP43 expression. For example, the diseased cells or tissues may be cancer cells or tissue, including but not limited to cells and tissues of a lung cancer, a colon cancer, a kidney cancer, a breast cancer, a prostate cancer, a melanoma, a lymphoma, a malignant fibrous histocytoma or any other type of cancer described herein. In other embodiments, the cells or tissues may be cells or tissues involved in inflamation such as granuloma cells or tissue. In still other embodiments, the cells or tissue may be atherosclerotic cells or tissue.

The methods of the invention involve, in one embodiment, detecting an elevated level of CAP43 nucleic acid in a cell or tissue. The CAP43 nucleic acid may be, for example, a nucleic acid that encodes a polypeptide having the amino acid sequence set forth here in SEQ ID NO: 2 (see also FIG. 1B), a nucleic acid that encodes a polypeptide having at least 70% identity, at the amino acid level, to the sequence in SEQ ID NO: 2, or a nucleic acid that hybridizes (preferably under moderately stringent or stringent conditions) to the complement of a nucleotide sequence that encodes the amino acid sequence in SEQ ID NO: 2. In particular embodiments, a CAP43 nucleic acid is a nucleic acid comprising the nucleotide sequence set forth in FIG. 1A, a nucleic acid that hybridizes (preferably under stringent or moderately stringent conditions) to the complement of the nucleotide sequence in FIG. 1A (SEQ ID NO: 1), or a nucleic acid comprising a nucleotide sequence at least 70% identical, at the nucleotide level, to the nucleotide sequence in FIG. 1A (SEQ ID NO: 1).

In another embodiment, the methods of the invention involve detecting an elevated level of a CAP43 gene product (e.g., a CAP43 polypeptide) in a cell or tissue. The CAP43 polypeptide may be, for example, a polypeptide having the amino acid sequence set forth in SEQ ID NO: 2 (see, also, FIG. 1B), a polypeptide comprising a sequence at least 70% identical, at the amino acid level, to the sequence in SEQ ID NO: 2, or a polypeptide encoded by a nucleic acid that hybridizes to the complement of a nucleic acid that encodes the amino acid sequence in SEQ ID NO: 2.

The methods of the invention are particularly well suited for diagnosing, in an individual (for example, in a human subject) a disease or disorder associated with abnormal CAP43 expression, including any of the diseases and disorders described above. Accordingly, the invention also provides methods for diagnosing such disorders by detecting, in a sample from the individual, an elevated level of a CAP43 nucleic acid. In another embodiments, the invention provides diagnostic methods which comprise detecting, in a sample from an individual, an elevated level of a CAP43 gene product (e.g., a CAP43 polypeptide).

Applicants have also discovered that CAP43 may be used, e.g., in methods of treatment, to administer compounds to diseased cells, including cells of any of the diseases and disorders described herein. Accordingly, the invention provides methods for administering a compound (for example, a drug, a pro-drug, a toxin or a cytotoxin) to a diseased cell by contacting the cell with the compound complexed to a protein (for example, an antibody) that specifically binds to a CAP43 polypeptide.

In still other embodiments, the invention provides novel compositions which may be used, e.g., to practice the above described methods. For example, the invention provides complexes which comprise (i) an antibody that specifically binds to a CAP43 polypeptide, and (ii) a therapeutic compound. Such complexes may be used, e.g., to practice the therapeutic methods of the invention. Accordingly, the invention also provides pharmaceutical compositions which comprise such complexes and a pharmaceutical acceptable carrier.

The invention further provides kits which can be used, e.g., to implement the detection and diagnostic methods of the invention. In one embodiment, the kits of the invention comprise a nucleic acid that specifically hybridizes to a CAP43 nucleic acid. In another embodiment, the kits of the invention comprise an antibody that specifically binds to a CAP43 polypeptide. In preferred embodiments, the kits of the invention also comprise instructions for their use, e.g., to detect diseased cells and tissue and/or to diagnose such diseases in individuals.

### 4. BRIEF DESCRIPTION OF THE DRAWINGS

FIGS. 1A-B show the nucleotide sequence (FIG. 1A; SEQ ID NO: 1) of a CAP43 cDNA sequence isolated from human cells and the amino acid sequence (FIG. 1B; SEQ ID NO: 2) of the polypeptide it encodes. The nucleotide sequence has also been deposited in GenBank and assigned the accession no. AF004162. The coding portion of the nucleic acid is indicated in bold-faced, underlined type. See also, FIG. 3A in Zhou et al., Cancer Research 1998, 58:2182-2189.

FIGS. 2A-B show photomicrographs of benign (FIG. 2A) and cancerous (FIG. 2B) lung tissue sections stained with a CAP43-specific antibody as described in Example 1 infra (Section 6.1).

FIGS. 3A-B show photomicrographs of benign (FIG. 3A) and malignant (FIG. 3B) breast tissue sections stained with a CAP43-specific antibody as described in Example 1 infra (Section 6.1).

FIGS. 4A-C show photomicrographs of benign (FIG. 4A) and malignant (mucinous type; FIG. 4B and tubular adenoma; FIG. 4C) colon tissue sections stained with a CAP43-specific antibody as described in Example 1 infra (Section 6.1).

FIGS. 5A-B show photomicrographs of benign (FIG. 5A) and malignant (FIG. 5B) kidney tissue sections stained with a CAP43-specific antibody as described in Example 1 infra (Section 6.1).

FIGS. 6A-B show photomicrographs of benign (FIG. 6A) and malignant (FIG. 6B) prostate tissue sections stained with CAP43-specific antibody as described in Example 1 infra (Section 6.1).

FIGS. 7A-C show photomicrographs of melanoma (FIG. 7A), malignant fibrous histiocytoma (FIG. 7B) and granuloma (FIG. 8C) tissue sections stained with CAP43-specific antibody as described in Example 1 infra (Section 6.1).

FIGS. 8A-D show photomicrographs of normal/benign liver (FIG. 8A), thyroid (FIG. 8B), placenta (FIG. 8C) and brain (FIG. 8D) tissue sections stained with CAP43-specific antibody as described in Example 1 infra (Section 6.1).

FIG. 9 shows Northern blots (top panel) indicating the level of CAP43 nucleic acid in total cellular mRNA (bottom panel) extracted from cell cultures or colon carcinoma cell lines (SW620, KM12, HCT19, HCC2998 and HCT116), lung adinocarcinoma cell lines (A549 and NCI-1460) and a melanoma cell line (M14). See, Example 2, infra (Section 6.2).

FIG. 10 shows the nucleic acid sequence of the CAP43 promoter region (SEQ ID NO: 3) immediately 5′ of the CAP43 cDNA sequence depicted in FIG. 1A (SEQ ID NO: 1). Nucleic acid residues corresponding to the consensus sequence (CGTGC) for the hypoxia-inducible transcription factor (HIF-1) are indicated in bold-faced, underlined type.

FIGS. 11A-M show photomicrographs of the following cells and/or tissues stained with a labeled CAP43-specific antibody: lung cancer tumor (FIG. 11A, 40× magnification); normal human brain (FIG. 11B, 4× magnification); malignant glioblastoma multiform (FIG. 11C, 10× magnification); malignant glioblastoma multiform (FIG. 11D, 40× magnification); malignant melanoma (FIG. 11E, 40× magnification); hyperplastic prostatic epithelial cells (FIG. 11F, 10× magnification); prostate carcinoma (FIG. 11G, 4× magnification); breast carcinoma (FIG. 11H, 40× magnification); normal kidney tissue (FIG. 11I, 40× magnification); kidney carcinoma (FIG. 11J, 40× magnification); normal (FIG. 11K, 4× magnification); colon adenoma (FIG. 11L, 10× magnification); normal skin (FIG. 11M, 40× magnification).

FIGS. 12A-E show photomicrographs of normal tissues stained with a labeled CAP43-specific antibody: normal breast tissue at 4× magnification (FIG. 12A), 10× magnification (FIG. 12B) and 40× magnification (FIG. 12C), broncheal tissue at 40× magnification (FIG. 12D) and aveolar tissue at 40× magnification (FIG. 12E).

FIGS. 13A-G show tissue staining with labeled antibody that specifically binds HIF-1α: lung, adenocarcinoma (FIG. 13A, 40× magnification); melanoma (FIG. 13B, 4× magnification); normal kidney (FIG. 13C, 40× magnification); cancerous kidney (FIG. 13D, 10× magnification); colon adenocarcinoma (FIG. 13E, 10× magnification); normal colon (FIG. 13F, 10× magnification); and malignant glioblastoma (FIG. 13G, 4× magnification).

### 5. DETAILED DESCRIPTION OF THE INVENTION

5.1. Definitions

The terms used in this specification generally have their ordinary meanings in the art, within the context of this invention and in the specific context where each term is used. Certain terms are discussed below, or elsewhere in the specification, to provide additional guidance to the practitioner in describing the compositions and methods of the invention and how to make and use them.

General Definitions.

As used herein, the term “isolated” means that the referenced material is removed from the environment in which it is normally found. Thus, an isolated biological material can be free of cellular components, i.e., components of the cells in which the material is found or produced. In the case of nucleic acid molecules, an isolated nucleic acid includes a PCR product, an isolated mRNA, a cDNA, or a restriction fragment. In another embodiment, an isolated nucleic acid is preferably excised from the chromosome in which it may be found, and more preferably is no longer joined to non-regulatory, non-coding regions, or to other genes, located upstream or downstream of the gene contained by the isolated nucleic acid molecule when found in the chromosome. In yet another embodiment, the isolated nucleic acid lacks one or more introns. Isolated nucleic acid molecules include sequences inserted into plasmids, cosmids, artificial chromosomes, and the like. Thus, in a specific embodiment, a recombinant nucleic acid is an isolated nucleic acid. An isolated protein may be associated with other proteins or nucleic acids, or both, with which it associates in the cell, or with cellular membranes if it is a membrane-associated protein. An isolated organelle, cell, or tissue is removed from the anatomical site in which it is found in an organism. An isolated material may be, but need not be, purified.

The term “purified” as used herein refers to material that has been isolated under conditions that reduce or eliminate the presence of unrelated materials, i.e., contaminants, including native materials from which the material is obtained. For example, a purified protein is preferably substantially free of other proteins or nucleic acids with which it is associated in a cell; a purified nucleic acid molecule is preferably substantially free of proteins or other unrelated nucleic acid molecules with which it can be found within a cell. As used herein, the term “substantially free” is used operationally, in the context of analytical testing of the material. Preferably, purified material substantially free of contaminants is at least 50% pure; more preferably, at least 90% pure, and more preferably still at least 99% pure. Purity can be evaluated by chromatography, gel electrophoresis, immunoassay, composition analysis, biological assay, and other methods known in the art.

Methods for purification are well-known in the art. For examples nucleic acids can be purified by precipitation, chromatography (including preparative solid phase chromatography, oligonucleotide hybridization, and triple helix chromatography), ultracentrifugation, and other means. Polypeptides and proteins can be purified by various methods including, without limitation, preparative disc-gel electrophoresis, isoelectric focusing, HPLC, reversed-phase HPLC, gel filtration, ion exchange and partition chromatography, precipitation and salting-out chromatography, extraction, and countercurrent distribution. For some purposes, it is preferable to produce the polypeptide in a recombinant system in which the protein contains an additional sequence tag that facilitates purification, such as, but not limited to, a polyhistidine sequence, or a sequence that specifically binds to an antibody, such as FLAG and GST. The polypeptide can then be purified from a crude lysate of the host cell by chromatography on an appropriate solid-phase matrix. Alternatively, antibodies produced against the protein or against peptides derived therefrom can be used as purification reagents. Cells can be purified by various techniques, including centrifugation, matrix separation (e.g., nylon wool separation), panning and other immunoselection techniques, depletion (e.g., complement depletion of contaminating cells), and cell sorting (e.g., fluorescence activated cell sorting [FACS]). Other purification methods are possible. A purified material may contain less than about 50%, preferably less than about 75%, and most preferably less than about 90%, of the cellular components with which it was originally associated. The “substantially pure” indicates the highest degree of purity which can be achieved using conventional purification techniques known in the art.

A “sample” as used herein refers to a biological material which can be tested, e.g., for the presence of CAP43 polypeptides or CAP43 nucleic acids, e.g., to identify cells that specifically express the CAP43 gene and its gene product. Such samples can be obtained from any source, including tissue, blood and blood cells, including circulating hematopoietic stem cells (for possible detection of protein or nucleic acids), plural effusions, cerebrospinal fluid (CSF), ascites fluid, and cell culture. In preferred embodiments samples are obtained, e.g., in a biopsy, from cancerous tissue (e.g., a tumor) or from tissue that is suspected of being cancerous or of containing cancer cells. In one particularly preferred embodiment samples are obtained from lung tissue.

Non-human animals include, without limitation, laboratory animals such as mice, rats, rabbits, hamsters, guinea pigs, etc.; domestic animals such as dogs and cats; and, farm animals such as sheep, goats, pigs, horses, and cows.

In preferred embodiments, the terms “about” and “approximately” shall generally mean an acceptable degree of error for the quantity measured given the nature or precision of the measurements. Typical, exemplary degrees of error are within 20 percent (%), preferably within 10%, and more preferably within 5% of a given value or range of values. Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

The term “molecule” means any distinct or distinguishable structural unit of matter comprising one or more atoms, and includes, for example, polypeptides and polynucleotides.

The term “therapeutically effective dose” refers to that amount of a compound or compositions that is sufficient to result in a desired activity. Thus, as used to describe a vaccine, a therapeutically effective dose refers to the amount of a compound or compositions (e.g., an antigen) that is sufficient to produce an effective immune response.

The phrase “pharmaceutically acceptable” refers to molecular entities and compositions that are physiologically tolerable and do not typically produce an allergic or similar untoward reaction (for example, gastric upset, dizziness and the like) when administered to an individual. Preferably, and particularly where a vaccine is used in humans, the term “pharmaceutically acceptable” may mean approved by a regulatory agency (for example, the U.S. Food and Drug Agency) or listed in a generally recognized pharmacopeia for use in animals (for example, the U.S. Pharmacopeia).

The term “carrier” refers to a diluent, adjuvant, excipient, or vehicle with which a compound is administered. Sterile water or aqueous saline solutions and aqueous dextrose and glycerol solutions are preferably employed as carriers, particularly for injectable solutions. Exemplary suitable pharmaceutical carriers are described in “Reminington's Pharmaceutical Sciences” by E. W. Martin.

Molecular Biology Definitions.

In accordance with the present invention, there may be employed conventional molecular biology, microbiology and recombinant DNA techniques within the skill of the art. Such techniques are explained fully in the literature. See, for example, Sambrook, Fitsch & Maniatis, Molecular Cloning: A Laboratory Manual, Second Edition (1989) Cold Spring Harbor Laboratory Press, Cold Spring Harbor, N.Y. (referred to herein as “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins, eds. 1984); Animal Cell Culture (R. I. Freshney, ed. 1986); Immobilized Cells and Enzymes (IRL Press, 1986); B. E. Perbal, A Practical Guide to Molecular Cloning (1984); F. M. Ausubel et al (eds.), Current Protocols in Molecular Biology, John Wiley & Sons, Inc. (1994).

The term “polymer” means any substance or compound that is composed of two or more building blocks (‘mers’) that are repetitively linked together. For example, a “dimer” is a compound in which two building blocks have been joined together; a “trimer” is a compound in which three building blocks have been joined together; etc.

The term “polynucleotide” or “nucleic acid molecule” as used herein refers to a polymeric molecule having a backbone that supports bases capable of hydrogen bonding to typical polynucleotides, wherein the polymer backbone presents the bases in a manner to permit such hydrogen bonding in a specific fashion between the polymeric molecule and a typical polynucleotide (e.g., single-stranded DNA). Such bases are typically inosine, adenosine, guanosine, cytosine, uracil and thymidine. Polymeric molecules include “double stranded” and “single stranded” DNA and RNA, as well as backbone modifications thereof (for example, methylphosphonate linkages).

Thus, a “polynucleotide” or “nucleic acid” sequence is a series of nucleotide bases (also called “nucleotides”), generally in DNA and RNA, and means any chain of two or more nucleotides. A nucleotide sequence frequently carries genetic information, including the information used by cellular machinery to make proteins and enzymes. The terms include genomic DNA, cDNA, RNA, any synthetic and genetically manipulated polynucleotide, and both sense and antisense polynucleotides. This includes single- and double-stranded molecules; i.e., DNA-DNA, DNA-RNA, and RNA-RNA hybrids as well as “protein nucleic acids” (PNA) formed by conjugating bases to an amino acid backbone. This also includes nucleic acids containing modified bases, for example, thio-uracil, thio-guanine and fluoro-uracil.

The polynucleotides herein may be flanked by natural regulatory sequences, or may be associated with heterologous sequences, including promoters, enhancers, response elements, signal sequences, polyadenylation sequences, introns, 5′- and 3′-non-coding regions and the like. The nucleic acids may also be modified by many means known in the art. Non-limiting examples of such modifications include methylation, “caps”, substitution of one or more of the naturally occurring nucleotides with an analog, and interucleotide modifications such as, for example, those with uncharged linkages (e.g., methyl phosphonates, phosphotriesters, phosphoroamidates, carbamates, etc.) and with charged linkages (e.g., phosphorothioates, phosphorodithioates, etc.). Polynucleotides may contain one or more additional covalently linked moieties, such as proteins (e.g., nucleases, toxins, antibodies, signal peptides, poly-L-lysine, etc.), intercalators (e.g., acridine, psoralen, etc.), chelators (e.g., metals, radioactive metals, iron, oxidative metals, etc.) and alkylators to name a few. The polynucleotides may be derivatized by formation of a methyl or ethyl phosphotriester or an alkyl phosphoramidite linkage. Furthermore, the polynucleotides herein may also be modified with a label capable of providing a detectable signal, either directly or indirectly. Exemplary labels include radioisotopes, fluorescent molecules, biotin and the like. Other non-limiting examples of modification which may be made are provided, below, in the description of the present invention.

A “polypeptide” is a chain of chemical building blocks called amino acids that are linked together by chemical bonds called “peptide bonds”. The term “protein” refers to polypeptides that contain the amino acid residues encoded by a gene or by a nucleic acid molecule (e.g., an mRNA or a cDNA) transcribed from that gene either directly or indirectly. Optionally, a protein may lack certain amino acid residues that are encoded by a gene or by an mRNA. For example, a gene or mRNA molecule may encode a sequence of amino acid residues on the N-terminus of a protein (i.e., a signal sequence) that is cleaved from, and therefore may not be part of, the final protein. A protein or polypeptide, including an enzyme, may be a “native” or “wild-type”, meaning that it occurs in nature; or it may be a “mutant”, “variant” or “modified”, meaning that it has been made, altered, derived, or is in some way different or changed from a native protein or from another mutant.

A “ligand” is, broadly speaking, any molecule that binds to another molecule. In preferred embodiments, the ligand is either a soluble molecule or the smaller of the two molecule or both. The other molecule is referred to as a “receptor”. In preferred embodiments, both a ligand and its receptor are molecules (preferably proteins or polypeptides) produced by cells. Preferably, a ligand is a soluble molecule and the receptor is an integral membrane protein (i.e., a protein expressed on the surface of a cell).

The binding of a ligand to its receptor is frequently a step of signal transduction within a cell. Other exemplary ligand-receptor interactions include, but are not limited to, binding of a hormone to a hormone receptor (for example, the binding of estrogen to the estrogen receptor) and the binding of a neurotransmitter to a receptor on the surface of a neuron.

“Amplification” of a polynucleotide, as used herein, denotes the use of polymerase chain reaction (PCR) to increase the concentration of a particular DNA sequence within a mixture of DNA sequences. For a description of PCR see Saiki et al., Science 1988, 239:487.

“Chemical sequencing” of DNA denotes methods such as that of Maxam and Gilbert (Maxam-Gilbert sequencing; see Maxam & Gilbert, Proc. Natl. Acad. Sci. U.S.A. 1977, 74:560), in which DNA is cleaved using individual base-specific reactions.

“Enzymatic sequencing” of DNA denotes methods such as that of Sanger (Sanger et al., Proc. Natl. Acad. Sci. U.S.A. 1977, 74:5463) and variations thereof well known in the art, in a single-stranded DNA is copied and randomly terminated using DNA polymerase.

A “gene” is a sequence of nucleotides which code for a functional “gene product”.

Generally, a gene product is a functional protein. However, a gene product can also be another type of molecule in a cell, such as an RNA (e.g., a tRNA or a rRNA). For the purposes of the present invention, a gene product also refers to an mRNA sequence which may be found in a cell. For example, measuring gene expression levels according to the invention may correspond to measuring mRNA levels. A gene may also comprise regulatory (i.e., non-coding) sequences as well as coding sequences. Exemplary regulatory sequences include promoter sequences, which determine, for example, the conditions under which the gene is expressed. The transcribed region of the gene may also include untranslated regions including introns, a 5′-untranslated region (5′-UTR) and a 3′-untranslated region (3′-UTR).

A “coding sequence” or a sequence “encoding” an expression product, such as a RNA, polypeptide, protein or enzyme, is a nucleotide sequence that, when expressed, results in the production of that RNA, polypeptide, protein or enzyme; i.e., the nucleotide sequence “encodes” that RNA or it encodes the amino acid sequence for that polypeptide, protein or enzyme.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently found, for example, by mapping with nuclease S1), as well as protein binding domains (consensus sequences) responsible for the binding of RNA polymerase.

A coding sequence is “under the control of” or is “operatively associated with” transcriptional and translational control sequences in a cell when RNA polymerase transcribes the coding sequence into RNA, which is then trans-RNA spliced (if it contains introns) and, if the sequence encodes a protein, is translated into that protein.

The term “express” and “expression” means allowing or causing the information in a gene or DNA sequence to become manifest, for example producing RNA (such as rRNA or mRNA) or a protein by activating the cellular functions involved in transcription and translation of a corresponding gene or DNA sequence. A DNA sequence is expressed by a cell to form an “expression product” such as an RNA (e.g., a mRNA or a rRNA) or a protein. The expression product itself, e.g., the resulting RNA or protein, may also said to be “expressed” by the cell.

The term “transfection” means the introduction of a foreign nucleic acid into a cell. The term “transformation” means the introduction of a “foreign” (i.e., extrinsic or extracellular) gene, DNA or RNA sequence into a host cell so that the host cell will express the introduced gene or sequence to produce a desired substance, in this invention typically an RNA coded by the introduced gene or sequence, but also a protein or an enzyme coded by the introduced gene or sequence. The introduced gene or sequence may also be called a “cloned” or “foreign” gene or sequence, may include regulatory or control sequences (e.g., start, stop, promoter, signal, secretion or other sequences used by a cell's genetic machinery). The gene or sequence may include nonfunctional sequences or sequences with no known function. A host cell that receives and expresses introduced DNA or RNA has been “transformed” and is a “transformant” or a “clone”. The DNA or RNA introduced to a host cell can come from any source, including cells of the same genus or species as the host cell or cells of a different genus or species.

The terms “vector”, “cloning vector” and “expression vector” mean the vehicle by which a DNA or RNA sequence (e.g., a foreign gene) can be introduced into a host cell so as to transform the host and promote expression (e.g., transcription and translation) of the introduced sequence. Vectors may include plasmids, phages, viruses, etc. and are discussed in greater detail below.

A “cassette” refers to a DNA coding sequence or segment of DNA that codes for an expression product that can be inserted into a vector at defined restriction sites. The cassette restriction sites are designed to ensure insertion of the cassette in the proper reading frame. Generally, foreign DNA is inserted at one or more restriction sites of the vector DNA, and then is carried by the vector into a host cell along with the transmissible vector DNA. A segment or sequence of DNA having inserted or added DNA, such as an expression vector, can also be called a “DNA construct.” A common type of vector is a “plasmid”, which generally is a self-contained molecule of double-stranded DNA, usually of bacterial origin, that can readily accept additional (foreign) DNA and which can readily introduced into a suitable host cell. A large number of vectors, including plasmid and fungal vectors, have been described for replication and/or expression in a variety of eukaryotic and prokaryotic hosts. The term “host cell” means any cell of any organism that is selected, modified, transformed, grown or used or manipulated in any way for the production of a substance by the cell. For example, a host cell may be one that is manipulated to express a particular gene, a DNA or RNA sequence, a protein or an enzyme. Host cells can further be used for screening or other assays that are described infra. Host cells may be cultured in vitro or one or more cells in a non-human animal (e.g., a transgenic animal or a transiently transfected animal).

The term “expression system” means a host cell and compatible vector under suitable conditions, e.g. for the expression of a protein coded for by foreign DNA carried by the vector and introduced to the host cell. Common expression systems include E. coli host cells and plasmid vectors, insect host cells such as Sf9, Hi5 or S2 cells and Baculovirus vectors, Drosophila cells (Schneider cells) and expression systems and mammalian host cells and vectors.

The term “heterologous” refers to a combination of elements not naturally occurring. For example, the present invention includes chimeric RNA molecules that comprise an rRNA sequence and a heterologous RNA sequence which is not part of the rRNA sequence. In this context, the heterologous RNA sequence refers to an RNA sequence that is not naturally located within the ribosomal RNA sequence. Alternatively, the heterologous RNA sequence may be naturally located within the ribosomal RNA sequence, but is found at a location in the rRNA sequence where it does not naturally occur. As another example, heterologous DNA refers to DNA that is not naturally located in the cell, or in a chromosomal site of the cell. Preferably, heterologous DNA includes a gene foreign to the cell. A heterologous expression regulatory element is a regulatory element operatively associated with a different gene that the one it is operatively associated with in nature.

The terms “mutant” and “mutation” mean any detectable change in genetic material, e.g., DNA, or any process, mechanism or result of such a change. This includes gene mutations, in which the structure (e.g., DNA sequence) of a gene is altered, any gene or DNA arising from any mutation process, and any expression product (e.g., RNA, protein or enzyme) expressed by a modified gene or DNA sequence. The term “variant” may also be used to indicate a modified or altered gene, DNA sequence, RNA, enzyme, cell, etc.; i.e., any kind of mutant. For example, the present invention relates to altered or “chimeric” RNA molecules that comprise an rRNA sequence that is altered by inserting a heterologous RNA sequence that is not naturally part of that sequence or is not naturally located at the position of that rRNA sequence. Such chimeric RNA sequences, as well as DNA and genes that encode them, are also referred to herein as “mutant” sequences.

“Sequence-conservative variants” of a polynucleotide sequence are those in which a change of one or more nucleotides in a given codon position results in no alteration in the amino acid encoded at that position.

“Function-conservative variants” of a polypeptide or polynucleotide are those in which a given amino acid residue in the polypeptide, or the amino acid residue encoded by a codon of the polynucleotide, has been changed or altered without altering the overall conformation and function of the polypeptide. For example, function-conservative variants may include, but are not limited to, replacement of an amino acid with one having similar properties (for example, polarity, hydrogen bonding potential, acidic, basic, hydrophobic, aromatic and the like). Amino acid residues with similar properties are well known in the art. For example, the amino acid residues arginine, histidine and lysine are hydrophilic, basic amino acid residues and may therefore be interchangeable. Similar, the amino acid residue isoleucine, which is a hydrophobic amino acid residue, may be replaced with leucine, methionine or valine. Such changes are expected to have little or no effect on the apparent molecular weight or isoelectric point of the polypeptide. Amino acid residues other than those indicated as conserved may also differ in a protein or enzyme so that the percent protein or amino acid sequence similarity (e.g., percent identity or homology) between any two proteins of similar function may vary and may be, for example, from 70% to 99% as determined according to an alignment scheme such as the Cluster Method, wherein similarity is based on the MEGALIGN algorithm. “Function-conservative variants” of a given polypeptide also include polypeptides that have at least 60% amino acid sequence identity to the given polypeptide as determined, e.g., by the BLAST or FASTA algorithms. Preferably, function-conservative variants of a given polypeptide have at least 75%, more preferably at least 85% and still more preferably at least 90% amino acid sequence identity to the given polypeptide and, preferably, also have the same or substantially similar properties (e.g., of molecular weight and/or isoelectric point) or functions (e.g., biological functions or activities) as the native or parent polypeptide to which it is compared. A functional conservative variant of a polypeptide may also share one or more conserved domains or sequence motifs with that polypeptide, such as the TRSRSHTSEG amino acid sequence motif or domain in CAP43 polypeptides.

The term “homologous”, in all its grammatical forms and spelling variations, refers to the relationship between two proteins that possess a “common evolutionary origin”, including proteins from superfamilies (e.g., the immunoglobulin superfamily) in the same species of organism, as well as homologous proteins from different species of organism (for example, myosin light chain polypeptide, etc.; see, Reeck et al., Cell 1987, 50:667). Such proteins (and their encoding nucleic acids) have sequence homology, as reflected by their sequence similarity, whether in terms of percent identity or by the presence of specific residues or motifs and conserved positions.

The term “sequence similarity”, in all its grammatical forms, refers to the degree of identity or correspondence between nucleic acid or amino acid sequences that may or may not share a common evolutionary origin (see, Reeck et al, supra). However, in common usage and in the instant application, the term “homologous”, when modified with an adverb such as “highly”, may refer to sequence similarity and may or may not relate to a common evolutionary origin.

In specific embodiments, two nucleic acid sequences are “substantially homologous” or “substantially similar” when at least about 80%, and more preferably at least about 90% or at least about 95% of the nucleotides match over a defined length of the nucleic acid sequences, as determined by a sequence comparison algorithm known such as BLAST, FASTA, DNA Strider, CLUSTAL, etc. An example of such a sequence is an allelic or species variant of the specific genes of the present invention. Sequences that are substantially homologous may also be identified by hybridization, e.g., in a Southern hybridization experiment under, e.g., stringent conditions as defined for that particular system.

Similarly, in particular embodiments of the invention, two amino acid sequences are “substantially homologous” or “substantially similar” when greater than 80% of the amino acid residues are identical, or when greater than about 90% of the amino acid residues are similar (i.e., are functionally identical). Preferably the similar or homologous polypeptide sequences are identified by alignment using, for example, the GCG (Genetics Computer Group, Program Manual for the GCG Package, Version 7, Madison, Wis.) pileup program, or using any of the programs and algorithms described above (e.g., BLAST, FASTA, CLUSTAL, etc.).

As used herein, the term “oligonucleotide” refers to a nucleic acid, generally of at least 10, preferably at least 15, and more preferably at least 20 nucleotides, preferably no more than 100 nucleotides, that is hybridizable to a genomic DNA molecule, a cDNA molecule, or an mRNA molecule encoding a gene, mRNA, cDNA, or other nucleic acid of interest. Oligonucleotides can be labeled, e.g., with ³²P-nucleotides or nucleotides to which a label, such as biotin or a fluorescent dye (for example, Cy3 or Cy5) has been covalently conjugated. In one embodiment, a labeled oligonucleotide can be used as a probe to detect the presence of a nucleic acid. In another embodiment, oligonucleotides (one or both of which may be labeled) can be used as PCR primers, either for cloning full length or a fragment of CAP43, or to detect the presence of nucleic acids encoding CAP43. In a further embodiment, an oligonucleotide of the invention can form a triple helix with a CAP43 DNA molecule. Generally, oligonucleotides are prepared synthetically, preferably on a nucleic acid synthesizer. Accordingly, oligonucleotides can be prepared with non-naturally occurring phosphoester analog bonds, such as thioester bonds, etc.

The present invention provides antisense nucleic acids (including ribozymes), which may be used to inhibit expression of a CAP43 gene or its gene product. An “antisense nucleic acid” is a single stranded nucleic acid molecule which, on hybridizing under cytoplasmic conditions with complementary bases in an RNA or DNA molecule, inhibits the latter's role. If the RNA is a messenger RNA transcript, the antisense nucleic acid is a countertranscript or mRNA-interfering complementary nucleic acid. As presently used, “antisense” broadly includes RNA-RNA interactions, RNA-DNA interactions, triple helix interactions, ribozymes and RNase-H mediated arrest. Antisense nucleic acid molecules can be encoded by a recombinant gene for expression in a cell (e.g., U.S. Pat. Nos. 5,814,500; 5,811,234), or alternatively they can be prepared synthetically (e.g., U.S. Pat. No. 5,780,607). Other specific examples of antisense nucleic acid molecules of the invention are provided infra.

Specific non-limiting examples of synthetic oligonucleotides envisioned for this invention include, in addition to the nucleic acid moieties described above, oligonucleotides that contain phosphorothioates, phosphotriesters, methyl phosphonates, short chain alkyl, or cycloalkyl intersugar linkages or short chain heteroatomic or heterocyclic intersugar linkages. Most preferred are those with CH₂—NH—O—CH₂, CH₂—N(CH₃)—O—CH₂, CH₂—O—N(CH₃)—CH₂, CH₂—N(CH₃)—N(CH₃)—CH₂ and O—N(CH₃)—CH₂—CH₂ backbones (where phosphodiester is O—PO₂—O—CH₂). U.S. Pat. No. 5,677,437 describes heteroaromatic olignucleoside linkages. Nitrogen linkers or groups containing nitrogen can also be used to prepare oligonucleotide mimics (U.S. Pat. Nos. 5,792,844 and 5,783,682). U.S. Pat. No. 5,637,684 describes phosphoramidate and phosphorothioamidate oligomeric compounds. Also envisioned are oligonucleotides having morpholino backbone structures (U.S. Pat. No. 5,034,506). In other embodiments, such as the peptide-nucleic acid (PNA) backbone, the phosphodiester backbone of the oligonucleotide may be replaced with a polyamide backbone, the bases being bound directly or indirectly to the aza nitrogen atoms of the polyamide backbone (Nielsen et al., Science 254:1497, 1991). Other synthetic oligonucleotides may contain substituted sugar moieties comprising one of the following at the 2′ position: OH, SH, SCH₃, F, OCN, O(CH₂)ₙNH₂ or O(CH₂)ₙCH₃ where n is from 1 to about 10; C₁ to C₁₀ lower alkyl, substituted lower alkyl, alkaryl or aralkyl; Cl; Br; CN; CF₃; OCF₃; O—; S—, or N-alkyl; O—, S—, or N-alkenyl; SOCH₃ ; SO₂CH₃; ONO₂;NO₂; N₃; NH₂; heterocycloalkyl; heterocycloalkaryl; aminoalkylamino; polyalkylamino; substitued silyl; a fluorescein moiety; an RNA cleaving group; a reporter group; an intercalator; a group for improving the pharmacokinetic properties of an oligonucleotide; or a group for improving the pharmacodynamic properties of an oligonucleotide, and other substituents having similar properties. Oligonucleotides may also have sugar mimetics such as cyclobutyls or other carbocyclics in place of the pentofuranosyl group. Nucleotide units having nucleosides other than adenosine, cytidine, guanosine, thymidine and uridine, such as inosine, may be used in an oligonucleotide molecule.

A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength (see Sambrook et al., supra). The conditions of temperature and ionic strength determine the “stringency” of the hybridization. For preliminary screening for homologous nucleic acids, low stringency hybridization conditions, corresponding to a Tₘ (melting temperature) of 55° C., can be used, e.g., 5×SSC, 0.1% SDS, 0.25% milk, and no formamide; or 30% formamide, 5×SSC, 0.5% SDS). Moderate stringency hybridization conditions correspond to a higher Tₘ, e.g., 40% formamide, with 5× or 6×SCC. High stringency hybridization conditions correspond to the highest Tₘ, e.g., 50% formamide, 5× or 6×SCC. SCC is a 0.15M NaCl, 0.015M Na-citrate. Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tₘ for hybrids of nucleic acids having those sequences. The relative stability (corresponding to higher Tₘ) of nucleic acid hybridizations decreases in the following order: RNA:RNA, DNA:RNA, DNA:DNA. For hybrids of greater than 100 nucleotides in length, equations for calculating Tₘ have been derived (see Sambrook et al., supra, 9.50-9.51). For hybridization with shorter nucleic acids, i.e., oligonucleotides, the position of mismatches becomes more important, and the length of the oligonucleotide determines its specificity (see Sambrook et al., supra, 11.7-11.8). A minimum length for a hybridizable nucleic acid is at least about 10 nucleotides; preferably at least about 15 nucleotides; and more preferably the length is at least about 20 nucleotides.

In a specific embodiment, the term “standard hybridization conditions” refers to a Tₘ of 55° C., and utilizes conditions as set forth above. In a preferred embodiment, the Tₘ is 60° C.; in a more preferred embodiment, the Tₘ is 65° C. In a specific embodiment, “thigh stringency” refers to hybridization and/or washing conditions at 68° C. in 0.2×SSC, at 42° C. in 50% formamide, 4×SSC, or under conditions that afford levels of hybridization equivalent to those observed under either of these two conditions.

Suitable hybridization conditions for oligonucleotides (e.g., for oligonucleotide probes or primers) are typically somewhat different than for full-length nucleic acids (e.g., full-length cDNA), because of the oligonucleotides' lower melting temperature. Because the melting temperature of oligonucleotides will depend on the length of the oligonucieotide sequences involved, suitable hybridization temperatures will vary depending upon the oligoncucleotide molecules used. Exemplary temperatures may be 37° C. (for 14-base oligonucleotides), 48° C. (for 17-base oligoncucleotides), 55° C. (for 20-base oligonucleotides) and 60° C. (for 23-base oligonucleotides). Exemplary suitable hybridization conditions for oligonucleotides include washing in 6×SSC/0.05% sodium pyrophosphate, or other conditions that afford equivalent levels of hybridization.

5.2. CAP43 Polypeptides

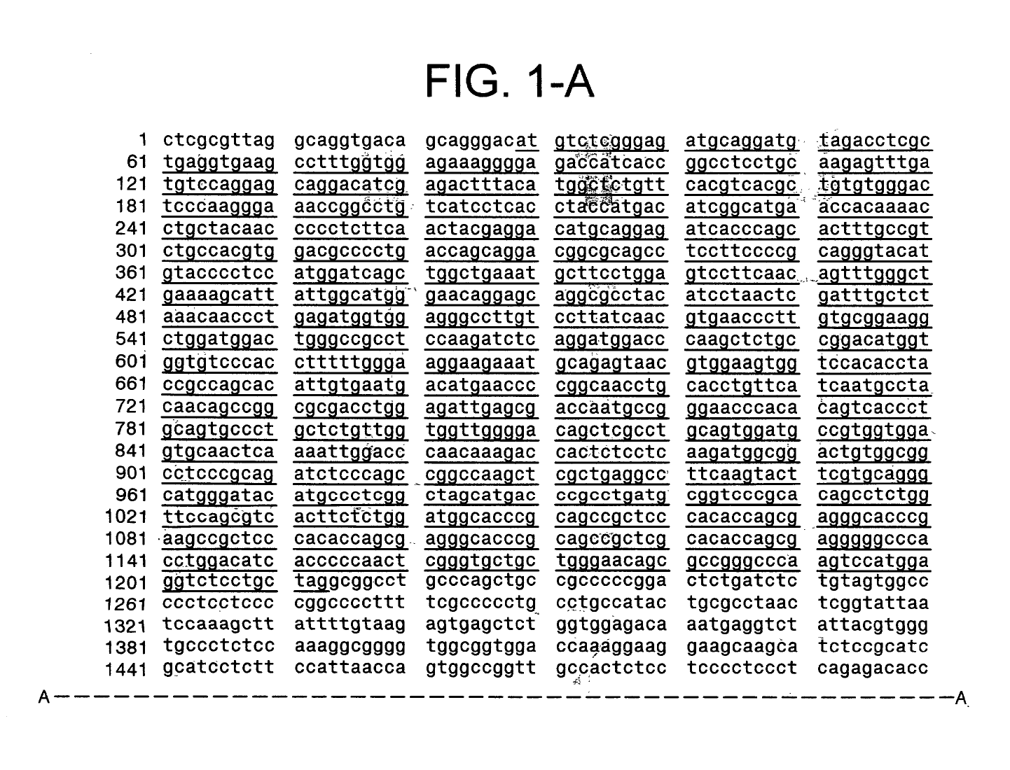

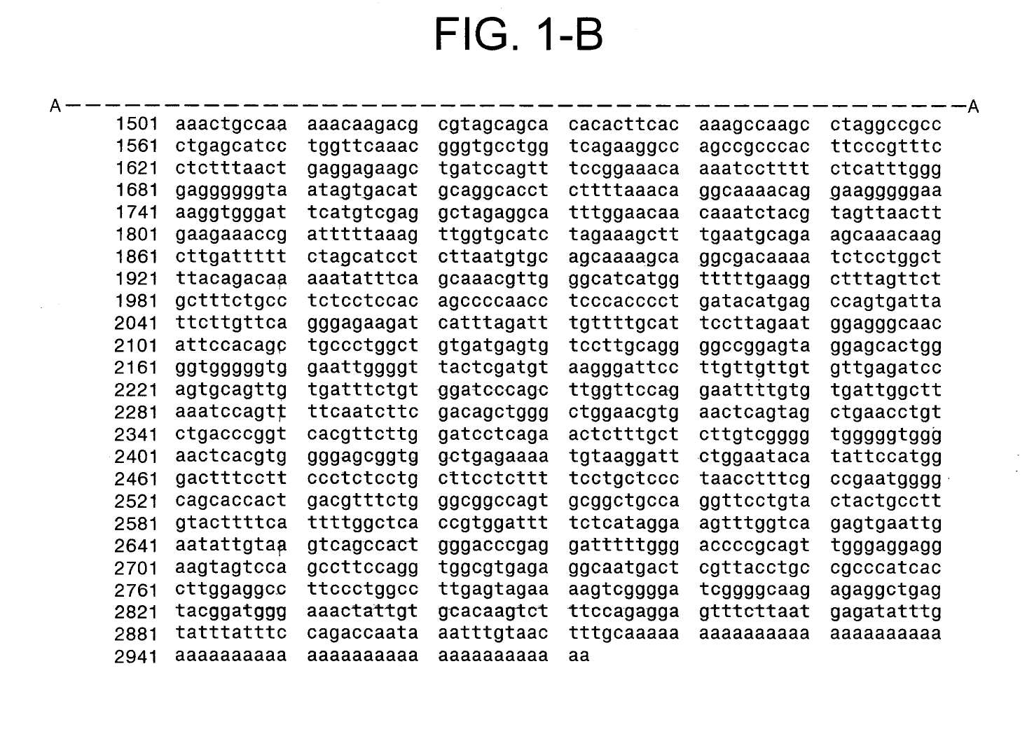

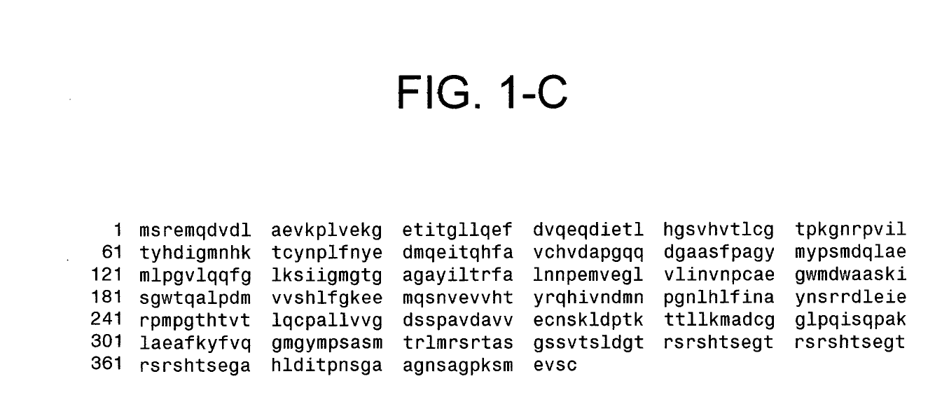

The present invention relates to a polypeptide, referred to herein as CAP43, that has been previously described. See, for example, Zhou et al., Cancer Research 1998, 43:2182-2189; Salnikow et al., Chapter 8 in Metals and Genomics (Sankar, Ed.) 1999, Kluwer Academics/Plenum Publishers, New York: 131-144; Salnikow et al., Toxicology and Applied Pharmacology 1999, 160:127-132. See also, van Belzen et al., Lab. Invest. 1997, 77:85-92. CAP43 is also referred to as NDRG-1. In one specific embodiment, a CAP43 polypeptide of the invention is derived from a human cell or has an amino acid sequence of a polypeptide derived from a human cell. For example, a human CAP43 polypeptide of the invention may comprise the amino acid sequence set forth in FIG. 1B (SEQ ID NO: 2).

CAP43 polypeptides of the invention also include polypeptides comprising an amino acid sequence of one or more epitopes or domains of a full length CAP43 polypeptide, such as epitopes or domains of the full length CAP43 polypeptide set forth in FIG. 1B (SEQ ID NO: 2). An epitope of a CAP43 polypeptide represents a site on the polypeptide against which an antibody may be produced and to which the antibody binds. Therefore, polypeptides comprising the amino acid sequence of a CAP43 epitope are useful for making antibodies to a CAP43 polypeptide. Preferably, an epitope comprises a sequence of at least 5, more preferably at least 10, 15, 20, 25, or 50 amino acid residues in length. Thus, polypeptides of the invention that comprise epitopes of a CAP43 polypeptide preferably contain an amino acid sequence corresponding to at least 5, at least 10, at least 15, at least 20, at least 25 or at least 50 amino acid residues of a full length CAP43 polypeptide sequence. For example, in certain preferred embodiments wherein the epitope is an epitope of the full length CAP43 polypeptide set forth in FIG. 1B (SEQ ID NO: 3), a CAP43 polypeptide of the invention preferably comprises an amino acid sequence corresponding to at least 5, at least 10, at least 15, at least 20, at least 25 or at least 50 amino acid residues of the sequence set forth in FIG. 1B (SEQ ID NO: 2).

The CAP43 polypeptides of the invention also include analogs and derivatives of the full length CAP43 polypeptides (e.g., of SEQ ID NO: 3). Analogs and derivatives of the CAP43 polypeptides of the invention have the same or homologous characteristics of CAP43 polypeptides set forth above.

For example, truncated forms of a CAP43 polypeptide can be provided. Such truncated forms may include a CAP43 polypeptide with a specific deletion. For instance, in certain embodiments amino acid residues corresponding to one or more domains of a full length CAP43 polypeptide may be deleted from the amino acid sequence of a CAP43 polypeptide.

In certain embodiments, a CAP43 polypeptide derivative is functionally active; i.e., it is capable of exhibiting one or more functional activities associated with a fill-length, wild-type CAP43 polypeptide of the invention.

A CAP43 chimeric or fusion polypeptide may also be prepared in which the CAP43 portion of the fusion polypeptide has one or more characteristics of the CAP43 polypeptide. Such fusion polypeptides therefore represent embodiments of the CAP43 polypeptides of this invention. Exemplary CAP43 fusion polypeptides include ones which comprise a full length, derivative or truncated CAP43 amino acid sequence, as well as fusions which comprise a fragment of a CAP43 polypeptide sequence (e.g., a fragment corresponding to an epitope or to one or more domains). Such fusion polypeptides may also comprise the amino acid sequence of a marker polypeptide; for example FLAG, a histidine tag, glutathione S-transferase (GST), or Fc portion of an IgG. In other embodiments, a CAP43 polypeptide may be expressed with (e.g., fused to) a bacterial protein such as β-galactosidase. Additionally, CAP43 fusion polypeptides may comprise amino acid sequences that increase solubility of the polypeptide, such as a thioreductase amino acid sequence or the sequence of one or more immunoglobulin proteins (e.g., IgG1 or IgG2).

CAP43 analogs or variants can also be made by altering encoding nucleic acid molecules, for example by substitutions, additions or deletions. Preferably such altered nucleic acid molecules encode functionally similar molecules (i.e., molecules that perform one or more CAP43 functions or have one or more CAP43 bioactivities). Thus, in a specific embodiment, an analog of a CAP43 polypeptide is a function-conservative variant.

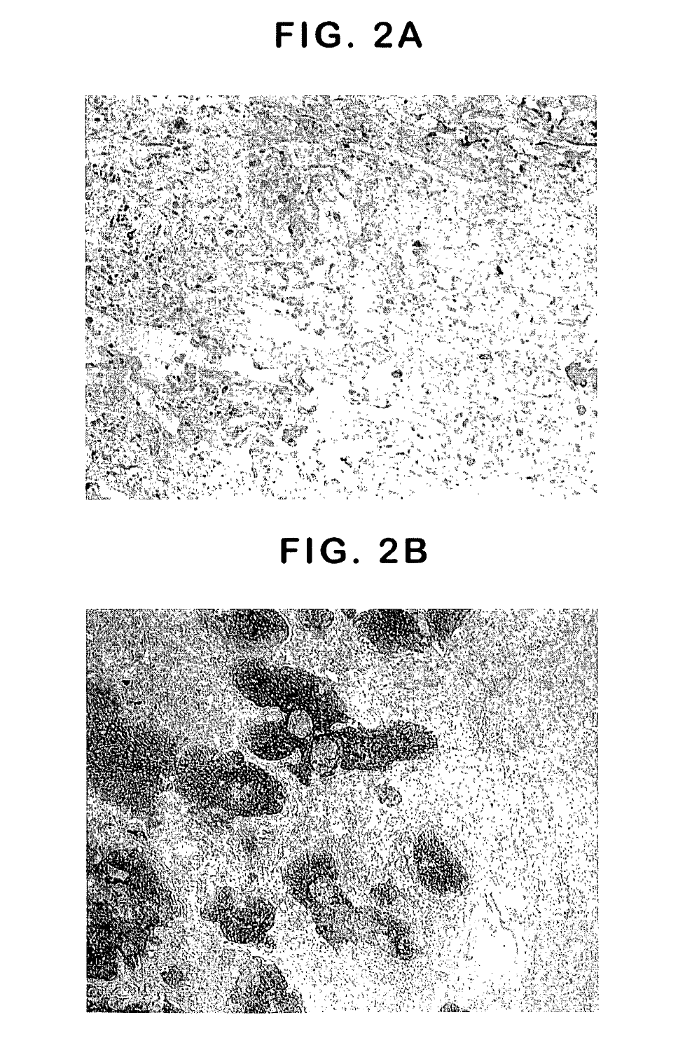

Amino acid residues, other than ones that are specifically identified herein as being conserved, may differ among variants of a protein or polypeptide. Accordingly, the percentage of protein or amino acid sequence similarity between any two CAP43 polypeptides of similar function may vary. Typically, the percentage of protein or amino acid sequence similarity between different CAP43 polypeptide variants may be from 70% to 99%, as determined according to an alignment scheme such as the Cluster Method and/or the MEGALIGN or GCG alignment algorithm. “Function-conservative variants” also include polypeptides that have greater than or at least 20%, or greater than or at least 25%, preferably greater than or at least 45%, more preferably greater than or at least 50%, still more preferably at least 75%, even more preferably at least 85%, and more preferably at least 90% or at least 95% sequence identity to a CAP43 polypeptide of the invention (e.g., the polypeptide set forth in FIG. 2 and in SEQ ID NO: 2) or to one or more fragments or domains thereof. Preferably, such function-conservative variants also have the same or similar properties, functions or bioactivities as the native polypeptide to which they are compared. It is further noted that function-conservative variants of the present invention include, not only variants of the full length CAP43 polypeptides of the invention (e.g., variants of a polypeptide comprising the sequence set forth in FIG. 1B and in SEQ ID NO: 2), but also include function-conservative variants of modified CAP43 polypeptides (e.g., truncations and deletions) and of fragments (e.g., corresponding to domains or epitopes) of full length CAP43 polypeptides.

In yet other embodiments, an analog of a CAP43 polypeptide is an allelic variant or mutant of a CAP43 polypeptide. The terms allelic variant and mutant, when used herein to describe a polypeptide, refer to a polypeptide encoded by an allelic variant or mutant gene. Thus, the allelic variant and mutant CAP43 polypeptides of the invention are polypeptides encoded by allelic variants or mutants of the CAP43 nucleic acid molecules of the present invention.

In yet other embodiments, an analog of a CAP43 polypeptide is a substantially homologous polypeptide from the same species (e.g., allelic variants) or from another species (e.g., an orthologous polypeptide). For example, CAP43 homologs and orthologs have been identified in mammals such as humans, mice, rats and hamsters. CAP43 homologs of the invention may, however, be from any species of animal, including other species of mammals (e.g., rabbit, guinea pig, dog, cat, sheep, goat, pig, horse, and cow to name a few).

While the above, exemplary variants of a CAP43 polypeptide are described in terms of the CAP43 polypeptide set forth in FIG. 1B (SEQ ID NO: 2), it is understood that variant CAP43 polypeptides of the invention include other CAP43 polypeptides having equivalent amino acid substitutions, deletions or insertions. For example, the variant CAP43 polypeptides of the invention also include fragments of the full length CAP43 polypeptide set forth in FIG. 2 (SEQ ID NO: 2) that have one or more of the amino acid substitutions, deletions or insertions described above for the full length CAP43 polypeptide.

Other homologous or variant CAP43 polypeptide sequences include allelic or species variants of the specific CAP43 polypeptide sequence set forth in FIG. 1B (SEQ ID NO: 2). Sequences that are substantially homologous can be readily identified by comparing the sequences using standard software packages available in sequence data banks, including the BLAST algorithms (e.g., BLASTP, BLASTN, BLASTX), FASTA, DNA Strider, the GCG pileup program, CLUSTAL, and other such programs which are known in the art or are described herein.

In other embodiments, variants of a CAP43 polypeptide (including analogs and homologs) are polypeptides encoded by nucleic acid molecules that hybridize to the complement of a nucleic acid molecule encoding a CAP43 polypeptide (e.g., in a Southern hybridization experiment under defined conditions). For example, in a particular embodiment analogs and/or homologs of a CAP43 polypeptide comprise amino acid sequences encoded by nucleic acid molecules that hybridize to a complement of a CAP43 nucleic acid sequence, such as the coding sequence set forth in FIG. 1A (SEQ ID NO: 1), under highly stringent hybridization conditions that comprise 50% formamide in 5× or 6×SSC. In other embodiments, the analogs and/or homologs of a CAP43 polypeptide may comprise amino acid sequences encoded by nucleic acid molecules that hybridize to a complement of a CAP43 nucleic acid sequence (e.g, the coding sequence set forth in FIG. 1A and in SEQ ID NO: 1) under moderately stringent hybridization conditions (e.g., 40% formamide with 5× or 6×SSC) or under low stringency conditions (e.g., in 5×SSC, 0.1% SDS, 0.25% milk, no formamide, 30% formamide, 5×SSC, or 0.5% SDS).

In still other embodiments, variants (including analogs, homologs and orthologs) of a CAP43 polypeptide can also be identified by isolating variant CAP43 genes, e.g., by PCR using degenerate oligonucleotide primers designed on the basis of amino acid sequences of the CAP43 polypeptide and as described below.

Derivatives of the CAP43 polypeptides of the invention further include, but are by no means limited to, phosphorylated CAP43, myristylated CAP43, methylated CAP43 and other CAP43 polypeptides that are chemically modified. CAP43 polypeptides of the invention also include labeled variants; for example, radio-labeled with iodine or phosphorous (see, e.g., EP 372707B) or other detectable molecule such as, but by no means limited to, biotin, a fluorescent dye (e.g., Cy5 or Cy3), a chelating group complexed with a metal ion, a chromophore or fluorophore, a gold colloid, a particle such as a latex bead, or attached to a water soluble polymer.

Chemical modification of a biologically active component or components of CAP43 nucleic acids or polypeptides may provide additional advantages under certain circumstances. See, for example, U.S. Pat. No. 4,179,337 issued Dec. 18, 1970 to Davis et al. Also, for a review see Abuchowski et al., in Enzymes as Drugs (J. S. Holcerberg and J. Roberts, eds. 1981), pp. 367-383. A review article describing protein modification and fusion proteins is also found in Fracis, Focus on Growth Factors 1992, 3:4-10, Mediscript: Mountview Court, Friern Barnet Lane, London N20, OLD, UK.

5.3. CAP43 Nucleic Acids

In general, a CAP43 nucleic acid molecule of the present invention comprises a nucleic acid sequence that encodes a CAP43 polypeptide as defined, supra, in Section 5.2, the complement of a nucleic acid sequence that encodes a CAP43 polypeptide, and fragments thereof. Thus, in one preferred embodiment the CAP43 nucleic acid molecules of the invention comprise a nucleotide sequence that encodes the amino acid sequence set forth in FIG. 1B (SEQ ID NO: 2), such as the particular CAP43 nucleic acid sequence set forth in FIG. 1A (SEQ ID NO: 1).

In still other embodiments, the CAP43 nucleic acid molecules of the invention comprise nucleic acid sequences that encode one or more domains of a CAP43 polypeptide.

The CAP43 nucleic acid molecules of the invention also include nucleic acids which comprise a sequence encoding one or more fragments of a CAP43 polypeptide. Such fragments include, for example, polynucleotides encoding an epitope of a CAP43 polypeptide; e.g., nucleic acids that encode a sequence of at least 5, more preferably at least, 10, 15, 20, 25 or 50 amino acid residues of a CAP43 polypeptide sequence (e.g., of the polypeptide sequence set forth in FIG. 1B and in SEQ ID NO: 2).

The CAP43 nucleic acid molecules of the invention also include nucleic acid molecules that comprise coding sequences for modified CAP43 polypeptides (e.g., having amino acid substitutions, deletions or truncations) and for variants (including allelic variants, analogs and homologs from the same or different species) of CAP43 polypeptides. In preferred embodiments, such nucleic acid molecules have at least 50%, preferably at least 75% and more preferably at least 90% sequence identity to a CAP43 coding sequence (e.g., to the CAP43 coding sequence set forth in FIG. 1 and in SEQ ID NO: 1).

In addition, the CAP43 nucleic acid molecules of the invention may also be ones that hybridize to a CAP43 nucleic acid molecule, e.g., in a Southern blot assay under defined conditions. For example, in specific embodiments a CAP43 nucleic acid molecule of the invention comprises a nucleotide sequence which hybridizes to a complement of a CAP43 nucleic acid sequence, such as the coding sequence set forth in FIG. 1 (SEQ ID NO: 1) under highly stringent hybridization conditions that comprise 50% formamide and 5× or 6×SSC. In other embodiments, the nucleic acid molecules hybridize to a complement of a CAP43 nucleic acid sequence (e.g., to the coding sequence set forth in FIG. 1 and in SEQ ID NO: 1) under moderately stringent hybridization conditions (e.g., 40% formamide with 5× or 6×SSC), or under low stringency conditions (e.g., in 5×SSC, 0.1% SDS, 0.25% milk, no formamide, 30% formamide, 5×SSC or 0.5% SDS). Alternatively, a nucleic acid molecule of the invention may hybridize, under the same defined hybridization conditions, to the complement of a fragment of a nucleotide sequence encoding a full length CAP43 polypeptide,

In other embodiments, the nucleic acid molecules of the invention comprise fragments of a full length CAP43 nucleic acid sequence. Such CAP43 nucleic acid fragments comprise a nucleotide sequence that corresponds to a sequence of at least 10 nucleotides, preferably at least 15 nucleotides and more preferably at least 20 nucleotides of a full length coding CAP43 nucleotide sequence. In specific embodiments, the fragments correspond to a portion (e.g., of at least 10, 15 or 20 nucleotides) of the CAP43 coding sequence set forth in FIG. 1 (SEQ ID NO: 1). In other preferred embodiments, the CAP43 nucleic acid fragments comprise sequences of at least 10, preferably at least 15, and more preferably at least 20 nucleotides that are complementary and/or hybridize to a full length coding CAP43 nucleic acid sequence (e.g., in the sequence set forth in FIG. 1 and in SEQ ID NO: 1) or to a fragment thereof. Suitable hybridization conditions for such oligonucleotides are described supra, and include washing in 6×SSC/0.05% sodium pyrophosphate. Because the melting temperature of oligonucleotides will depend on the length of the oligonucleotide sequence, suitable hybridization temperatures will vary depending upon the oligonucleotide molecules used. Exemplary temperatures will be 37° C. (e.g., for 14-base oligonucleotides), 48° C. (e.g., for 17-base oligonucleotides), 55° C. (e.g., for 20-base oligonucleotides) and 60° C. (e.g., for 23-base oligonucleotides).

Nucleic acid molecules comprising such fragments are useful, for example, as oligonucleotide probes and primers (e.g., PCR primers) to detect and amplify other nucleic acid molecules encoding a CAP43 polypeptide, including genes the encode variant CAP43 polypeptides such as CAP43 analogs and homologs. Oligonucleotide fragments of the invention may also be used, eg., as antisense nucleic acids, triple helix forming oligonucleotides or as ribozymes; e.g., to modulate levels of CAP43 gene expression or transcription in cells.

The nucleic acid molecules of the invention also include “chimeric” CAP43 nucleic acid molecules. Such chimeric nucleic acid molecules are polynucleotides which comprise at least one CAP43 nucleic acid sequence (which may be any of the full length or partial CAP43 nucleic acid sequences described above), and also at least one non-CAP43 nucleic acid sequence. For example, the non-CAP43 nucleic acid sequence may be a heterologous regulatory sequence (for example a promoter sequence) that is derived from another, non-CAP43 gene and is not normally associated with a naturally occurring CAP43 gene. The non-CAP43 nucleic acid sequence may also be a coding sequence of another, non-CAP43 polypeptide such as FLAG, a histidine tag, glutathione S-transferase (GST), hemaglutinin, β-galactosidase, thioreductase or an immunoglobulin domain or domains (for examples, an Fc region). In preferred embodiments, a chimeric nucleic acid molecule of the invention encodes a CAP43 fusion polypeptide of the invention.

CAP43 nucleic acid molecules of the invention, whether genomic DNA, cDNA or otherwise, can be isolated from any source including, for example, cDNA or genomic libraries derived from a cell or cell line from an organism that has a CAP43 gene. In the case of cDNA libraries, such libraries are preferably derived from a cell or cell line that expresses a CAP43 gene (for example, a cell or cell line derived from lung carcinomatous tissue such as the A549 cell line). Methods for obtaining CAP43 genes are well known in the art, as described above (see, e.g., Sambrook et al., 1989, supra).

The DNA may be obtained by standard procedures known in the art from cloned DNA (for example, from a DNA “library”), and preferably is obtained from a cDNA library prepared from tissues with high level expression of the protein (e.g., from cells or from tissue, such as lung carcinomatous cells or tissues including, for example, the A549 cell line). In one preferred embodiment, the DNA is obtained from a “subtraction” library to enrich the library for cDNAs of genes specifically expressed by a particular cell type or under certain conditions. Use of such a subtraction library may increase the likelihood of isolating cDNA for a particular gene, such as CAP43. In still other embodiments, a library may be prepared by chemical synthesis, by cDNA cloning, or by the cloning of genomic DNA or fragments thereof purified from the desired cell (See, for example, Sambrook et al., 1989, supra; Glover, D.M. ed., 1985, DNA Cloning: A Practical Approach, MRL Press, Ltd. Oxford, U.K. Vols. I and II).

In one embodiment, a cDNA library may be screened for a CAP43 nucleic acid by identifying cDNA inserts that encode a polypeptide which is homologous or substantially similar to a CAP43 polypeptide, such as the polypeptide set forth in FIG. 1B (SEQ ID NO: 2) or a fragment thereof. Similarly, a cDNA library may be screened for a CAP43 nucleic acid by identifying cDNA inserts having a nucleic acid sequence that is homologous or substantially similar to a CAP43 nucleic acid sequence, such as the nucleic acid sequence set forth in FIG. 1 (SEQ ID NO: 1) or a fragment thereof.

Clones derived from genomic DNA may contain regulatory and intron DNA regions in addition to coding regions. Clones derived from cDNA generally will not contain intron sequences. Whatever the source, the gene is preferably molecularly cloned into a suitable vector for propagation of the gene. Identification of the specific DNA fragment containing the desired CAP43 gene may be accomplished in a number of ways. For example, a portion of a CAP43 gene can be purified and labeled to prepare a labeled probe (Benton & Davis, Science 1977, 196:180; Grunstein & Hogness, Proc. Natl. Acad. Sci. U.S.A. 1975, 72:3961). Those DNA fragments with substantial homology to the probe, such as an allelic variant from another individual, will hybridize. In a specific embodiment, highest stringency hybridization conditions are used to identify a homologous CAP43 gene.

Further selection can be carried out on the basis of the properties of the CAP43 gene product, e.g., if the gene encodes a protein product having the isoelectric, electrophoretic, amino acid composition, partial or complete amino acid sequence, antibody binding activity, or ligand binding profile of a CAP43 polypeptide. Thus, the presence of the gene may be detected by assays based on the physical, chemical, immunological, or functional properties of its expressed product.

Other DNA sequences which encode substantially the same amino acid sequence as a CAP43 gene may be used in the practice of the present invention. These include but are not limited to allelic variants, species variants, sequence conservative variants, and functional variants. In particular, the nucleic acid sequences of the invention include both “function-conservative variants” and “sequence-conservative variants”. Nucleic acid substitutions may be made for example, to alter the amino acid residue encoded by a particular codon, and thereby substitute an amino acid in a CAP43 polypeptide for one with a particularly preferable property. For example, a Cysteine amino acid residue may be introduced at a potential site for disulfide bridges with another Cysteine amino acid residue. Conversly, an amino acid residue, for example a serine amino acid residue, may be substituted for a cystein amino acid residue in CAP43. Such substitutions may be useful, for example, to facilitate solubilization of a recombinant CK-2 polypeptide.

The genes encoding CAP43 derivatives and analogs of the invention can be produced by various methods known in the art. The manipulations which result in their production can occur at the gene or protein level. For example, the cloned CAP43 gene sequence can be modified by any of numerous strategies known in the art (Sambrook et al., 1989, supra). The sequence can be cleaved at appropriate sites with restriction endonuclease(s), followed by further enzymatic modification if desired, isolated, and ligated in vitro. In the production of the gene encoding a derivative or analog of CAP43, care should be taken to ensure that the modified gene remains within the same translational reading frame as the CAP43 gene, uninterrupted by translational stop signals, in the gene region where the desired activity is encoded.

Additionally, the CAP43-encoding nucleic acid sequence can be mutated in vitro or in vivo, to create and/or destroy translation, initiation, and/or termination sequences, or to create variations in coding regions and/or form new restriction endonuclease sites or destroy preexisting ones, to facilitate further in vitro modification. Modifications can also be made to introduce restriction sites and facilitate cloning the CAP43 gene into an expression vector. Any technique for mutagenesis known in the art can be used, including but not limited to, in vitro site-directed mutagenesis (Hutchinson, C., et al., J. Biol. Chem. 253:6551, 1978; Zoller and Smith, DNA 3:479-488, 1984; Oliphant et al., Gene 44:177, 1986; Hutchinson et al., Proc. Natl. Acad. Sci. U.S.A. 83:710, 1986), use of TAB″ linkers (Pharmacia), etc. PCR techniques are preferred for site directed mutagenesis (see Higuchi, 1989, “Using PCR to Engineer DNA”, in PCR Technology: Principles and Applications for DNA Amplification, H. Erlich, ed., Stockton Press, Chapter 6, pp. 61-70).

The identified and isolated gene can then be inserted into an appropriate cloning vector. A large number of vector-host systems known in the art may be used. Possible vectors include, but are not limited to, plasmids or modified viruses, but the vector system must be compatible with the host cell used. Examples of vectors include, but are not limited to, E. coli, bacteriophages such as lambda derivatives, or plasmids such as pBR322 derivatives or pUC plasmid derivatives, e.g., pGEX vectors, pmal-c, pFLAG, pKK plasmids (Clonetech), pET plasmids (Novagen, Inc., Madison, Wis.), pRSET or pREP plasmids, pcDNA (Invitrogen, Carlsbad, Calif.), or pMAL plasmids (New England Biolabs, Beverly, Mass.), etc. The insertion into a cloning vector can, for example, be accomplished by ligating the DNA fragment into a cloning vector which has complementary cohesive termini. However, if the complementary restriction sites used to fragment the DNA are not present in the cloning vector, the ends of the DNA molecules may be enzymatically modified. Alternatively, any site desired may be produced by ligating nucleotide sequences (linkers) onto the DNA termini. These ligated linkers may comprise specific chemically synthesized oligonucleotides encoding restriction endonuclease recognition sequences.

Recombinant molecules can be introduced into host cells via transformation, transfection, infection, electroporation, etc., so that many copies of the gene sequence are generated. Preferably, the cloned gene is contained on a shuttle vector plasmid, which provides for expansion in a cloning cell, e.g., E. coli, and facile purification for subsequent insertion into an appropriate expression cell line, if such is desired. For example, a shuttle vector, which is a vector that can replicate in more than one type of organism, can be prepared for replication in both E. coli and Saccharomyces cerevisiae by linking sequences from an E. coli plasmid with sequences form the yeast 2m plasmid.

5.4. Expression of CAP43 Polypeptides

A nucleotide sequence coding for CAP43, for an antigenic fragment, derivative or analog of CAP43, or for a functionally active derivative of CAP43 (including a chimeric protein) may be inserted into an appropriate expression vector, i e., a vector which contains the necessary elements for the transcription and translation of the inserted protein-coding sequence. Thus, a nucleic acid encoding a CAP43 polypeptide of the invention can be operationally associated with a promoter in an expression vector of the invention. Both cDNA and genomic sequences can be cloned and expressed under control of such regulatory sequences. Such vectors can be used to express functional or functionally inactivated CAP43 polypeptides.

The necessary transcriptional and translational signals can be provided on a recombinant expression vector.

Potential host-vector systems include but are not limited to mammalian or other vertebrate cell systems transfected with expression plasmids or infected with virus (e.g, vaccinia virus, adenovirus, adeno-associated virus, herpes virus, etc.); insect cell systems infected with virus (e.g., baculovirus); microorganisms such as yeast containing yeast vectors; or bacteria transformed with bacteriophage, DNA, plasmid DNA, or cosmid DNA. The expression elements of vectors vary in their strengths and specificities. Depending on the host-vector system utilized, any one of a number of suitable transcription and translation elements may be used.

Expression of a CAP43 protein may be controlled by any promoter/enhancer element known in the art, but these regulatory elements must be functional in the host selected for expression. Promoters which may be used to control CAP43 gene expression include, but are not limited to, cytomegalovirus (CMV) promoter (U.S. Pat. Nos. 5,385,839 and 5,168,062), the SV40 early promoter region (Benoist and Chambon, Nature 1981, 290:304-310), the promoter contained in the 3′ long terminal repeat of Rous sarcoma virus (Yamamoto, et al., Cell 1980, 22:787-797), the herpes thymidine kinase promoter (Wagner et al., Proc. Natl. Acad. Sci. U.S.A. 1981, 78:1441-1445), the regulatory sequences of the metallothionein gene (Brinster et al., Nature 1982, 296:39-42); prokaryotic expression vectors such as the b-lactamase promoter (Villa-Komaroff, et al., Proc. Natl. Acad. Sci. U.S.A. 1978, 75:3727-3731), or the tac promoter (DeBoer, et al., Proc. Natl. Acad. Sci. U.S.A. 1983, 80:21-25, 1983); see also “Useful proteins from recombinant bacteria” in Scientific American 1980, 242:74-94. Still other useful promoter elements which may be used include promoter elements from yeast or other fungi such as the Gal 4 promoter, the ADC (alcohol dehydrogenase) promoter, PGK (phosphoglycerol kinase) promoter, alkaline phosphatase promoter; and transcriptional control regions that exhibit hematopoietic tissue specificity, in particular: beta-globin gene control region which is active in myeloid cells (Mogram et al., Nature 1985, 315:338-340; Kollias et al., Cell 1986, 46:89-94), hematopoietic stem cell differentiation factor promoters, erythropoietin receptor promoter (Maouche et al., Blood 1991, 15:2557), etc.

Indeed, any type of plasmid, cosmid, YAC or viral vector may be used to prepare a recombinant nucleic acid construct which can be introduced to a cell, or to tissue, where expression of a CAP43 gene product is desired. Alternatively, wherein expression of a recombinant CAP43 gene product in a particular type of cell or tissue is desired, viral vectors that selectively infect the desired cell type or tissue type can be used.

In another embodiment, the invention provides methods for expressing CAP43 polypeptides by using a non-endogenous promoter to control expression of an endogenous CAP43 gene within a cell. An endogenous CAP43 gene within a cell is a CAP43 gene of the present invention which is ordinarily (i.e., naturally) found in the genome of tht cell. A non-endogenous promoter, however, is a promoter or other nucleotide sequence that may be used to control expression of a gene but is not ordinarily or naturally associated with the endogenous CAP43 gene. As an example, methods of homologous recombination may be employed (preferably using non-protein encoding CAP43 nucleic acid sequences of the invention) to insert an amplifiable gene or other regulatory sequence in the proximity of an endogenous CAP43 gene. The inserted sequence may then be used, e.g., to provide for higher levels of CAP43 gene expression than normally occurs in that cell, or to overcome one or more mutations in the endogenous CAP43 regulatory sequences which prevent normal levels of CAP43 gene expression. Such methods of homologous recombination are well known in the art. See, for example, International Patent Publication No. WO 91/06666, published May 16, 1991 by Skoultchi; International Patent Publication No. WO 91/099555, published Jul. 11, 1991 by Chappel; and International Patent Publication No. WO 90/14092, published Nov. 29, 1990 by Kucherlapati and Campbell.

Soluble forms of the protein can be obtained by collecting culture fluid, or solubilizing inclusion bodies, e.g., by treatment with detergent, and if desired sonication or other mechanical processes, as described above. The solubilized or soluble protein can be isolated using various techniques, such as polyacrylamide gel electrophoresis (PAGE), isoelectric focusing, 2-dimensional gel electrophoresis, chromatography (e.g., ion exchange, affinity, immunoaffinity, and sizing column chromatography), centrifugation, differential solubility, immunoprecipitation, or by any other standard technique for the purification of proteins.

A wide variety of host/expression vector combinations may be employed in expressing the DNA sequences of this invention. Useful expression vectors, for example, may consist of segments of chromosomal, non-chromosomal and synthetic DNA sequences. Suitable vectors include derivatives of SV40 and known bacterial plasmids, e.g., E. coli plasmids col El, pCR1, pBR322, pMal-C2, pET, pGEX (Smith et al., Gene 1988, 67:31-40), pCR2.1 and pcDNA 3.1+ (Invitrogen, Carlsbad, Calif.), pMB9 and their derivatives, plasmids such as RP4; phage DNAs, e.g., the numerous derivatives of phage 1, e.g., NM989, and other phage DNA, e.g., M13 and filamentous single stranded phage DNA; yeast plasmids such as the 2 m plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like.

Preferred vectors are viral vectors, such as lentiviruses, retroviruses, herpes viruses, adenoviruses, adeno-associated viruses, vaccinia virus, baculovirus, and other recombinant viruses with desirable cellular tropism. Thus, a gene encoding a functional or mutant CAP43 protein or polypeptide domain fragment thereof can be introduced in vivo, ex vivo, or in vitro using a viral vector or through direct introduction of DNA. Expression in targeted tissues can be effected by targeting the transgenic vector to specific cells, such as with a viral vector or a receptor ligand, or by using a tissue-specific promoter, or both. Targeted gene delivery is described in International Patent Publication WO 95/28494, published October 1995.

Viral vectors commonly used for in vivo or ex vivo targeting and therapy procedures are DNA-based vectors and retroviral vectors. Methods for constructing and using viral vectors are known in the art (see, e.g., Miller and Rosman, BioTechniques 1992, 7:980-990). Preferably, the viral vectors are replication defective, that is, they are unable to replicate autonomously in the target cell. In general, the genome of the replication defective viral vectors which are used within the scope of the present invention lack at least one region which is necessary for the replication of the virus in the infected cell. These regions can either be eliminated (in whole or in part), be rendered non-functional by any technique known to a person skilled in the art. These techniques include the total removal, substitution (by other sequences, in particular by the inserted nucleic acid), partial deletion or addition of one or more bases to an essential (for replication) region. Such techniques may be performed in vitro (on the isolated DNA) or in situ, using the techniques of genetic manipulation or by treatment with mutagenic agents. Preferably, the replication defective virus retains the sequences of its genome which are necessary for encapsidating the viral particles.

DNA viral vectors include an attenuated or defective DNA virus, such as but not limited to herpes simplex virus (HSV), papillomavirus, Epstein Barr virus (EBV), adenovirus, adeno-associated virus (AAV), and the like. Defective viruses, which entirely or almost entirely lack viral genes, are preferred. Defective virus is not infective after introduction into a cell. Use of defective viral vectors allows for administration to cells in a specific, localized area, without concern that the vector can infect other cells. Thus, a specific tissue can be specifically targeted. Examples of particular vectors include, but are not limited to, a defective herpes virus 1 (HSV1) vector (Kaplitt et al., Molec. Cell. Neurosci. 1991, 2:320-330), defective herpes virus vector lacking a glyco-protein L gene (Patent Publication RD 371005 A), or other defective herpes virus vectors (International Patent Publication No. WO 94/21807, published Sep. 29, 1994; International Patent Publication No. WO 92/05263, published Apr. 2, 1994); an attenuated adenovirus vector, such as the vector described by Stratford-Perricaudet et al. (J. Clin. Invest. 1992, 90:626-630; see also La Salle et al., Science 1993, 259:988-990); and a defective adeno-associated virus vector (Samulski et al., J. Virol. 1987, 61:3096-3101; Samulski et al., J. Virol. 1989, 63:3822-3828; Lebkowski et al., Mol. Cell. Biol. 1988, 8:3988-3996).

Various companies produce viral vectors commercially, including but by no means limited to Avigen, Inc. (Alameda, Calif.; AAV vectors), Cell Genesys (Foster City, Calif.; retroviral, adenoviral, AAV vectors, and lentiviral vectors), Clontech (retroviral and baculoviral vectors), Genovo, Inc. (Sharon Hill, Pa.; adenoviral and AAV vectors), Genvec (adenoviral vectors), IntroGene (Leiden, Netherlands; adenoviral vectors), Molecular Medicine (retroviral, adenoviral, AAV, and herpes viral vectors), Norgen (adenoviral vectors), Oxford BioMedica (Oxford, United Kingdom; lentiviral vectors), Transgene (Strasbourg, France; adenoviral, vaccinia, retroviral, and lentiviral vectors) and Invitrogen (Carlbad, Calif.).

In another embodiment, the vector can be introduced in vivo by lipofection, as naked DNA, or with other transfection facilitating agents (peptides, polymers, etc.). Synthetic cationic lipids can be used to prepare liposomes for in vivo transfection of a gene encoding a marker (Felgner et al., Proc. Natl. Acad. Sci. U.S.A. 1987, 84:7413-7417; Felgner and Ringold, Science 1989, 337:387-388; Mackey et al., Proc. Natl. Acad. Sci. U.S.A. 1988, 85:8027-8031; Ulmer et al., Science 1993, 259:1745-1748). Useful lipid compounds and compositions for transfer of nucleic acids are described in International Patent Publications WO 95/18863 and WO 96/17823, and in U.S. Pat. No. 5,459,127. Lipids may be chemically coupled to other molecules for the purpose of targeting (see, Mackey et al., Proc. Natl. Acad. Sci. U.S.A. 1988, 85:8027-8031). Targeted peptides, e.g., hormones or neurotransmitters, and proteins such as antibodies, or non-peptide molecules could be coupled to liposomes chemically. Other molecules are also useful for facilitating transfection of a nucleic acid in vivo, such as a cationic oligopeptide (e.g., International Patent Publication WO 95/21931), peptides derived from DNA binding proteins (e.g., International Patent Publication WO 96/25508), or a cationic polymer (e.g., International Patent Publication WO 95/21931).

It is also possible to introduce the vector in vivo as a naked DNA plasmid. Naked DNA vectors for gene therapy can be introduced into the desired host cells by methods known in the art, e.g., electroporation, microinjection, cell fusion, DEAE dextran, calcium phosphate precipitation, use of a gene gun, or use of a DNA vector transporter (see, e.g., Wu et al., J. Biol. Chem. 1992, 267:963-967; Wu and Wu, J. Biol. Chem. 1988, 263:14621-14624; Hartmut et al., Canadian Patent Application No. 2,012,311, filed Mar. 15, 1990; Williams et al., Proc. Natl. Acad. Sci. U.S.A. 1991, 88:2726-2730). Receptor-mediated DNA delivery approaches can also be used (Curiel et al., Hum. Gene Ther. 1992, 3:147-154; Wu and Wu, J. Biol. Chem. 1987, 262:4429-4432). U.S. Pat. Nos. 5,580,859 and 5,589,466 disclose delivery of exogenous DNA sequences, free of transfection facilitating agents, in a mammal. Recently, a relatively low voltage, high efficiency in vivo DNA transfer technique, termed electrotransfer, has been described (Mir et al., C.P. Acad. Sci. 1998,321:893; WO 99/01157; WO 99/01158; WO 99/01175).

Preferably, for in vivo administration, an appropriate immunosuppressive treatment is employed in conjunction with the viral vector, e.g., adenovirus vector, to avoid immuno-deactivation of the viral vector and transfected cells. For example, immunosuppressive cytokines, such as interleukin-12 (IL-12), interferon-g (IFN-γ), or anti-CD4 antibody, can be administered to block humoral or cellular immune responses to the viral vectors (see, e.g., Wilson, Nat. Med. 1995, 1:887-889). In that regard, it is advantageous to employ a viral vector that is engineered to express a minimal number of antigens.

5.5. Antibodies to CAP43

Antibodies to CAP43 are useful, inter alia, for diagnostic and therapeutic methods, as set forth below. According to the invention, CAP43 polypeptides produced, e.g., recombinantly or by chemical synthesis, and fragments or other derivatives or analogs thereof, including fusion proteins, may be used as an immunogen to generate antibodies that recognize the CAP43 polypeptide. Such antibodies include but are not limited to polyclonal, monoclonal, chimeric, single chain, Fab fragments, and an Fab expression library. Such an antibody is preferably specific for (i.e., specifically binds to) a human CAP43 polypeptide of the present invention. However, the antibody may, alternatively, be specific for a CAP43 ortholog from some other species of organism, preferably another species of mammal such as mouse, rat or hamster, to name a few. The antibody may recognize a mutant form of CAP43, or wild-type CAP43, or both.

Various procedures known in the art may be used for the production of polyclonal antibodies to CAP43 polypeptide or derivative or analog thereof. For the production of antibody, various host animals can be immunized by injection with the CAP43 polypeptide, or a derivative (e.g., fragment or fusion protein) thereof, including but not limited to rabbits, mice, rats, sheep, goats, etc. In one embodiment, the CAP43 polypeptide or fragment thereof can be conjugated to an immunogenic carrier, e.g., bovine serum albumin (BSA) or keyhole limpet hemocyanin (KLH). Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum.

For preparation of monoclonal antibodies directed toward the CAP43 polypeptide, or fragment, analog, or derivative thereof, any technique that provides for the production of antibody molecules by continuous cell lines in culture may be used. These include but are not limited to the hybridoma technique originally developed by Kohler and Milstein (Nature 1975, 256:495-497), as well as the trioma technique, the human B-cell hybridoma technique (Kozbor et al., Immunology Today 1983, 4:72; Cote et al., Proc. Natl. Acad. Sci. USA. 1983, 80:2026-2030), and the EBV-hybridoma technique to produce human monoclonal antibodies (Cole et al., in Monoclonal Antibodies and Cancer Therapy, Alan R. Liss, Inc., 1985, pp. 77-96). In an additional embodiment of the invention, monoclonal antibodies can be produced in germ-free animals (International Patent Publication No. WO 89/12690). In fact, according to the invention, techniques developed for the production of “chimeric antibodies” (Morrison et al., J. Bacteriol. 1984, 159:870; Neuberger et al., Nature 1984, 312:604-608; Takeda et al., Nature 1985, 314:452-454) may also be used. Briefly, such techniques comprise splicing the genes from an antibody molecule from a first species of organism (e.g., a mouse) that is specific for a CAP43 polypeptide together with genes from an antibody molecule of appropriate biological activity derived from a second species of organism (e.g., from a human). Such chimeric antibodies are within the scope of this invention.

Antibody fragments which contain the idiotype of the antibody molecule can be generated by known techniques. For example, such fragments include but are not limited to: the F(ab′)₂ fragment which can be produced by pepsin digestion of the antibody molecule; the Fab′ fragments which can be generated by reducing the disulfide bridges of the F(ab′)₂ fragment, and the Fab fragments which can be generated by treating the antibody molecule with papain and a reducing agent.

According to the invention, techniques described for the production of single chain antibodies (U.S. Pat. Nos. 5,476,786, 5,132,405, and 4,946,778) can be adapted to produce CAP43 polypeptide-specific single chain antibodies. An additional embodiment of the invention utilizes the techniques described for the construction of Fab expression libraries (Huse et al., Science 1989, 246:1275-1281) to allow rapid and easy identification of monoclonal Fab fragments with the desired specificity for a CK-2 polypeptide, or its derivatives, or analogs.

In the production and use of antibodies, screening for or testing with the desired antibody can be accomplished by techniques known in the art, e.g., radioimmunoassay, ELISA (enzyme-linked immunosorbant assay), “sandwich” immunoassays, immunoradiometric assays, gel diffusion precipitin reactions, immunodiffusion assays, in situ immunoassays (using colloidal gold, enzyme or radioisotope labels, for example), western blots, precipitation reactions, agglutination assays (e.g., gel agglutination assays, hemagglutination assays), complement fixation assays, immunofluorescence assays, protein A assays, and immunoelectrophoresis assays, etc. In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention. For example, to select antibodies which recognize a specific epitope of a CAP43 polypeptide, one may assay generated hybridomas for a product which binds to a CAP43 polypeptide fragment containing such epitope. For selection of an antibody specific to a CAP43 polypeptide from a particular species of animal, one can select on the basis of positive binding with CAP43 polypeptide expressed by or isolated from cells of that species of animal.

The foregoing antibodies can be used in methods known in the art relating to the localization and activity of the CAP43 polypeptide, e.g., for Western blotting, imaging CAP43 polypeptide in situ, measuring levels thereof in appropriate physiological samples, etc. using any of the detection techniques mentioned above or known in the art. Such antibodies can also be used in assays for ligand binding, e.g., as described in U.S. Pat. No. 5,679,582. Antibody binding generally occurs most readily under physiological conditions, e.g., pH of between about 7 and 8, and physiological ionic strength. The presence of a carrier protein in the buffer solutions stabilizes the assays. While there is some tolerance of perturbation of optimal conditions, e.g. increasing or decreasing ionic strength, temperature, or pH, or adding detergents or chaotropic salts, such perturbations generally decrease binding stability.

In still other embodiments, anti-CAP43 antibodies may also be used to isolate cells which express a CAP43 polypeptide by panning or related immunoadsorption techniques.

In a specific embodiment, antibodies that agonize or antagonize the activity of a CAP43 polypeptide can be generated. In particular, intracellular single chain Fv antibodies can be used to regulate (inhibit) CAP43 activity (Marasco et al., Proc. Natl. Acad. Sci. U.S.A. 1993, 90:7884-7893; Chen., Mol. Med. Today 1997, 3:160-167; Spitz et al., Anticancer Res. 1996, 16:3415-22; Indolfi et al., Nat. Med 1996, 2:634-635; Kijma et al., Pharmacol. Ther. 1995, 68:247-267). Such antibodies can be tested using the assays described infra for identifying ligands.

5.6. Applications and Uses

Described herein are various novel applications and uses for the CAP43 gene and its gene product, including novel applications and uses for CAP43 nucleic acids (described in Section 5.3, supra), for CAP43 polypeptides (described, supra, in Section 5.2) and for antibodies directed against such CAP43 polypeptides and nucleic acids (described in Section 5.5, supra). Applicants have discovered that the CAP43 gene and gene product is expressed at elevated levels in cells derived from cancerous tissue (e.g., cancer cells) than in normal, non-cancerous cells. For example, in preferred embodiments expression of a CAP43 gene and/or its gene product in cancerous tissue may be at least 10 times its level of expression in benign or healthy tissue. More preferably, expression of CAP43 gene or gene product may be at least 100 times greater in cancerous tissue than in healthy tissue. In still other embodiments, the level of CAP43 expression in cancerous tissue may be 1000 times its level of expression in benign tissue. Levels of CAP43 gene or gene product expression may be similarly elevated in other types of diseased tissue, as discussed in detail infra.

For example, the data presented in the Example, infra, shows that CAP43 polypeptide is selectively associated with tumor cells in biopsy samples. Indeed, almost no CAP43 protein is detected in normal, non-cancerous cells and tissue, including cells and tissues from the brain which express more genes than any other tissue in the body. As a result of this unexpected discovery, Applicants have determined that the CAP43 gene and its gene product may be used as a tissue specific marker to detect and/or identify cancerous cells or tissue. The CAP43 gene and its gene product may also be used to diagnose various types of cancers in an individual, e.g., by using the CAP43 gene or its gene product to detect cancerous cells or tissue in a sample such as a tissue sample (e.g., from a biopsy) from the individual. Applicants have also determined that the CAP43 gene and its gene product may be used to target compounds, including therapeutic compounds such as drugs and other pharmaceutical compositions, to cancerous cells and/or tissue. Thus, the CAP43 gene and its gene product may also be used in therapeutic regimes, e.g., to treat individuals suffering from cancer.

Without being limited to any particular theory or mechanism of action, the high level of CAP43 expression in cancer cells and tissue is believed to be induced, at least partly, in response to hypoxic conditions experienced in abnormally invading tissue such as cancer tissues and tumors. In addition, Applicants have determined that both CAP43 polypeptides and CAP43 nucleic acids (e.g., CAP43 mRNA) have extremely long half-lives both ex vivo (in cells and tissues) and in vivo (e.g., in the human body or in the body of another organism expressing CAP43).

Accordingly, the CAP43 nucleic acids and proteins described supra may actually be used in methods for the detection (e.g., in diagnostic methods) and treatment of tumors. Examples of tumors that can be detected, diagnosed and treated according to the invention include sarcomas and carcinomas such as, but not limited to: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma.

In another embodiment, CAP43 nucleic acids and polypeptides may be used to detect (e.g., to diagnose), treat or prevent dysproliferative changes (such as metaplasias and dysplasias) in epithelial tissues such as those in the cervix, esophagus, and lung. Thus, the present invention provides for detection, diagnosis and treatment of conditions known or suspected of preceding progression to neoplasia or cancer, in particular, where non-neoplastic cell growth consisting of hyperplasia, metaplasia, or most particularly, dysplasia has occurred (for review of such abnormal growth conditions, see Robbins and Angell, 1976, Basic Pathology, 2d Ed., W. B. Saunders Co., Philadelphia, pp. 68-79).

Hyperplasia is a form of controlled cell proliferation involving an increase in cell number in a tissue or organ, without significant alteration in structure or function. As but one example, endometrial hyperplasia often precedes endometrial cancer. Metaplasia is a form of controlled cell growth in which one type of adult or fully differentiated cell substitutes for another type of adult cell. Metaplasia can occur in epithelial or connective tissue cells. Atypical metaplasia involves a somewhat disorderly metaplastic epithelium. Dysplasia is frequently a forerunner of cancer, and is found mainly in the epithelia. It is the most disorderly form of non-neoplastic cell growth, involving a loss in individual cell uniformity and in the architectural orientation of cells. Dysplastic cells often have abnormally large, deeply stained nuclei, and exhibit pleomorphism. Dysplasia characteristically occurs where there exists chronic irritation or inflammation, and is often found in the cervix, respiratory passages, oral cavity, and gall bladder. For a review of such disorders, see Fishman et al., 1985, Medicine, 2d Ed., J. B. Lippincott Co., Philadelphia.

The present invention is also provides methods for the detection, diagnosis and treatment of non-malignant tumors and other disorders involving inappropriate cell or tissue growth augmented by angiogenesis by administering a therapeutically effective amount of a vector of the invention to the tissue undergoing inappropriate growth. For example, it is contemplated that the invention is useful for the treatment of arteriovenous (AV) malformations, particularly in intracranial sites. The invention may also be used to treat psoriasis, a dermatologic condition that is characterized by inflammation and vascular proliferation; and benign prostatic hypertrophy, a condition associated with inflammation and possibly vascular proliferation. The use of CAP43 nucleic acids and polypeptides for detection, diagnosis and/or treatment of other hyperproliferative disorders is also contemplated.

The CAP43 gene and its gene product may also be used to diagnose and treat other disorders. In particular, CAP43 nucleic acids and polypeptides may be used to diagnose and treat any disorder that involves or is associated with hypoxic conditions that can induce CAP43 expression in cells. For example, CAP43 nucleic acids and polypeptides may be used to identify cells, tissue and organs that have been subjected to an ischemic injury, such as from a stroke. CAP43 nucleic acids and polypeptides may also be used to identify arthersclerosis in cells, tissues or organs from an individual. Accordingly, CAP43 nucleic acids and polypeptides may also be used to diagnose conditions such as ischemia, stroke or arthersclerosis. In addition, the CAP43 nucleic acids and polypeptides can be used to treat such disorders, e.g., by using CAP43 nucleic acids or polypeptides to target therapeutic compounds to particular cells.

In other embodiments, the CAP43 gene and its gene product may be used to identify inflammatory tissue and/or cells or tissue that are involved in inflammation. For example, the CAP43 gene and its gene product may be used to identify granuloma cells and/or tissues, and may therefore be used to diagnose conditions (e.g., diseases and disorders) involving granuloma.

5.6.1. Diagnostic and Prognostic Applications

A variety of methods can be employed for diagnostic and prognostic methods using reagents such as the CAP43 nucleic acids and polypeptides described supra (including fragments, chimeras and fusions thereof) as well as antibodies directed against such CAP43 nucleic acids and polypeptides. For example, using the methods described here it is possible to detect expression of a CAP43 nucleic acid or protein in cells or tissues from an individual, such as in cells or tissues in a sample (e.g, from a biopsy) obtained or derived from an individual subject or patient. As explained above, CAP43 nucleic acids and polypeptides are expressed at elevated levels in cancerous cells and tissues including, for example, in the various types of cancer and tumor cells and tissues identified above.

Thus, using the methods described here (as well as other methods known in the art) a skilled artisan may detect elevated levels of a CAP43 nucleic acid or polypeptide in a sample of cells or tissue from an individual, and may thereby detect and/or identify cells or tissue in that sample as being cancer cells or tissue. For example, in preferred embodiments a skilled artisan may use such methods to identify cells or tissue in a sample as being a particular type of cancer cell or tissue which is known to express elevated levels of a CAP43 nucleic acid or polypeptide. Such cancer cells and tissues may be, for example, any of the particular cancer and tumor cell/tissue types described supra. In certain preferred embodiments the particular type of cancer cell or tissue identified in such methods are lung cancer cells or tissue, such as cells or tissues of bronchongeic carcinoma, lung carcinoma or small cell lung carcinoma. By using such methods to detect cancer cells or tissue in an individual, a skilled user may thereby diagnose the presence of the cancer in that individual.

In other preferred embodiments a skilled artisan may use such methods to identify dysproliferative changes (such as metaplasis and dysplasis) in cells or tissue that precedeed, and may therefore be predictive of, certain cancers. For example, in one preferred embodiment the methods may be used to detect dysproliferative changes in epithelial cells, e.g., of the lung. Accordingly, a skilled artisan may thereby diagnose an individual's predisposition to a particular type of cancer.

In preferred embodiments the methods described herein are performed using pre-packaged diagnostic kits. Such kits may comprise at least one specific CAP43 nucleic acid or a CAP43 specific antibody reagent. In preferred embodiments, a kit will also contain instructions for its use, e.g., to detect diseased cells or tissues, or to diagnose a disorder (such as cancer) associated with abnormal CAP43 expression. In preferred embodiments, such instructions may be packaged directly with the kit. In other embodiments, however, instructions may be provided separately. For example, the invention provides embodiments of kits where instructions for using the kit may be downloaded, e.g., from the internet. A kit of the invention may also comprise, preferably in separate contains, suitable buffers and other solutions to use the reagents (e.g., CAP43 specific nucleic acid or antibody) to detect CAP43. The kit and any reagent(s) contained therein may be used, for example, in a clinical setting, to diagnose patients exhibiting or suspected of having a disorder such as a type of cancer.

A sample comprising a cell of any cell type or tissue of any tissue type in which a CAP43 gene is expressed may also be used in such diagnostic methods, e.g., for detection of CAP43 gene expression or of CAP43 gene products (such as CAP43 proteins), as well as for identifying cells, particularly cancer and tumor cells, that express a CAP43 gene or a CAP43 gene product. Thus, in one embodiment, the methods described herein may be performed in situ, e.g., using cells or tissues obtained from an individual such as in a biopsy. In still other embodiments, the methods may be readily adapted without undue experimentation to detect CAP43 nucleic acid or polypeptide in vivo in tissue and/or organs of an individual. For example, tissue or organs in an individual may be stained (e.g., using labeled CAP43 specific antibodies as described in the Example, infra) so that cancerous or other diseased tissue that expresses abnormally high levels of CAP43 may be identified. Such methods may be useful, for example, in surgical procedures where it is desirable to identify and remove cancerous or other diseased tissue without removing benign, healthy tissue.

In alternative embodiments, a sample of body fluid, preferably a blood sample, may also be used in diagnostic methods of the invention. Without being limited to any particular theory or mechanism of action, both CAP43 polypeptide and CAP43 nucleic acids are believed to have an unusually long half-life. In particular, CAP43 nucleic acids and polypeptides may be released into tissues and body fluids, such as blood, after cells expressing the CAP43 nucleic acids and/or polypeptides have died or have been destroyed. Accordingly, it is expected that both CAP43 polypeptide and CAP43 nucleic acids will be present at elevated levels in body fluids, such as blood and blood plasma, from individuals having a disorder, such as cancer, where cells express CAP43 at abnormally high levels.

The methods described herein are not limited to diagnostic applications, but may also be used in prognostic applications, e.g., to monitor the progression of a disease (such as cancer) that is associated with abnormal CAP43 expression, or to monitor a therapy thereto. For example and as explained above, because CAP43 has an unusually long half-life, one skilled in the artisan may appreciate that CAP43 nucleic acids and/or polypeptides may be released into tissues and body fluids, such as blood, after cells expressing CAP43 have died or been destroyed. Accordingly, prognostic methods of the invention may comprise, in one exemplary embodiment, monitoring CAP43 nucleic acid or polypeptide levels (e.g., in the blood or other body fluids) in an individual during the course of a treatment or therapy (for example, a drug treatment or chemotherapy regime) for cancer or for another disease associated with abnormal CAP43 expression. In an effective therapy where diseased cells and tissue are killed or destroyed, such cells will release greater levels of CAP43 nucleic acid and/or polypeptide as the die. Thus, increasing CAP43 nucleic acid or polypeptide levels during the treatment are indicative of an effective treatment. Similarly, the methods of the invention may also be used to detect and identify diseased cells and tissue (for example, cancerous cells and tissue) during the course of a therapy. In such embodiments, decreasing numbers of diseased cells is generally indicative of an effective treatment. The methods of the invention may further be used, e.g., to screen candidate drugs or compounds and identify ones that may be effective, e.g., as anticancer drugs. Such methods may be performed in vivo (e.g, using an animal model) or in vitro (for example, in a cell culture assay). In one embodiment such methods may comprise contacting a candidate compound to a cell and identifying whether CAP43 expression by the cell has been inhibited. In another embodiment, a compound may be contacted to a cell or administered to an organism, and extracellular levels of CAP43 nucleic acid or polypeptide may be measured (for example, in cell culture media for cell culture assays, or in blood or other body fluid in an animal model assay). In such methods, increased extracellular levels of CAP43 are indicative of a compound that effectively kills or destroys cells (for example, cancer cells) that express abnormally high levels of CAP43.

Detection of CAP43 Nucleic Acids.

The diagnostic and prognostic methods of the invention include methods for assaying the level of CAP43 gene expression. A variety of methods known in the art may be used to detect assay levels of CAP43 nucleic acid sequences in a sample. For example, RNA from a cell type or tissue, such a tumor cell or tissue type, that is known or suspected to express the CAP43 gene may be isolated and tested utilizing hybridization or PCR techniques known in the art. The isolated cells may be, for example, cells derived from a cell culture or from an individual. The analysis of cells taken from a cell culture may be useful, e.g., to test the effect of compounds on the expression of a CAP43 gene, or alternatively, to verify that the cells are ones of a particular cell type that expresses a CAP43 gene. As an example, and not by way of limitation, diagnostic methods for the detection of CAP43 nucleic acids can involve contacting and incubating nucleic acids (including recombinant DNA molecules, cloned genes or degenerate variants thereof) obtained from a sample with one or more labeled nucleic acid reagents, such as recombinant CAP43 DNA molecules, cloned genes or degenerate variants thereof, under conditions favorable for specifically annealing or hybridizing these reagents to their complementary sequences in the sample nucleic acids. Preferably the lengths of these nucleic acid reagents are at least 15 to 30 nucleotides. After incubation, all non-annealed or non-hybridized nucleic acids are removed. The presence of nucleic acids that have hybridized, if any such molecules exist, is then detected and the level of CAP43 nucleic acid sequences to which the nucleic acid reagents have annealed may be compared to the annealing pattern or level expected from a control sample (e.g., from a sample of normal, non-cancerous cells or tissues) to determine whether CAP43 nucleic acid is expressed at an elevated level.

In a preferred embodiment of such a detection scheme, the nucleic acid from the cell type or tissue of interest may be immobilized, for example, to a solid support such as a membrane or a plastic surface (for example, on a nylon membrane, a microtiter plate or on polystyrene beads). After incubation, non-annealed, labeled CAP43 nucleic acid reagents may be easily removed and detection of the remaining, annealed, labeled CAP43 nucleic acid reagents may be accomplished using standard techniques that are well-known in the art.

Alternative diagnostic methods for the detection of CAP43 nucleic acids in patient samples or in other cell or tissue sources may involve their amplification, e.g., by PCR (see, for example, the experimental embodiment taught in U.S. Pat. No. 4,683,202) followed by detection of the amplified molecules using techniques that are well known to those of skilled in the art. The resulting level of amplified CAP43 nucleic acid may be compared to those levels that would be expected if the sample being amplified contained only normal levels of CAP43 nucleic acid, as normal cells or tissues, to determine whether elevated levels of a CAP43 nucleic acid are expressed.

In one preferred embodiment of such a detection scheme, a cDNA molecule is synthesized from an RNA molecule of interest (e.g., by reverse transcription). A sequence within the cDNA may then be used as a template for a nucleic acid amplification reaction such as PCR. Nucleic acid reagents used as synthesis intitation reagents (e.g., primers) in the reverse transcription and amplification steps of such an assay are preferably chosen from the CAP43 nucleic acid sequences described herein or are fragments thereof. Preferably, the nucleic acid reagents are at least about 9 to 30 nucleotides in length. The amplification may be performed using, e.g., radioactively labeled or fluorescently labeled nucleotides, for detection. Alternatively, enough amplified product may be made such that the product can be visualized by standard ethidium bromide or other staining methods.

CAP43 gene expression assays of the invention may also be performed in situ (i.e., directly upon tissue sections of patient tissue, which may be fixed and/or frozen), thereby eliminating the need of nucleic acid purification. CAP43 nucleic acid reagents may be used as probes or as primers for such in situ procedures (see, for example, Nuovo, PCR In Situ Hybridization: Protocols And Application, 1992, Raven Press, New York). Alternatively, if a sufficient quantity of the appropriate cells can be obtained, standard Northern analysis can be performed to determine the level of CAP43 gene express by detecting levels of CAP43 mRNA.

Detection of CAP43 Gene Products.

The diagnostic and prognostic methods of the invention also include ones that comprise detecting levels of a CAP43 protein or other CAP43 polypeptide and including functionally conserved variants and fragments thereof. For example, antibodies directed against unimpaired, wild-type or mutant CAP43 gene products or against functionally conserved variants or peptide fragments of a CAP43 gene product may be used as diagnostic and prognostic reagents, e.g., to detect various types of cancer cells and tumors. Such reagents may be used, for example, to detect abnormalities in the level of CAP43 gene product synthesis or expression, or to detect abnormalities in the structure, temporal expression or physical location of a CAP43 gene product Antibodies and immunoassay methods such as those described hereinbelow also have important in vitro applications for assessing the efficacy of treatments, e.g., for cancer. For example, antibodies, or fragments of antibodies, can be used in screens of potentially therapeutic compounds in vitro to ascertain a compound's effects on CAP43 gene expression and CAP43 polypeptide production. Compounds that may have beneficial effects on a disorder associated with abnormal CAP43 expression (e.g., any of the types of cancer identified supra) can be identified and a therapeutically effective dose for such compounds may be determined using such assays.

In vitro immunoassays can also be used to assess the efficacy of cell-based gene therapy for a cancer or other disorder associated with abnormal CAP43 expression. For example, antibodies directed against CAP43 polypeptides may be used in vitro to determine the level of CAP43 gene or polypeptide expression achieved in cells genetically engineered to produce a CAP43 polypeptide. Such methods may be used to detect intracellular CAP43 gene products, preferably using cell lysates or extracts, to detect expression of CAP43 gene products of cell surfaces, or to detect CAP43 gene products secreted into the cell culture media. Such an assessment can be used to determine the number of transformed cells necessary to achieve therapeutic efficacy in vivo, as well as optimization of the gene replacement protocol.

Generally the tissue or cell types analyzed using such methods will include ones, such as cancer and tumor cells/tissue, that are known to express a CAP43 gene product or, more preferably, expresses abnormal (typically elevated) levels of a CAP43 gene product. Protein isolation methods such as those described by Harlow & Lane (Antibodies: A Laboratory Manual, 1988, Cold Spring Harbor Laboratory Press, Cold Spring Harbor, N.Y.) may be employed. The isolated cells may be cells derived from cell culture or from an individual (e.g., a biopsy sample from a patient suspected of a type of cancer or other disorder associated with abnormal levels of CAP43 expression, or suspected of having a propensity for such a cancer or other disorder).

As one example, antibodies or fragments of antibodies may be used to detect the presence of a CAP43 gene product, a variant of a CAP43 gene product or fragments thereof, for example, by immunofluorescence techniques employing a fluorescently labeled antibody coupled with light microscopic, flow cytometric or fluorimetric detection methods.

In particularly preferred embodiments, antibodies or fragments thereof may also be employed histologically, for example in immunofluorescence or immunoelectron microscopy techniques, for in situ detection of a CAP43 gene product. In situ detection may be accomplished by removing a histological specimen (e.g., a tissue sample) from a patient and applying thereto a labeled antibody of the present invention or a fragment of such an antibody. The antibody or antibody fragment is preferably applied by overlaying the labeled antibody or antibody fragment onto a biological sample. Through the use of such a procedure, it is possible to detect, not only the presence of a CAP43 gene product, but also the gene product's distribution in the examined tissue. A wide variety of histological methods that are well known in the art (for example, staining procedures) can be readily modified by those skilled in the art without undue experimentation to achieve such in situ detection.

Immunoassays for CAP43 gene products will typically comprise incubating a biological sample (for example, a biological fluid, a tissue extract, freshly harvested cells or cell lysates) in the presence of a detectably labeled antibody that is capable of specifically binding a CAP43 gene product (including, for example, a functionally conserved variant or a peptide fragment thereof). The bound antibody may then be detected by any of a number of techniques well known in the art.

5.6.2. Screening Assays

Using screening assays such as those described hereinbelow, it is also possible to identify compounds that bind to or otherwise interact with a CAP43 gene product, including intracellular compounds (for example, proteins or portions of proteins) that interact with a CAP43 gene product, natural and synthetic ligands (or receptors) for a CAP43 gene product, compounds that interfere with the interaction of a CAP43 gene product (for example, compounds that interfere with specific binding of a CAP43 gene product to a receptor or intracellular compound), and compounds that modulate the activity of a CAP43 gene (for example, by modulating the level of CAP43 gene expression) or the activity (for example, the bioactivity) of a CAP43 polypeptide or other CAP43 gene products.

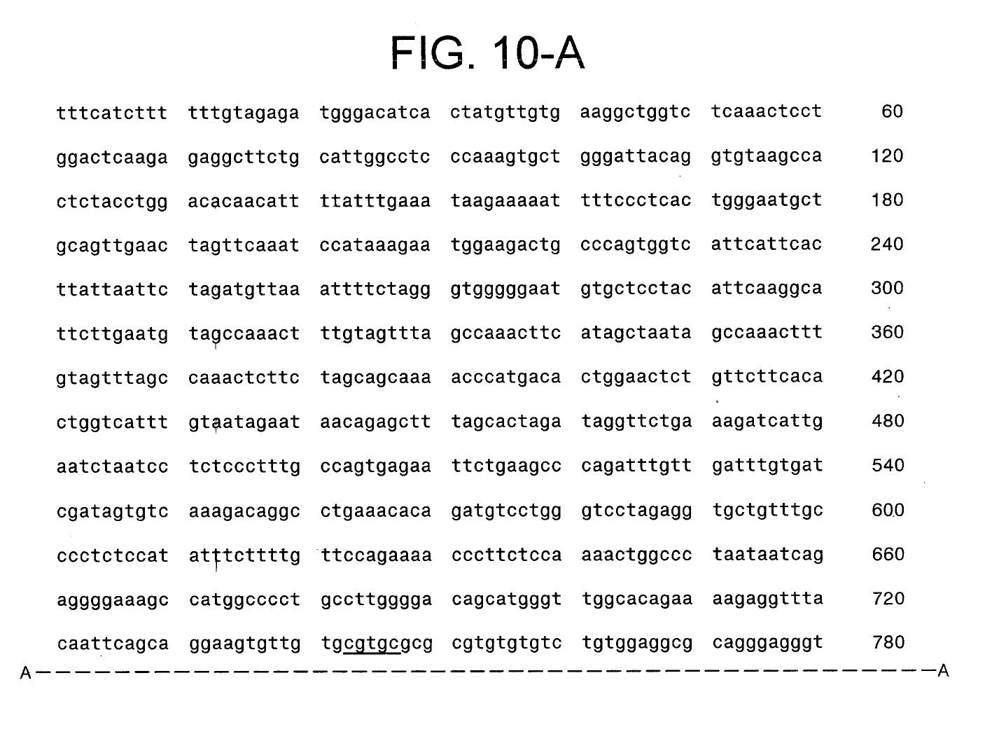

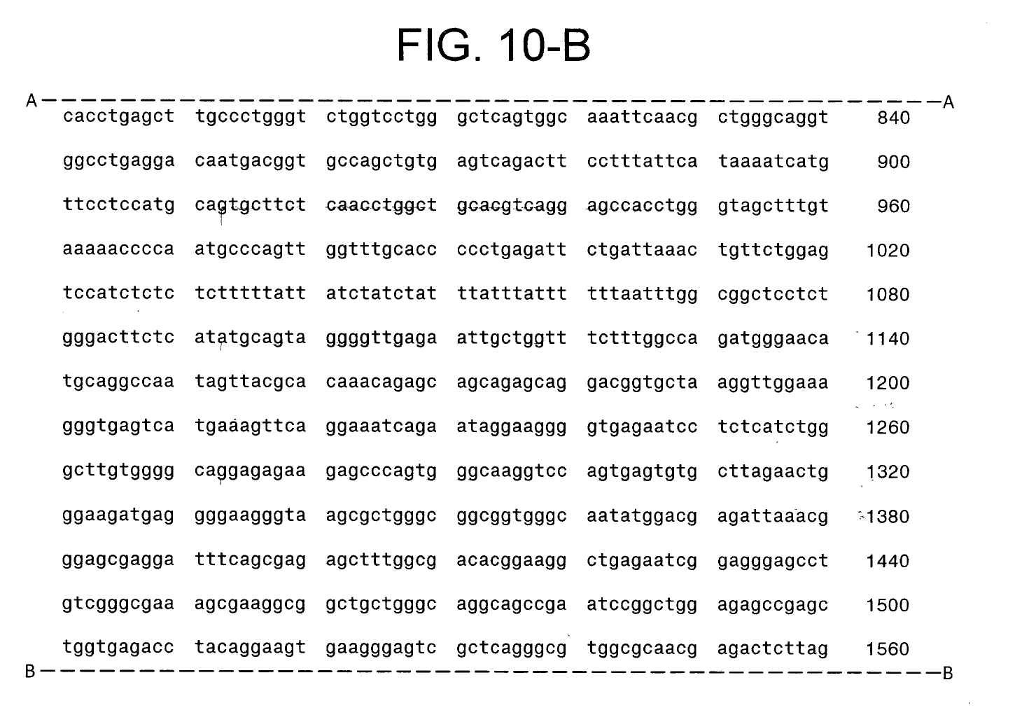

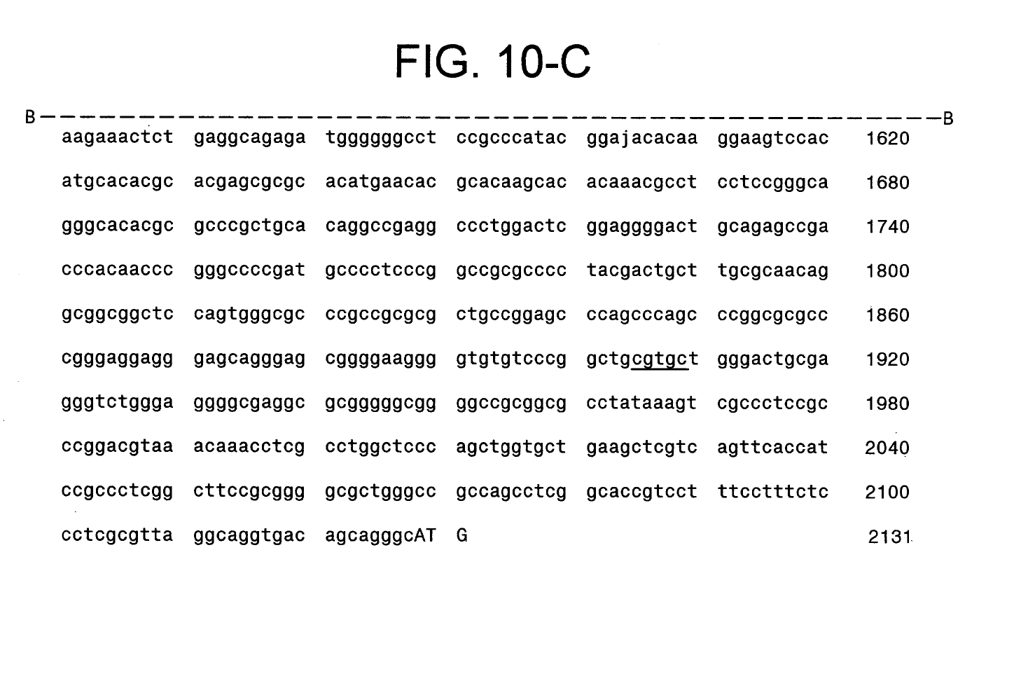

For example and without being bound by any particular theory or mechanism of action, expression of CAP43 nucleic acids and polypeptides of the present invention is believed to being induced or enhanced by hypoxic conditions and, consequently, these nucleic acids and polypeptides are thought to interact with other cellular constituents (e.g., other proteins or nucleic acids) that mediate cellular responses to hypoxia. For example, the human CAP43 genomic sequence is believed to contain at least hypoxia responsive element or HRE that interacts with a transcription factor (for example, the hypoxia-inducible transcription factor or HIF-1; see, Forsythe et al., Mol. Cell. Biol. 1996, 16:4604-4613; Semenza et al., J. Biol. Chem. 1996, 271:32529-32537; also, for a review see Semenza, Annu. Rev. Cell. Dev. Biol. 1999, 15:551-578) to modulate expression of the CAP43 gene. Moreover, CAP43 nucleic acids (for example, CAP43 mRNA) may contain one or more other HREs that interact with other cellular constituents (for example, with other proteins or nucleic acids) to modulate, e.g., translation of CAP43 mRNA and/or CAP43 polypeptide expression. For example, the consensus sequence for the HIF-1 binding site (cgtgc) is known in the art and has been previously described, e.g., by Semnza, Annu. Rev. Cell Dev. Biol. 1999, 51:551-578. At least one occurrence of this consensus sequence can in the CAP43 cDNA sequence depicted in FIG. 1A (SEQ ID NO: 1) at nucleic acid residue position 952. Moreover, the nucleic acid sequence immediately 5′- of the CAP43 gene has also been sequenced and is presented here in FIG. 10 (SEQ ID NO: 3). This nucleic acid sequence, which includes the CAP43 promoter region and ends at the ATG codon were the coding sequence for CAP43 (shown in FIG. 1A) begins, contains at least two HIF-1 binding sites at nucleic acid residue positions 743 and 1905 (indicated by bold-faced, underlined type in FIG. 10).

As another non-limiting example, a CAP43 nucleic acid or polypeptide may interact with one or more other compounds (for example, one or more other proteins or nucleic acids) to stabilize CAP43 in cells.

The screening assays of this invention may therefore be used to identify compounds that specifically bind to a CAP43 gene or gene product to modulate CAP43 expression. For example, the screening assays described here may therefore be used to identify compounds that bind to a promoter or other regulatory sequence of a CAP43 gene, and so may modulate the level of CAP43 gene expression (see, for example, Platt, J. Biol. Chem. 1994, 269:28558-28562). The screening assays may also be used to identify compounds that bind to and thereby stabilize a CAP43 nucleic acid or polypeptide. In addition, these screening assays may be used to identify compounds that inhibit or modulate such binding interactions and which are therefore useful, e.g., as agonists or antagonists for CAP43 binding to a specific transcription factor or enhancer, or for CAP43 binding to a stabilizer. Compounds identified in these or similar screening assays may therefore be used to treat diseases and disorders (for example, cancer) that are associated with abnormal CAP43 expression and/or abnormal levels of CAP43 polypeptides or nucleic acids.

Classes of compounds that may be identified by such screening assays include, but are not limited to, small molecules (e.g., organic or inorganic molecules which are less than about 2 kDa in molecular weight, are more preferably less than about 1 kDa in molecular weight, and/or are able to cross the blood-brain barrier or gain entry into an appropriate cell and affect expression of either a CAP43 gene or of some gene involved in a CAP43 regulatory pathway) as well as macromolecules (e.g., molecules greater than about 2 kDa in molecular weight). Compounds identified by these screening assays may also include peptides and polypeptides. Examples of such compounds (including peptides) include but are not limited to: soluble peptides; fusion peptide members of combinatorial libraries (such as ones described by Lam et al., Nature 1991, 354:82-84; and by Houghten et al., Nature 1991, 354:84-86); members of libraries derived by combinatorial chemistry, such as molecular libraries of D- and/or L-configuration amino acids; phosphopeptides, such as members of random or partially degenerate, directed phosphopeptide libraries (see, e.g., Songyang et al., Cell 1993, 72:767-778); antibodies, including but not limited to polyclonal, monoclonal, humanized, anti-idiotypic, chimeric or single chain antibodies; antibody fragments, including but not limited to Fab, F(ab′)₂, Fab expression library fragments, and epitope-binding fragments thereof.

Assays for Binding Compounds.

In vitro systems can be readily designed to identify compounds capable of binding the CAP43 gene products of the present invention. Such compounds can be useful, for example, in modulating the expression, stability or activity of a wild-type CAP43 gene product or, alternatively, to modulate the expression, stability or activity of a mutant or other variant CAP43 gene product.

Generally, such screening assays involve preparation of a reactive mixture comprising a CAP43 gene product and a test compound under conditions and for a time sufficient to allow the two compounds to interact (e.g., bind), thereby forming a complex that may be detected. The assays may be conducted in any of a variety of different ways. For example, one embodiment comprises anchoring a CAP43 polypeptide or a test compound onto a solid phase and detecting complexes of the CAP43 polypeptide and the test compound that are on the solid phase at the end of the reaction and after removing (e.g., by washing) unbound compounds. For example, in one preferred embodiment of such a method, a CAP43 gene product may be anchored onto a solid surface and a labeled compound (e.g., labeled according to any of the methods described supra) is contacted to the surface. After incubating the test compound for a sufficient time and under sufficient conditions that a complex may form between the CAP43 gene product and the test compound, unbound molecules of the test compound are removed from the surface (e.g., by washing) and labeled molecules which remain are detected.

In another, alternative embodiment, molecules of one or more different test compounds are attached to the solid phase and molecules of a labeled CAP43 polypeptide may be contacted thereto. In such embodiments, the molecules of different test compounds are preferably attached to the solid phase at a particular location on the solid phase so that test compounds that bind to a CAP43 polypeptide may be identified by determining the location of bound CAP43 polypeptides on the solid phase or surface.

Assays for Compounds That Interact With CAP43.

Any of a variety of known methods for detecting protein-protein interactions may also be used to detect and/or identify proteins that interact with a CAP43 gene product. For example, co-immunoprecipitation, cross-linking and co-purification through gradients or chromatographic columns as well as other techniques known in the art may be employed. Proteins which may be identified using such assays include, but are not limited to, extracellular proteins, such as CAP43 specific receptors and ligands, as well as intracellular proteins such as signal transducing proteins.

As an example, and not by way of limitation, an expression cloning assay may be used to identify CAP43 specific receptors and other proteins that specifically interact with a CAP43 gene product. In such assays, a cDNA expression library may be generated from any cell line that expresses a CAP43 specific receptor (for example, leukocyte cells, such as monocytes, B lymphocytes and T lymphocytes, including CD8 and CD4 cells). Clones from such an expression library may then be transfected or infected into cells, such as a B cell lymphoma line (e.g., CH12 cells, A20.25 cells or LBB1 cells) that do not normally express a CAP43 specific receptor. Cells that are transfected with a clone that encodes a CAP43 specific receptor may then express this gene product, and can be identified and isolated using standard techniques such as FACS or using magnetic beads that have a CAP43 polypeptide (for example, a CAP43-Fc fusion polypeptide) attached thereto.

Alternatively, a CAP43 specific receptor or ligand may be isolated from a cell line, including any of the CAP43 receptor expressing cell lines recited above, using immunoprecipitation techniques that are well known in the art.

CAP43 specific receptors and/or ligands may also be isolated using any of the screening assays discussed, supra for identifying CAP43 binding compounds. For example, a CAP43-Fc fusion polypeptide may be bound or otherwise attached to a solid surface, and a labeled compound (e.g., a candidate CAP43 receptor or ligand) may be contacted to the surface for a sufficient time and under conditions that permit formation of a complex between the CAP43-Fc fusion polypeptide and the test compound. Unbound molecules of the test compound can then be removed from the surface (e.g., by washing), and labeled compounds that remain bound can be detected.

Once so isolated, standard techniques may be used to identify any protein detected in such assays. For example, at least a portion of the amino acid sequence of a protein that interacts with the CAP43 gene product can be ascertained using techniques well known in the art, such as the Edman degradation technique (see, e.g., Creighton, 1983, Proteins: Structures and Molecular Principles, W. H. Freeman&Co., New York, pages 34-49).

Once such proteins have been identified, their amino acid sequence may be used as a guide for the generation of oligonucleotide mixtures to screen for gene sequences encoding such proteins; e.g., using standard hybridization or PCR techniques described supra. See, for example, Ausubel supra; and PCR Protocols: A Guide to Methods and Applications, Innis et al., eds., Academic Press, Inc., New York (1990) for descriptions of techniques for the generation of such oligonucleotide mixtures and their use in screening assays.

Other methods are known in the art which result in the simultaneous identification of genes that encode a protein that interacts with a CAP43 polypeptide. For example, expression libraries may be probed with a labeled CAP43 polypeptide.

As another example and not by way of limitation, a two-hybrid system may be used to detect protein interactions with a CAP43 gene product in vivo. Briefly, utilizing such a system, plasmids may be constructed which encode two hybrid proteins: one of which preferably comprises of the DNA-binding domain of a transcription activator protein fused to a CAP43 gene product. The other hybrid protein preferably comprises an activation domain of the transcription activator protein used in the first hybrid, fused to an unknown protein that is encoded by a cDNA recombined into the plasmid library as part of a cDNA library. Both the DNA-binding domain fusion plasmid and the cDNA library may be co-transformed into a strain of Saccharomyces cerevisiae or other suitable organism which contains a reporter gene (for example, HBS, lacZ, HIS3 or GFP). Preferably, the regulatory region of this reporter gene comprises a binding site for the transcription activator moiety of the two hybrid proteins. In such a two-hybrid system, the presence of either of the two hybrid proteins alone cannot activate transcription of the reporter gene. Specifically, the DNA-binding domain hybrid protein cannot activate transcription because it cannot localize to the necessary activation function. Likewise, the activation domain hybrid protein cannot activate transcription because it cannot localize to the DNA binding site on the reporter gene. However, interaction between the two hybrid proteins, reconstitutes that functional transcription activator protein and results in expression of the reporter gene. Thus, in a two-hybrid system such as the one described here in detail, an interaction between a CAP43 polypeptide (i.e., the CAP43 polypeptide fused to the transcription activator's DNA binding domain) and a test polypeptide (i.e., a protein fused to the transcription activator's DNA binding domain) may be detected by simply detecting expression of a gene product of the reporter gene.

cDNA libraries for screening in such two-hybrid and other assays may be made according to any suitable technique known in the art. As a particular and non-limiting example, cDNA fragments may be inserted into a vector so that they are translationally fused to the transcriptional activation domain of GAL4, and co-transformed along with a “bait” CAP43-GAL4 fusion plasmid into a strain of Saccharomyces cerevisiae or other suitable organism that contains a HIS3 gene driven by a promoter that contains a GAL4 activation sequence. A protein from this cDNA library, fused to the GAL4 transcriptional activation domain, which interacts with the CAP43 polypeptide moiety of the CAP43-GAL4 fusion will reconstitute and active GAL4 protein, and can thereby drive expression of the HIS3 gene. Colonies that express the HIS3 gene may be detected by their growth on petri dishes containing semi-solid agar based media lacking histidine. The cDNA may then be purified from these strains, sequenced and used to identify the encoded protein which interacts with the CAP43 polypeptide.

Once compounds have been identified which bind to a CAP43 gene product of the invention, the screening methods described in these methods may also be used to identify other compounds (e.g., small molecules, peptides and proteins) which bind to these binding compounds. Such compounds may also be useful to modulating CAP43 -related bioactivities, for example by binding to a natural CAP43 receptor, ligand or other binding partner, and presenting its interaction with a CAP43 gene product. For instance, these compounds could be tested for their ability to inhibit the binding of CAP43-Fc to cell lines which express CAP43 specific receptor.

Assays for Compounds That Interfere With A CAP43 Ligand Interaction.

As noted supra, a CAP43 gene product of the invention may interact with one or more molecules (i.e., receptors) in vivo or in vitro. Compounds that disrupt or otherwise interfere with this binding interaction are therefore useful in modulating the expression, stability or activity of a CAP43 gene product, and, in particular, may serve to enhance or suppress levels of CAP43 in cells and thereby to treat a disease or disorder (for example, cancer) that is associated with abnormal CAP43 expression.

Such compounds include, but are not limit to, compounds identified according to the screening assays described supra, for identifying compounds that bind to a CAP43 gene product, including any of the numerous exemplary classes of compounds described therein.

In general, assays for identifying compounds that interfere with the interaction between a CAP43 gene product and a binding partner (e.g., a receptor or ligand) involve preparing a test reaction mixture that contains the CAP43 gene product and its binding partner under conditions and for a time sufficient for the CAP43 gene product and its binding partner to bind and form a complex. In order to test a compound for inhibitory activity (i.e., for the ability to inhibit formation of the binding complex or to disrupt the binding complex once formed), the test compound preferably is also present in the test reaction mixture. In one exemplary embodiment, the test compound may be initially included in the test reaction mixture with the CAP43 gene product and its binding partner. Alternatively, however, the test compound may be added to the test reaction mixture at a later time, subsequent to the addition of the CAP43 gene product and its binding partner. In preferred embodiments, one or more control reaction mixtures, which do not contain the test compound, may also be prepared. Typically, a control reaction mixture will contain the same CAP43 gene product and binding partner that are in the test reaction mixture, but will not contain a test compound. A control reaction mixture may also contain a placebo, not present in the test reaction mixture, in place of the test compound. The formation of a complex between the CAP43 gene product and the binding partner may then be detected in the reaction mixture. The formation of such a complex in the absence of the test compound (e.g., in a control reaction mixture) but not in the presence of the test compound, indicates that the test compound is one which interferes with or modulates the interaction of a CAP43 polypeptide and a binding partner.

Such assays for compounds that modulate the interaction of a CAP43 gene product and a binding partner may be conducted in a heterogenous format or, alternatively, in a homogeneous format. Heterogeneous assays typically involve anchoring either a CAP43 gene product or a binding partner onto a solid phase and detecting compounds anchored to the solid phase at the end of the reaction. Thus, such assays are similar to the solid phase assays described supra for detecting and/or identifying CAP43 nucleic acids and gene products and for detecting or identifying CAP43 binding partners. Indeed, those skilled in the art will recognize that many of the principles and techniques described above for those assays may be modified and applied without undue experimentation in the solid phase assays described here, for identifying compounds that modulate interaction(s) between and CAP43 gene product and a binding partner.

Regardless of the particular assay used, the order to which reactants are added to a reaction mixture may be varied; for example, to identify compounds that interfere with the interaction of a CAP43 gene product with a binding partner by competition, or to identify compounds that disrupt a preformed binding complex. Compounds that interfere with the interaction of a CAP43 gene product with a binding partner by competition may be identified by conducting the reaction in the presence of a test compound. Specifically, in such assays a test compound may be added to the reaction mixture prior to or simultaneously with the CAP43 gene product and the binding partner. Test compounds that disrupt preformed complexes of a CAP43 gene product and a binding partner may be tested by adding the test compound to a reaction mixture after complexes have been formed.

The screening assays described herein may also be practiced using peptides or polypeptides that correspond to portions of a full length CAP43 polypeptide or protein, or with fusion proteins comprising such peptide or polypeptide sequences. For example, screening assays for identifying compounds the modulate interactions of a CAP43 polypeptide with a binding partner may be practiced using peptides or polypeptides corresponding to particular regions or domains of a full length CAP43 polypeptide that bind to a binding partner (e.g., receptor “binding sites”).

A variety of methods are known in the art that may be used to identify specific binding sites of a CAP43 polypeptide. For example, binding sites may be identified by mutating a CAP43 gene and screening for disruptions of binding as described above. A gene encoding the binding partner may also be mutated in such assays to identify mutations that compensate for disruptions from the mutation to the CAP43 gene. Sequence analysis of these mutations can then reveal mutations that correspond to the binding region of the two proteins.

In an alternative embodiment, a protein (e.g., a CAP43 protein or a protein binding partner to a CAP43 protein) may be anchored to a solid surface or support using the methods described herienabove. Another labeled protein which binds to the protein anchored to the solid surface may be treated with a proteolytic enzyme, and its fragments may be allowed to interact with the protein attached to the solid surface, according to the methods of the binding assays described supra. After washing, short, labeled peptide fragments of the treated protein may remain associated with the anchored protein. These peptides can be isolated and the region of the full length protein from which they are derived may be identified by the amino acid sequence.

In still other embodiments, compounds that interfere with a CAP43-receptor interaction may also be identified by screening for compounds that modulate binding of a CAP43 polypeptide (for example, a CAP43-Fc fusion polypeptide) to cells that express a CAP43 specific receptor, such as leukocyte cells (including monocytes, B lymphocytes and T lymphocytes, including CD8 and CD4 cells).

5.6.3. Therapeutic Methods and Pharmaceutical Preparations

CAP43 nucleic acids and polypeptides, and CAP43 specific antibodies may also be used in therapeutic methods and compositions, e.g., to treat diseases and disorders associated with abnormal (preferably elevated) levels of CAP43 expression. In preferred embodiments such methods are used to treat cancer, such as any of the types of cancers recited supra. In particular, Applicants have discovered that CAP43 polypeptides and nucleic acids are expressed in cancer cells and tissue at elevated levels relative to other (e.g., benign and/or normal) cells and tissue. CAP43 is also expressed in other diseased cells or tissue that are associated with hypoxic conditions; for example, artherosclerosis and granuloma. Accordingly, in one preferred embodiment the therpeutic methods of the involve administering one or more compounds (for example, a compound identified in any of the screening assays described, supra, in Section 5.6.2) that modulate (e.g., inhibit) CAP43 expression or activity; for example, compounds that bind to a CAP43 nucleic acid or polypeptide of the invention, compounds that modulate expression of a CAP43 gene, and compounds that interfere with or modulate binding of a CAP43 nucleic acid or polypeptide with a binding compound.

In another preferred embodiment, the therapeutic methods of the invention may comprise one or more cell-targeted therapies which target compounds (for example, drugs, pro-drugs, toxins or cytotoxins) to cells expressing a CAP43 nucleic acid or polypeptide.

Cell Targeted Therapies.

In preferred embodiments of the present invention, CAP43 nucleic acids and polypeptides, and CAP43 specific antibodies may be used to specifically target compounds, such as toxins, drugs or other therapeutic compounds, to cells (for example, to cancer cells) that express a CAP43 nucleic acid or gene product. Preferably, such methods are used to specifically target compounds to cells that express a CAP43 nucleic acid or polypeptide at elevated levels relative to normal cells, for example to any of the different types of cancer cells recited supra.

For example, the invention provides methods for the specific destruction of cells (e.g., the destruction of cancer cells) in a host or ex vivo (e.g., in short term or long term cell cultures) by specifically targeting compounds such as toxin and cytotoxins to the cells. Methods for cell specific targeting of toxins and cytotoxins are known in the art. For example, preferred methods involve complexing a toxin or cytotoxin with a cell binding protein that can preferentially bind to the target cells, e.g., by binding to a particular nucleic acid or polypeptide expressed by the targeted cells.

Accordingly, in one embodiment the invention provides methods for specifically targeting compounds (e.g., toxins and cytotoxins) to cells such as cancer cells. Such methods involve contacting to the cell with the compound complexed to a protein that specifically binds to a CAP43 polypeptide or nucleic acid expressed by the cell. In one embodiment of such methods the protein may be, e.g., a protein ligand that specifically binds to a CAP43 polypeptide. However, in preferred embodiments the protein is an CAP43 specific antibody.

Any compound may be targeted and administered to a cell according to such methods. However, in preferred embodiments the compound is a toxin or a cytoxin that destroys or inhibits the targeted cell or cells. Thus, in preferred embodiments the compound is an anti-cancer drug. Exemplary toxins that may be used according to such methods include, but are not limited to, thymidine kinase, endonuclease, RNAse, α-toxin, ricin, abrin, Pseudomonas exotoxin A, diphtheria toxin, saporin, momordin, gelonin, pokeweed antiviral protein, α-sarcin and cholera toxin.

Alternatively, a cytotoxic prodrug may be administered according to the therapeutic methods of the invention. Cytotoxic prodrugs are compounds that are, themselves, non-toxic but that may be converted into a toxic compound by an enzyme (preferably an enzyme that is normally or naturally presented in the targeted cell). Exemplary compounds which may be administered as prodrugs in the present invention include, but are not limited to, derivatives (preferably glutamyl derivatives) of a benzoic acid mustard alkylating agents, derivatives (preferably phosphate derivatives) of etoposide or mitomycin C, and derivatives (preferably phenoxyacetamide derivatives) of doxorubicin.

A variety of methods are know in the art that may be used to complex a compound (e.g., a drug, a pro-drug, a toxin or a cytotoxin) to an antibody or other CAP43 binding polypeptide (see, for example, Boado, Advanced Drug Delivery Reviews 1995, 15:73-107; Ozawa et al., Int. J. Cancer 1989, 43:152-157; Uhr & Vitetta, U.S. Pat. No. 4,664,911; Rybak et al., J. Biol. Chem. 1991, 266:21202-21207; and Tosi et al., Eur. J. Cancer 1991, 27(Suppl. 3):S57). For example, a compound (e.g, a drug or toxin) may be covalently linked to a CAP43 binding polypeptide (e.g., a CAP43 specific antibody) with a nonspecific heterobifunctional cross-linking reagent. Alternatively, in embodiments where the targeted compound is a protein or polypeptide, the compound may be complexed to the antibody by using recombinant DNA technology to generate a chimeric gene encoding a fusion protein where the amino acid sequence of the targeted polypeptide is fused to the Fab portion of an antibody that specifically binds CAP43.

In another embodiment, bispecific antibodies may be used to target compounds (e.g., drugs or toxins) to cells. Bispecific antibodies are known in the art and their use for targeting compounds to cells has been described, e.g., by Glennie, International Patent Publication No. WO 98/11863. In preferred embodiments, a bifunctional antibody used in the methods of this invention comprises a first Fab′ arm that specifically binds to the compound targeted to the cell or cells, and a second Fab′ arm that specifically binds to a CAP43 nucleic acid or polypeptide expressed by the targeted cell.

Still other techniques are known in the art by which a compound may be complexed non-covalently with an antibody. One such technique exploits a specific interaction between Staphylococcal aureus protein A and immunoglobulins to generate antibody complexes with two specificies. According to this technique, protein A is complexes with antibodies of two different specifities: an antibody specific for the targeted compound (e.g., the targeted drug or toxin) and a CAP43 specific antibody (see, e.g., Laky et al., Immunology Letters 1986/1987, 14:127-132).

In yet another targeting system, single chain antibodies may be fused with streptavidin which has a strong and specific binding affinity for biotin. Biotinylated compounds, such as biotinylated drugs and toxins) may then be delivered into targeted cells using such constructs (see, e.g., Dubel et al., Journal of Immunological Methods 1995, 178:201-209). Alternatively, Sano & Cantor (Bio/Technology 1991, 9:1371-1380; see, also, U.S. Pat. No. 5,328,985 issued Jul. 12, 1994 to Sano et al.) have described a fusion protein consisting of streptavidin and one or two immunoglobulin G (IgG)-binding domains of protein A. Such a fusion protein can be used to form a complex having (i) a biotinylated compound (e.g., a drug or toxin) bound to the streptavidin domain and (ii) an antibody bound to the IgG-binding domain of such a fusion protein, and using such complexes to target and administer the compound to cells (see, Meruelo et al., International Patent Publication No. WO 97/19957).

Inhibitory Approaches.

In alternative embodiments, the present invention provides methods and compositions for treating a disease or disorder (for example, cancer) associated with abnormal CAP43 expression or activity by modulating (e.g., increasing or decreasing) the expression or activity of a CAP43 gene or its gene product. Such methods may simply comprise administering one or more compounds that modulate expression of a CAP43 gene, synthesis of a CAP43 gene product or CAP43 gene product activity so the immune response is modulated (e.g., enhanced or suppressed). Preferably, these one or more compounds are administered until one or more symptoms of the disorder are eliminated or at least ameliorated.

Among the compounds that may exhibit an ability to modulate the activity, expression or synthesis of a CAP43 nucleic acid are antisense, ribozyme and triple-helix molecules. Such molecules may be designed to reduce or inhibit wild-type CAP43 nucleic acids and polypeptides or, alternatively, may target mutant CAP43 nucleic acids or polypeptides.

Antisense RNA and DNA molecules act to directly block the translation of mRNA by hybridizing to target mRNA molecules and preventing protein translation. Antisense approaches involve the design of oligonucleotides that are complementary to a target gene mRNA. The antisense oligonucleotides will bind to the complementary target gene mRNA transcripts and prevent translation. Absolute complementarity, although preferred, is not required.

A sequence that is “complementary” to a portion of a nucleic acid refers to a sequence having sufficient complementarity to be able to hybridize with the nucleic acid and form a stable duplex. The ability of nucleic acids to hybridize will depend both on the degree of sequence complementarity and the length of the antisense nucleic acid. Generally, however, the longer the hybridizing nucleic acid, the more base mismatches it may contain and still form a stable duplex (or triplex in triple helix methods). A tolerable degree of mismatch can be readily ascertained, e.g., by using standard procedures to determine the melting temperature of a hybridized complex.

In one preferred embodiment, oligonucleotides complementary to non-coding regions of a CAP43 gene may be used in an antisense approach to inhibit translation of endogenous CAP43 mRNA molecules. Antisense nucleic acids are preferably at least six nucleotides in length, and more preferably range from between about six to about 50 nucleotides in length. In specific embodiments, the oligonucleotides may be at least 10, at least 15, at least 20, at least 25 or at least 50 nucleotides in length.

It is generally preferred that in vitro studies are first performed to quantitate the ability of an antisense oligonucleotide to inhibit gene expression. It is preferred that these studies utilize controls that distinguish between antisense gene inhibition and nonspecific biological effects of oligonucleotides. It is also preferred that these studies compare levels of the target RNA or protein with that of an internal control RNA or protein. Additionally, it is envisioned that results obtained using the antisense oligonucleotide are compared with those obtained using a control oligonucleotide. It is preferred that the control oligonucleotide is of approximately the same length as the test oligonucleotide and that the nucleotide sequence of the oligonucleotide differs from the antisense sequence no more than is necessary to prevent specific hybridization to the target sequence.

While antisense nucleotides complementary to the target gene coding region sequence could be used, those complementary to the transcribed, untranslated region are most preferred.

Antisense molecules are preferably delivered to cells, such as cancer cells, that express the target gene in vivo. A number of methods have been developed for delivering antisense DNA or RNA to cells. For example, antisense molecules can be injected directly into the tissue site (e.g., directly into a tumor), or modified antisense molecules can be designed to target the desired cells (e.g., antisense linked to peptides or antibodies that specifically bind receptors or antigens expressed on the target cell surface) can be administered systemically.

Preferred embodiments achieve intracellular concentrations of antisense nucleic acid molecules which are sufficient to suppress translation of endogenous mRNAs. For example, one preferred approach uses a recombinant DNA construct in which the antisense oligonucleotide is placed under the control of a strong pol III or pol II promoter. The use of such a construct to transfect target cells in the patient will result in the transcription of sufficient amounts of single stranded RNAs that will form complementary base pairs with the endogenous target gene transcripts and thereby prevent translation of the target gene mRNA. For example, a vector, as set forth above, can be introduced e.g., such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in the particular cell type (for example in a hemopoietic cell). For example, any of the promoters discussed supra in connection with the expression of recombinant CAP43 nucleic acids can also be used to express a CAP43 antisense nucleic acid.

Ribozyme molecules designed to catalytically cleave target gene mRNA transcripts can also be used to prevent translation of target gene mRNA and, therefore, expression of target gene product (see, e.g., International Publication No. WO 90/11364; Sarver, et al., Science 1990, 247:1222-1225).

Ribozymes are enzymatic RNA molecules capable of catalyzing the specific cleavage of RNA (for a review, see Rossi, Current Biology 1994, 4:469-471). The mechanism of ribozyme action involves sequence specific hybridization of the ribozyme molecule to complementary target RNA, followed by an endonucleolytic cleavage event. The composition of ribozyme molecules must include one or more sequences complementary to the target gene mRNA, and must include the well known catalytic sequence responsible for mRNA cleavage. For this sequence, see, e.g., U.S. Pat. No. 5,093,246.

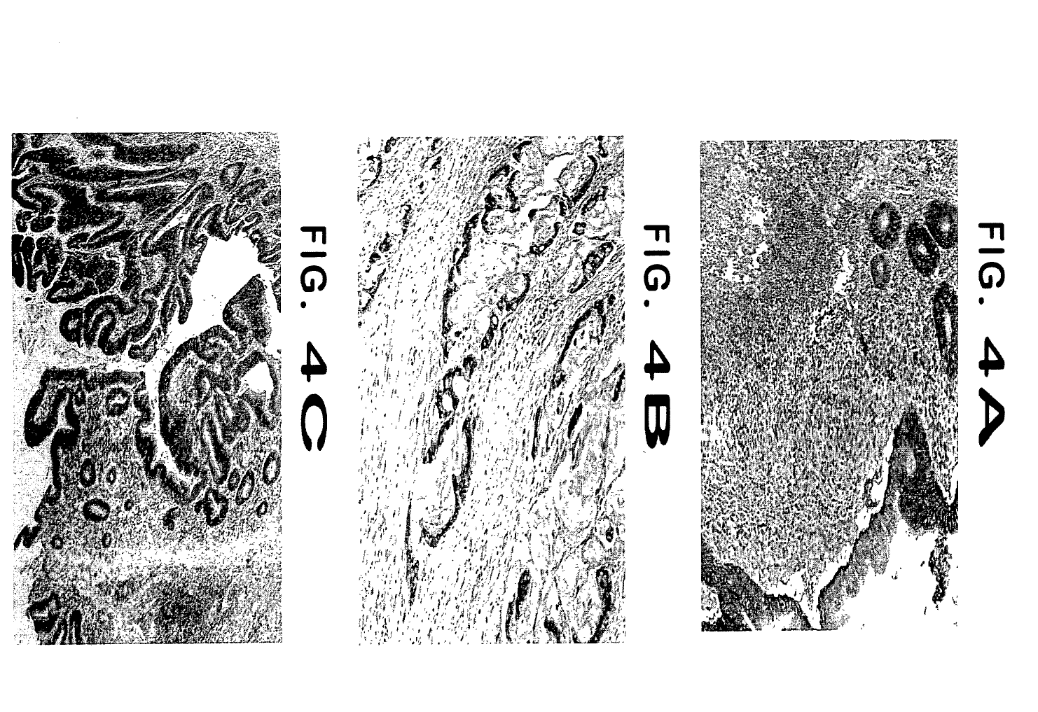

While ribozymes that cleave mRNA at site specific recognition sequences can be used to destroy target gene mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Myers, 1995, Molecular Biology and Biotechnology: A Comprehensive Desk Reference, VCH Publishers, New York, (see especially FIG. 4, page 833) and in Haseloff and Gerlach, Nature 1988, 334:585-591.

Preferably the ribozyme is engineered so that the cleavage recognition site is located near the 5′ end of the target gene mRNA, i.e., to increase efficiency and minimize the intracellular accumulation of non-functional mRNA transcripts.

The ribozymes of the present invention also include RNA endoribonucleases (hereinafter “Cech-type ribozymes”) such as the one that occurs naturally in Tetrahymena thermophila (known as the IVS, or L-19 IVS RNA) and that has been extensively described by Thomas Cech and collaborators (Zaug, et al., Science 1984, 224:574-578; Zaug and Cech, Science 1986, 231:470-475; Zaug et al., Nature 1986, 324:429-433; International Patent Publication No. WO 88/04300; Been and Cech, Cell 1986, 47:207-216). The Cech-type ribozymes have an eight base pair active site which hybridizes to a target RNA sequence whereafter cleavage of the target RNA takes place. The invention encompasses those Cech-type ribozymes which target eight base-pair active site sequences that are present in the target gene.

As in the antisense approach, the ribozymes can be composed of modified oligonucleotides (e.g., for improved stability, targeting, etc.) and should be delivered to cells that express the target gene in vivo. A preferred method of delivery involves using a DNA construct “encoding” the ribozyme under the control of a strong constitutive pol III or pol II promoter, so that transfected cells will produce sufficient quantities of the ribozyme to destroy endogenous target gene messages and inhibit translation. Because ribozymes (unlike antisense molecules) are catalytic, a lower intracellular concentration is required for efficacy. Such constructs can be introduced to cells using any of the vectors described supra.

Endogenous target gene expression can also be reduced by inactivating or “knocking out” the target gene or its promoter using targeted homologous recombination (e.g., see Smithies, et al., Nature 1985, 317:230-234; Thomas and Capecchi, Cell 1987, 51:503-512; and Thompson et al., Cell 1989, 5:313-321). For example, a mutant, non-functional target gene (or a completely unrelated DNA sequence) flanked by DNA homologous to the endogenous target gene (either the coding regions or regulatory regions of the target gene) can be used, with or without a selectable marker and/or a negative selectable marker, to transfect cells that express the target gene in vivo. Insertion of the DNA construct, via targeted homologous recombination, results in inactivation of the target gene. Such approaches are particularly suited in the agricultural field where modifications to ES (embryonic stem) cells can be used to generate animal offspring with an inactive target gene (e.g., see Thomas and Capecchi, 1987 and Thompson, 1989, supra). However this approach can be adapted for use in other species of organism (for example, in fish, such as trout and salmon, and in mammals, including humans) provided the recombinant DNA constructs are directly administered or targeted to the required site in vivo using appropriate viral vectors.

Alternatively, endogenous target gene expression can be reduced by targeting deoxyribonucleotide sequences complementary to the regulatory region of the target gene (i.e., the target gene promoter and/or enhancers) to form triple helical structures that prevent transcription of the target gene in target cells in the body. (see generally, Helene, Anticancer Drug Des. 1991, 6:569-584; Helene, et al., Ann. N.Y. Acad. Sci. 1992, 660:27-36; and Maher, Bioassays 1992, 14:807-815).

Nucleic acid molecules to be used in triplex helix formation for the inhibition of transcription should be single stranded and composed of deoxynucleotides. The base composition of these oligonucleotides must be designed to promote triple helix formation via Hoogsteen base pairing rules, which generally require sizeable stretches of either purines or pyrimidines to be present on one strand of a duplex. Nucleotide sequences may be pyrimidine-based, which will result in TAT and CGC⁺ triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, contain a stretch of G residues. These molecules will form a triple helix with a DNA duplex that is rich in GC pairs, in which the majority of the purine residues are located on a single strand of the targeted duplex, resulting in GGC triplets across the three strands in the triplex.

Alternatively, the potential sequences that can be targeted for triple helix formation may be increased by creating a so called “switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or pyrimidines to be present on one strand of a duplex.

Pharmaceutical Preparations.

Compositions used in the therapeutic methods of this invention may be administered (e.g., in vitro or ex vivo to cell cultures, or in vivo to an organism) at therapeutically effective doses to treat a disease or disorder such as cancer. For example, complexes of a drug or toxin (including pro-drugs and cytotoxins) and a protein or antibody that specifically binds to CAP43 nucleic acid or polypeptide may be administered so that the drug or toxin is targeted and delivered to cells (for example, cancer cells) that express a CAP43 nucleic acid or polypeptide. Accordingly, the invention also provides pharmaceutical preparations for use, e.g., as therapeutic compounds for the treatment of disorders such as cancer (including any of the different types of cancer recited herein).

The terms “therapeutically effective dose” and “effective amount” refer to the amount of the compound that is sufficient to result in a therapeutic response. In embodiments where a compound (e.g., a drug or toxin) is administered in a complex (e.g., with a CAP43 specific antibody), the terms “therapeutically effective dose” and “effective amount” may refer to the amount of the complex that is sufficient to result in a therapeutic response. A therapeutic response may be any response that a user (e.g., a clinician) will recognize as an effective response to the therapy. Thus, a therapeutic response will generally be an amelioration of one or more symptoms of a disease or disorder. In preferred embodiments, where the pharmaceutical preparations are used to treat a cancer, a therapeutic response may be a reduction in the number of cancer cells observed, e.g., in biopsies from a patient during treatment. Alternatively, an effective therapeutic response may be a reduction or shrinkage in the size of one or more tumors.

Toxicity and therapeutic efficacy of compounds can be determined by standard pharmaceutical procedures, for example in cell culture assays or using experimental animals to determine the LD₅₀ and the ED₅₀. The parameters LD₅₀ and ED₅₀ are well known in the art, and refer to the doses of a compound that are lethal to 50% of a population and therapeutically effective in 50% of a population, respectively. The dose ratio between toxic and therapeutic effects is referred to as the therapeutic index and may be expressed as the ratio: LD₅₀/ED₅₀. Compounds that exhibit large therapeutic indices are preferred. While compounds that exhibit toxic side effects may be used. However, in such instances it is particularly preferable to use delivery systems that specifically target such compounds to the site of affected tissue so as to minimize potential damage to other cells, tissues or organs and to reduce side effects.

Data obtained from cell culture assay or animal studies may be used to formulate a range of dosages for use in humans. The dosage of compounds used in therapeutic methods of the present invention preferably lie within a range of circulating concentrations that includes the ED₅₀ concentration but with little or no toxicity (e.g., below the LD₅₀ concentration). The AS particular dosage used in any application may vary within this range, depending upon factors such as the particular dosage form employed, the route of administration utilized, the conditions of the individual (e.g., patient), and so forth.

A therapeutically effective dose may be initially estimated from cell culture assays and formulated in animal models to achieve a circulating concentration range that includes the IC₅₀. The IC₅₀ concentration of a compound is the concentration that achieves a half-maximal inhibition of symptoms (e.g., as determined from the cell culture assays). Appropriate dosages for use in a particular individual, for example in human patients, may then be more accurately determined using such information.

Measures of compounds in plasma may be routinely measured in an individual such as a patient by techniques such as high performance liquid chromatography (HPLC) or gas chromatography.

Pharmaceutical compositions for use in accordance with the present invention may be formulated in conventional manner using one or more physiologically acceptable carriers or excipients.

Thus, the compounds and their physiologically acceptable salts and solvates may be formulated for administration by inhalation or insufflation (either through the mouth or the nose) or oral, buccal, parenteral or rectal administration.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound. For buccal administration the compositions may take the form of tablets or lozenges formulated in conventional manner. For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration.

### 6. EXAMPLES

The present invention is also described by means of examples, including the particular Examples presented here below. The use of such examples anywhere in the specification is illustrative only and in no way limits the scope and meaning of the invention or of any exemplified term. Likewise, the invention is not limited to any particular preferred embodiments described herein. Indeed, many modifications and variations of the invention will be apparent to those skilled in the art upon reading this specification and can be made without departing from its spirit and scope. The invention is therefore to be limited only by the terms of the appended claims along with the full scope of equivalents to which the claims are entitled.

### 6.1. Example 1

### CAP43 Staining of Cancerous and Benign Tissue Samples

This example presents data from immunohistochemistry experiments in which a variety of different tissue samples were stained with a biotin-labeled polyclonal antibody, prepared according to routine methods described in Section 5.5 supra, that specifically bound to a CAP43 polypeptide having the amino acid sequence shown in FIG. 1B (SEQ ID NO: 2). The results of these experiments demonstrate that CAP43 is expressed at elevated levels in a variety of different types of cancer tissues, as well as other tissues and cell types (e.g., granuloma) indicative of a disease or disorder. The data show that CAP43 is an effective marker and may be used to identify such diseased tissue and to distinguish the diseased tissue from healthy or benign tissues.

The immunohistochemistry experiments were performed on formalin fixed parafin embedded tissues with appropriate positive and negative controls using an NexES automated immunohistochemical stainer (Ventana Medical Systems, Tucson, Ariz.). Tissue blocks were selected from archival residual pathologic materials from the surgical pathology files of the Kaplan Comprehensive Cancer Center at Bellevue Hospital (New York University School of Medicine, New York, N.Y.). Using an eight millimeter dermatological punch biopsy instrument, eight millimeter punch biopsies were created from the tissue blocks. These punches were re-embeded in paraffin to create a multitissue block for analysis. Five micron thick tissue sections were prepared onto charged glass slides and baked in an oven at 40° C. overnight.

The tissue sections were deparaffinized through three washes of 100% xylene followed by three washes through graded alcohols (100%, 90%) back to an aqueous state. The tissue sections were subjected to antigen retrieval by microwaving the tissue sections in a 0.1 M citrate buffer, pH=6, for 15 minutes. The tissue sections were allowed to cool and were placed into the NexES instrument. A biotinylated CAP43 -specific rabbit polyclonal antibody was used at a dilution of 1:200 and incubated at room temperature for two hours. Detection of bound antibody was accomplished using a standard streptavin-biotin horseradish peroxidase complex with 3,3-diaminobenzidine (DAB) as the chromagen. Positive staining of tissue was considered to be cytoplasmic or membranous.

Table I, below, indicates the different types of human tissue that were stained and anlyzed for CAP43 expression according to these methods. The degree or extent of CAP43-specific staining is indicated qualitatively in the Table as either no staining (−), low and/or differential staining (+), high staining (++) or very high staining (+++). Micrographs of each stained tissue type are also provided, in FIGS. 2A-8D as indicated in Table I.

These results show that CAP43 is not expressed in most normal, healthy tissue or is only differentially expressed at low levels. Interestingly, even brain tissue stains negatively for CAP43. This is an unexpected result as more genes are expressed in brain tissue than in any other tissue or organ of the human body.

CAP43 is not only useful as a marker for cancerous cells, but may also be used to identify other cell types as well. For example, FIG. 7C shows a micrograph of a granuloma tissue section that has been stained with a biotinylated CAP43-specific antibody. This tissue stains similarly to the other cancerous tissues, indicating that CAP43 is also a marker for other tissues types, such as granuloma.

Although the level of CAP43-specific staining increases in most cancerous tissues relative to benign tissue, CAP43 expression actually appears to decrease in malignant colon tissue compared to benign colon tissue samples (e.g., compare FIGS. 4B-C to FIG. 4A). Thus, in the particular case of colon cancer, cancerous cells and tissues may actually be identified and/or distinguished from benign cells and tissue by reduced levels of CAP43 expression.

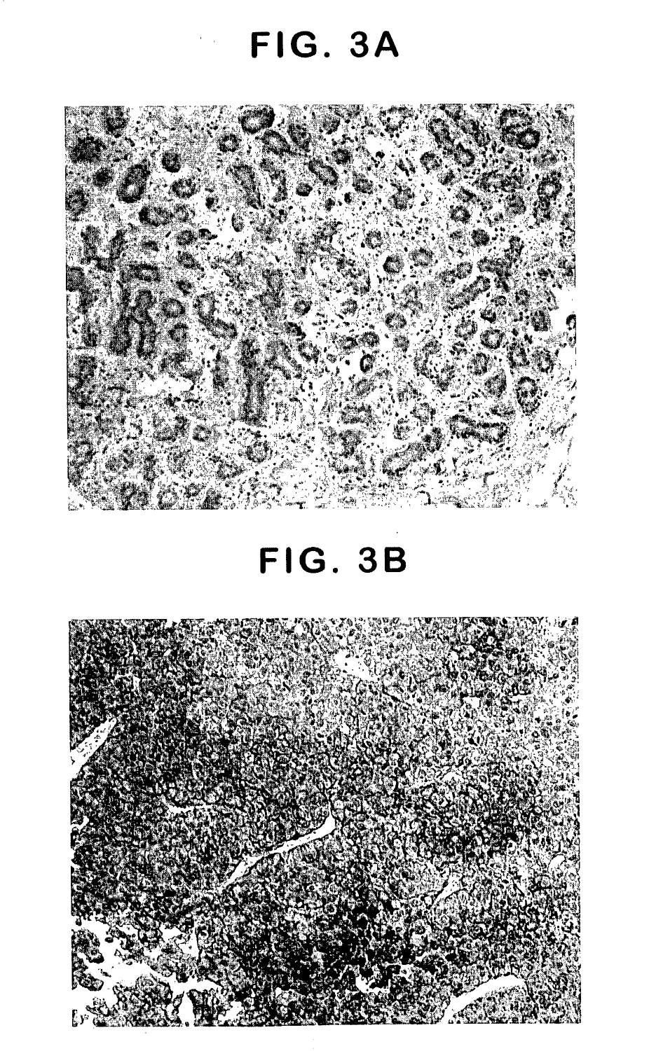

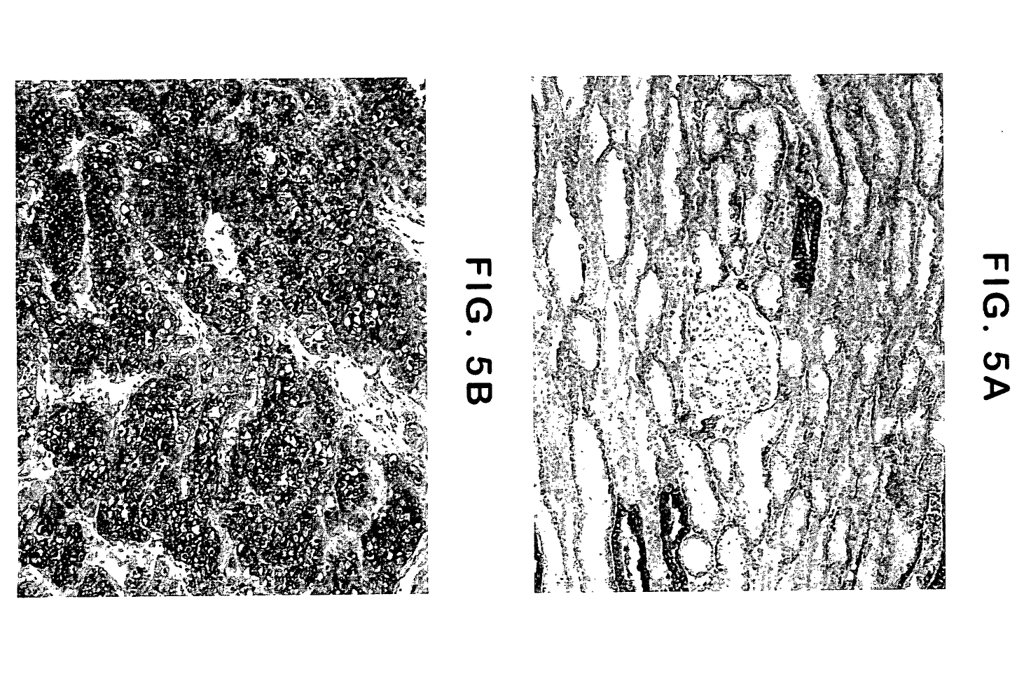

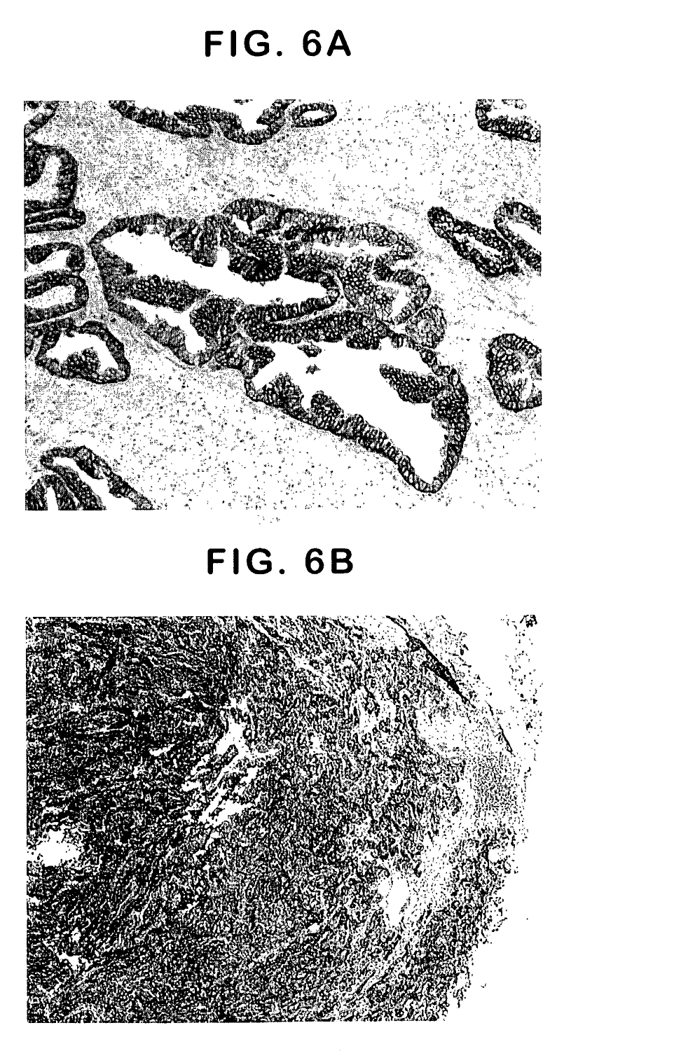

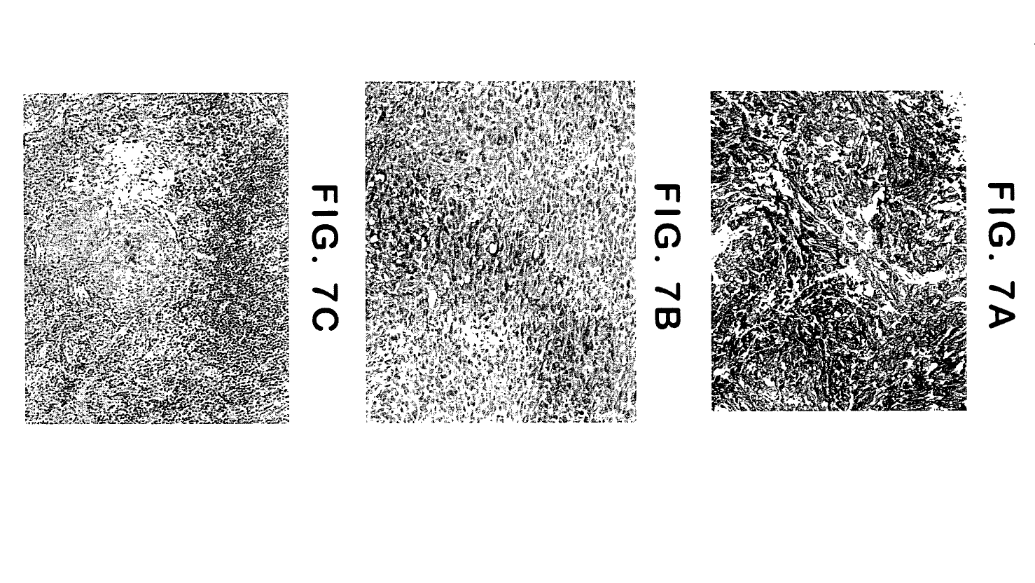

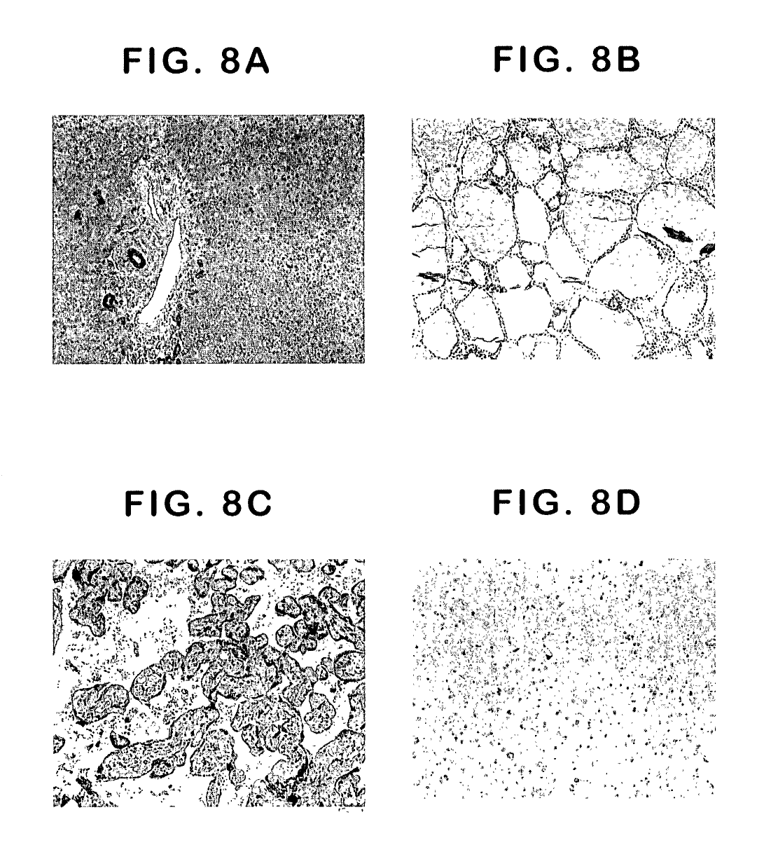

TABLE I
CAP43-SPECIFIC STAINING
IN HUMAN TISSUE SECTIONS

| tissue | staining | comment(s) |
|---|---|---|
| lung (benign) | (−) | See, FIG. 2A |
| lung (malignant) | (++) | See, FIG. 2B |
| breast (benign) | (+) | Low intensity staining only at |
| | | the ducts. See, FIG. 3A |
| breast (malignant) | (++) | See, FIG. 3B |
| colon (benign) | (++) | See, FIG. 4A |
| colon (malignant, | (+) | See, FIG. 4B |
| mucinous type) | | |
| colon (malignant, | (+) | See, FIG. 4C |
| tubular adenoma) | | |
| kidney (benign) | (+) | Differential staining of tubules, |
| | | with the distal convoluted tubule |
| | | staining darkest. |
| | | The glomerulus (center) of the |
| | | kidney tissue is completely |
| | | negative. See, FIG. 5A |
| kidney (malignant) | (++) | Staining throughout the tissue, |
| prostate (benign) | (+) | Differential staining. |
| | | See, FIG. 6A |
| prostate (malignant) | (++) | Uniform staining. See, FIG. 6B |
| melanoma | (+++) | See, FIG. 7A |
| malignant fibrous | (++) | See, FIG. 7B |
| histiocytoma | | |
| granuloma | (++) | See, FIG. 7C |
| liver (benign) | (+) | Only the bile duct in the portal |
| | | tract stains. See, FIG. 8A |
| thyroid (benign) | (−) | See, FIG. 8B |
| placenta (normal/benign) | (−) | See, FIG. 8C |
| brain (normal/benign) | (−) | See, FIG. 8D |

### 6.2. Example 2

### EXPRESSION OF CAP43 NUCLEIC ACIDS IN CANCER CELLS

This example presents data from Northern experiments performed using various types of cancer cells that were cultured in vitro, and demonstrates that CAP43 nucleic acids are expressed at elevated levels in these cells. In particular, CAP43 mRNA levels were measured for each of the cancer cell lines identified in TABLE II below. Each of these cell lines was ordered from the American Type Culture Collection (ATCC), Manassas, Virginia or has been previously described and are publicly available as indicated. The cells were cultured under standard, non-hypoxic conditions and pursuant to the depository's recommendations.

TABLE II
CULTURED CELL LINES

| Cell Line | Accession No. | Tissue |
|---|---|---|
| SW620 | CCL-227 | Colon carcinoma |
| KM12 | —⁽¹⁾ | Colon carcinoma |
| HCT15 | CCL-225 | Colon carcinoma |
| HCC2998 | —⁽²⁾ | Colon carcinoma |
| HCT116 | CCL-247 | Colon carcinoma |
| A549 | CCL-185 | Lung adenocarcinoma |
| NCI-H460 | HTB-177 | Lung adenocarcinoma |
| M14 | —⁽³⁾ | Melanoma |

⁽¹⁾Morikawa et al., Cancer Res. 1998, 48:6863-6871.
⁽²⁾Goldwasser et al., Cancer Res. 1995, 15:2116-2121.
⁽³⁾Chee et al., Cancer Res. 1976, 36:1503-1509.

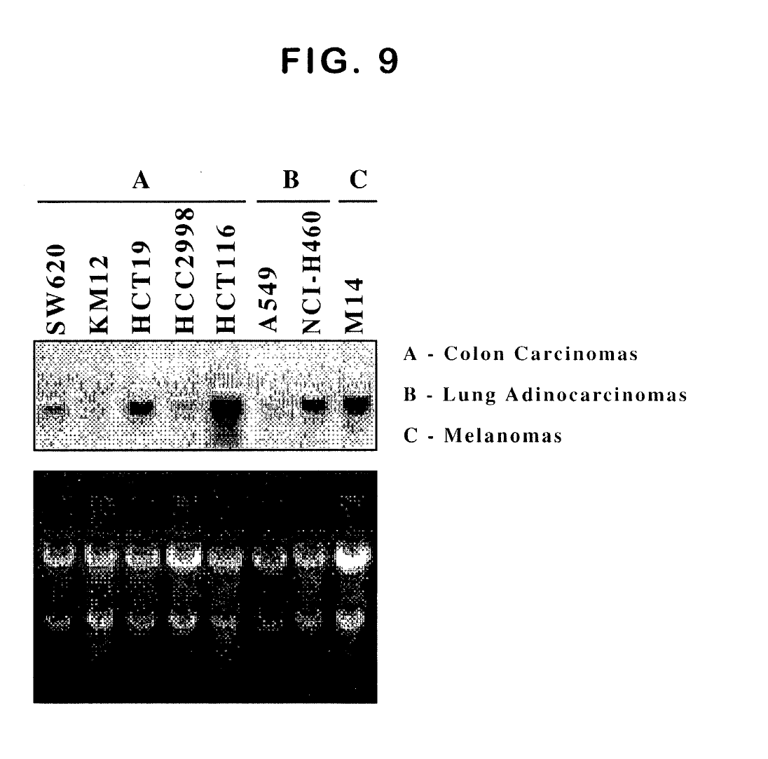

CAP43 mRNA levels in these cells were determined by standard Northern blot assays using a nucleic acid probe that comprised a nucleotide sequence complementary to the CAP43 coding sequence shown in FIG. 1A (i.e., complementary to nucleotides 29-1213 of SEQ ID NO: 1). Northern blots from each cell line are shown in FIG. 9 (top pannel). Total RNA extracts were also run on agarose gels a stained with ethedium bromide (FIG. 9; bottom panel) to confirm that each Northern indicated levels of CAP43 expression in similar levels of total cellular RNA. CAP43 nucleic acid is expressed at especially high levels in melanomas and in certain colon carcinoma cell lines (e.g., HCT19 and HCT116). Detectable levels of CAP43 nucleic acid were also observed in the lung adinocarcinoma cell line A549, consistent with what has been previously reported.

### 6.3. Example 3

### Enhanced Staining of CAP43 in Cancer Cells

This example presents additional data from immunohistochemistry experiments, and complements the immunohistochemistry data set forth in Example 1, supra. Specifically, these data confirm that the CAP43 protein is expressed in a variety of different types of cancer cells and tissue, while being expressed only at very low levels (if at all) in benign, healthy cells and tissue. Moreover, the data also demonstrate that CAP43 is superior to other cancer-specific genes reported in the art, such as CYP 1 B 1.

Materials and Methods.

Immunostaining experiments were performed as described, supra, in Section 6.1. Tumor and normal tissue sections were obtained from the tumor registry of the Kaplan Comprehensive Cancer Center (New York University School of Medicine, New York, N.Y.). Tumors and normal tissue were processed and imbedded in paraffin wax following standard protocols. Five micron sections were cut, deparaffinised using xylol and stained with hematoxylin-easin (H&E) for histopathological diagnosis. For immunochemical detection, slides were heated in a microwave oven for 10 minutes and in a citrate buffer. Endogenous peroxide was blocked with methanol containing 0.35% H₂O₂ for 30 minutes.

A rabbit polyclonal antibody was made against a 30 amino acid segment toward the N-terminal portion of the CAP43 amino acid sequence set forth in FIG. 1B (SEQ ID NO: 2). These 30 amino acids consisted of three 10 amino acid repeats. Western blots (described infra) confirmed that this antibody specifically stains only protein having the same molecular weight as CAP43 (43 kDa).

The CAP43-specific antibody thus obtained was incubated (1: 1000 dilution) with the tissue sections, and antibody binding to the tissue was detected using routine Avidin-Biotin horseradish peroxidase complex and 3,3-diaminobenzidine (DAB) as the chromagen. Negative controls were performed with nonimmune serum instead of primary antibodies.

Normal and cancerous tissues were also stained with an antibody specific for the hypoxia-inducible transcription factor, HIF-1α and for the protein CYB1B1, a protein that has also been reported as a cancer-specific marker (see, Murray et al., Cancer Res. 1997, 57:3026-3031). These antibodies were obtained from the commercial providers Neo Markers (Union City, Calif.) and GENTEST Corporation (Woburn, Mass.), respectively.

For Wester blot analysis, cells were lysed in TNES buffer that contained 50 mM Tris HCl (pH 7.5), 2 mM EDTA, 100 mM NaCl, 1 mM sodium orthovanadate and 1% NP40 containing protease inhibitors (20 μg/ml leupeptin and 1 mM phenylmethylsulfonyl fluoride). HIF-1α and CAP43 were detected as previously described (see, Salnikow et al., Carcinogenesis 1999, 20:1819-1823).

Results and Discussion.

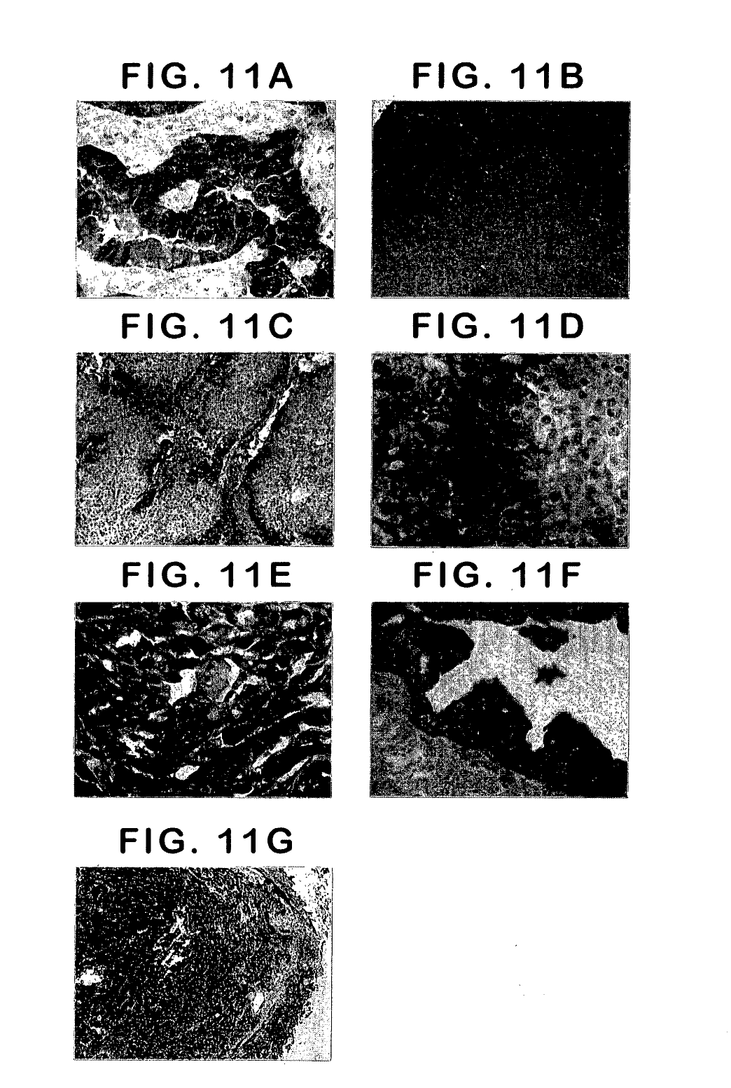

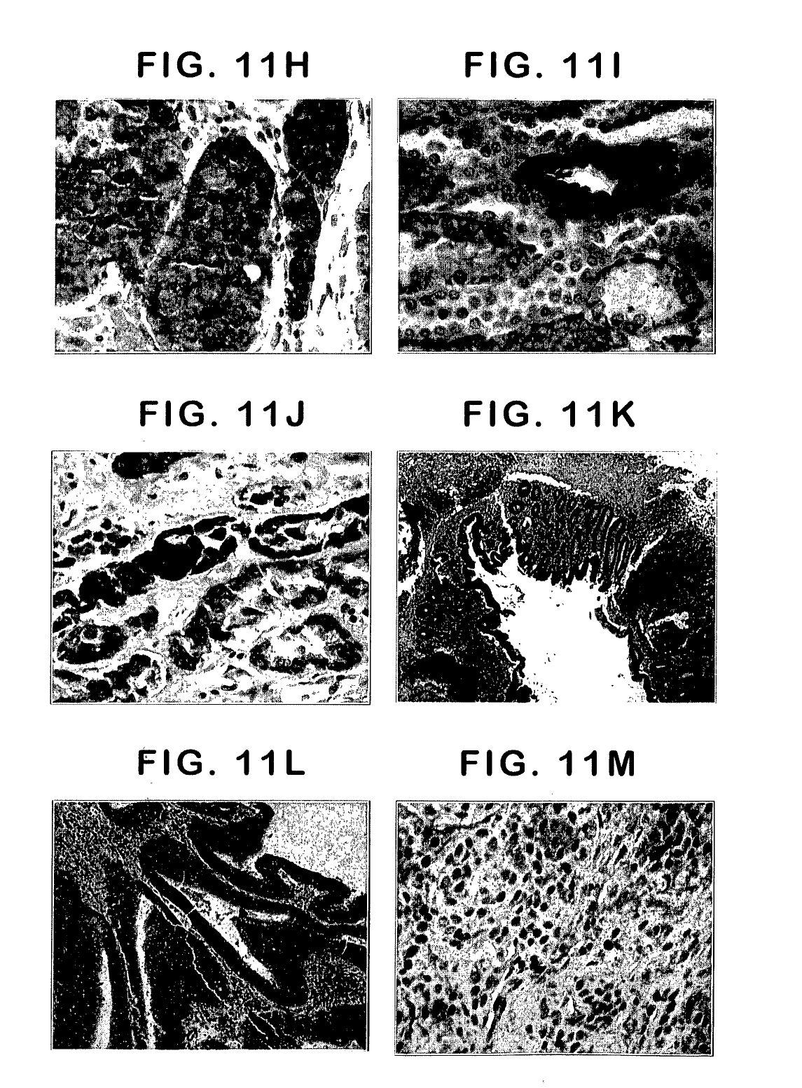

The experiments described here examined the levels of CAP43 protein in a variety of human normal and cancer cells, using immunohistochemical staining. An antibody was raised against a 30 amino acid sequence at the N-terminal end of the CAP43 amino acid sequence set forth in FIG. 1B and in SEQ ID NO: 2. FIGS. 11A-M show a variety of normal and cancer cells stained with this CAP43-specific polyclonal antibody. To understand whether elevations of CAP43 in these cells correlate with a state of hypoxia, tissues transcription factors, such as HIF-1α, are known to mediate transcriptional response to hypoxia (Forsythe et al., Mol. Cell Biol. 1996, 16:4606-4613; Semenza et al., J. Biol. Chem. 1996, 271:32529-32537) and are believed to be required for CAP43 induction by hypoxia (Salnikow et al., Cancer Res. 2000, 60:38-41).

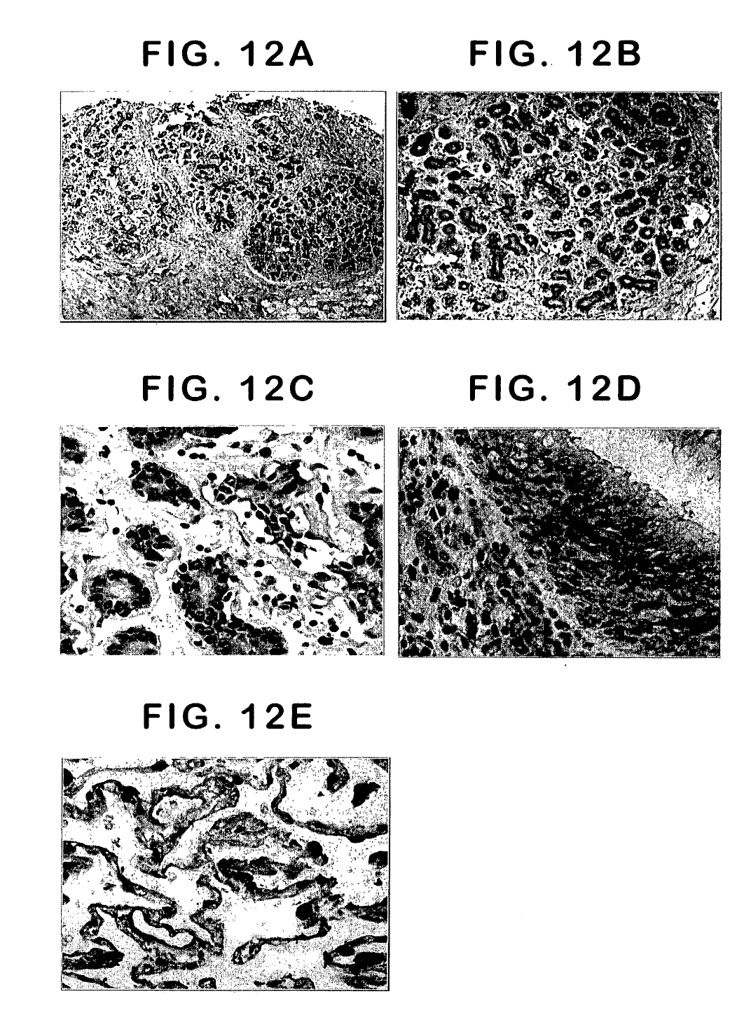

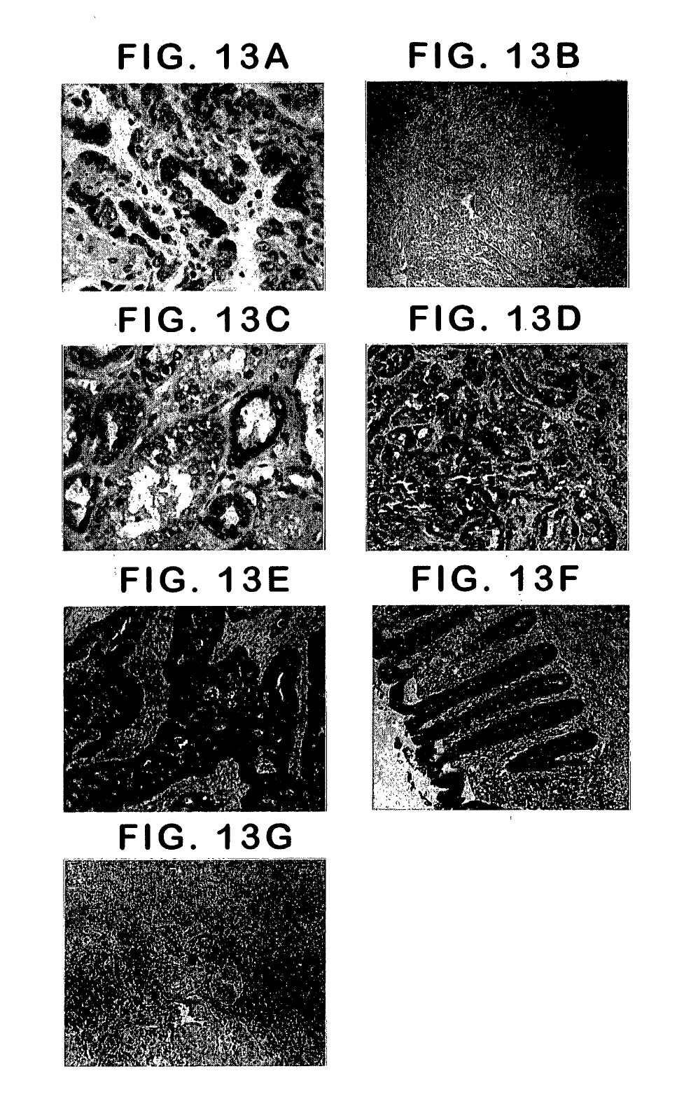

FIG. 11A shows a photomicrograph of lung cancer tumor cells, as well as surrounding normal tissue. FIGS. 12D and 12E show photomicrographs of normal lung tissue, including the broncheus an alveolar tissue. A visual inspection of these photomicrographs shows that these normal tissue have very low expression of CAP43 compared to the cancer tissues shown in FIG. 11. In contrast, HIF-1α is seen in both normal and lung cancer cells (FIG. 13A). Visual inspection of FIG. 13A does show that HIF-1α is present at higher levels in at least some cancer cells, but not to the extent observed for CAP43. CAP43 stains the nucleus as well as the cytoplasm of the neoplastic cells, but non-neoplastic lung cells (including cells of the broncheus and alveolar) stain very poorly with the CAP43 antibody if at all (see, FIGS. 12D and 12E).

CAP43 protein is generally found at only low levels in most healthy tissue, if at all. However, some higher expression of CAP43 is observed in the distal and proximal convoluted tubules of the kidney (FIG. 11I), and these tissues also express HIF-1α (FIG. 13C). CAP43 protein expression is also detected in colon mucosa, colon smooth muscle and normal prostate, as well as some expression in normal breast cells (FIGS. 12A-C) and in normal lung tissue (FIGS. 12D and 12E). However, the expression of CAP43 in cancer cells of those tissues is considerably higher and/or very different from what is observed in normal tissue (see, FIGS. 11 [??]).

FIG. 11B shows that there is oniy a low level of CAP43 protein expression in normal human brain. By contrast, FIG. 11C shows a low magnification view of a malignant human glioblastoma multiform and FIG. 11D shows a higher magnification of this brain tumor and the surrounding normal tissue. Visual inspection of these figures shows that CAP43-specific antibody preferentially stains the tumor cells adjacent to the necrotic areas. CAP43-specific antibody staining in these cells is localized to the membrane. Staining with the HIF-1α specific antibody similarly stains the same areas in these tissues (see, FIG. 13G), confirming that the induction of HIF-1α by hypoxia may be an important mechanism for the elevated expression of CAP43 seen in those malignant cells.

FIG. 11E shows a photomicrograph of a malignant melanoma with intense CAP43 staining in the cancer cells. However, the melanoma has little staining for HIF-1α (FIG. 13B). Normal skin (shown in FIG. 11M) shows little CAP43 staining by the CAP43-specific antibody.

FIG. 11F shows hyperplastic prostatic epithelial cells that stain positively for CAP43. FIG. 11G shows a low magnification of CAP43 staining of a high histological grade prostatic adenocarcinoma. The CAP43 staining is intense in these tumor cells, and is very different from the staining observed in normal cells.

FIG. 11H shows a photomicrograph from human breast in which an invasive ductal carcinoma stains intensely for CAP43, whereas normal breast cells (FIGS. 12A-C) stain poorly with the CAP43-specific antibody. In contrast to CAP43, HIF-1α specific staining is not as high in breast cancer cells. The distribution of CAP43 in these cancer cells is appears to be localized to the membrane predominantly, and focally to the cytoplasm.

FIG. 11I shows staining of the proximal and distal tubulues of normal kidney tissue. There is no staining for either HIF-1α or CAP43 in the glomerulus. However, differential staining of the tubular system is observed for both CAP43 (FIG. 11I) and for HIF-1α (FIG. 13C). The proximal tubes apparently have polarized membrane staining while the distal convoluted tubules have homogeneous cytoplasmic staining for CAP43 (see, FIG. 11I).

FIG. 11J shows a photomicrograph of renal cell carcinoma that demonstrates diffused cytoplasmic staining for CAP43. FIG. 11K shows benign colon tissue, and FIG. 11L shows an adenoma of the colon. Consistent with what has been previously reported (see, van Belzen et al., Lab Invest. 1997, 77:85-92), these micrographs show less staining for CAP43 in the colon cancer cells compared to the normal cells. Similarly, HIF-1α was found to be higher in normal colon cells (FIG. 13F) compared to colon cancer cells (FIG. 13E).

Many of the same tumor tissues were also stained with antibody that specifically binds CYB1B1, a protein previously reported as a cancer-specific marker (Murray et al., Cancer Res. 1997, 57:3026-303 1). As previously reported by other researchers, however, the protein was found to be expressed at higher levels in normal cells (see, Muskhelishvili et al., J Histochem Cytochem. 2001, 49:229-236). Thus, these data confirm that CAP43 is a superior cancer-specific marker, e.g., when compared to CYB1B1.

The immunohistochemistry experiments in this Example were repeated for identical tissue samples from additional individuals, with consistent results. These data are summarized in Table III, below. For each tissue or cell type examined, the values shown in that table indicate the number of samples expressing which stained positive for either CAP43 of HIF-1α (top number) as a ratio of the total number of samples tested (bottom number). Thus, for example, all of the ten samples of lung cancer tissue examined stained positive for both CAP43 and HIF-1α. All ten samples of healthy lung tissue examined also stained positive for HIF-1α. However, none of the ten samples of healthy lung tissue stained positive for CAP43.

TABLE III
PRESENCE OF CAP43 AND HIF-1α PROTEINS
IN VARIOUS NORMAL AND MALIGNANT TISSUES

| Tissue | CAP43 | HIF-1α |
|---|---|---|
| Normal Lung | 0/10 | 10/10 |
| Lung cancer | 10/10 | 10/10 |
| Normal liver | 0/3 | 0/3 |
| Liver cancer | 3/3 | n.d |
| Normal breast | 0/6 | 0/6 |
| Breast cancer | 4/4 | 2/4 |
| Lymphocytes | 0/3 | 0/3 |
| Smooth muscle | 0/30 | 10/30 |
| Smooth muscle tumor | 4/6 | n.d. |
| Normal brain | 0/3 | 0/3 |
| Brain cancer astrocytomas | 7/7 | 7/7 |
| Brain cancer hemangioblastoma | 3/3 | 3/3 |
| Renal cancer | 22/22 | 0/3 |

These data confirm that CAP43 may be used as specific marker to identify cancer cells. Indeed, as shown here, in many instances a user may distinguish cancer cells from normal cells, e.g., by staining with a CAP43-specific antibody.

### 6.4. Example 4

### Enhanced CAP43 Expression in Esophageal Cancer

This example describes experiments that specifically investigate the differential expression of CAP43 in esophageal cancer. Esophageal cancer in humans occurs world wide with a variable geographic distribution and ranks eighth in order of cancer occurrence. More than 90% of esophageal cancers worldwide are squamous cell carcinomas (SCC). The principal precursor lesion of esophageal SCC is epithelial dysplasia. Microscopically, these lesions represent an accumulation of atypical cells with nuclear hyperchromasia, abnormally clumped chromatin and loss of polarity. These tumors frequently present as fungating, ulcerating or infiltrating lesions in the esophageal epithelium. SCC of the esophagus has a multifactorial etiology involving several environmental and/or genetic factors. Current modalities of therapy of this disease offer poor survival and cure rate. However, clinical investigations have shown that primary chemoprevention of this disease is feasible, if potent inhibitors are identified. To that end, it is important to identify surrogate end-point biomarkers for the disease. Some biomarkers that are currently used to monitor efficacy of chemopreventive agents and other therapies against esophageal SCC include nuclear/nucleolar morphometry using computer-assisted imaging analysis of preneoplastic lesions. However, the use of such biomarkers has proven difficult, emphasizing the need to identify new biomarkers of esophageal SCC.

Samples of SSC, epithelial dysplasia and normal epithelial tissues were obtained from the Kaplan Comprehensive Cancer Center (New York University School of Medicine, New York, N.Y.) and prepared as described in the Examples, supra. CAP43 protein expression levels were assayed in immunohistochemistry experiments performed as described in Section 6.3, above. CAP43 mRNA levels were also measured in an mRNAse protection assay performed according to routine methods (see, e.g., Sambrook et al., 1989, supra, particular in Chapter 7 at pages 7.71-7.78; and Ausubel et al., 1994, supra, particularly in Chapter 4 at pages 4.7.1-4.7.8).

CAP43 mRNA was detected at greatly elevated levels in all 30 esophageal SCC samples evaluated, relative to the normal esophageal tissue. Similarly, the expression of CAP43 protein was very low in normal epithelial tissue, but was increased in epithelial dysplasia. In invasive tumors the level of CAP43 protein detected was significantly higher still compared to the dysplastic tissues.

Thus, the data from these experiments show that CAP43 may be used specifically to detect esophageal cancer, as well as for the numerous other types of cancer discussed, supra. Accordingly, the CAP43 polypeptides, nucleic acids and antibodies of this invention may be used in diagnostic methods for identifying individuals having a particular type of cancer, such as esophageal cancer. Alternatively, CAP43 may be used to monitor the progression of such cancers, e.g., during treatment or therapy. The data also demonstrate that CAP43 can also be used to identify precancerous cells or tissues, such as dysplastic cells or tissues. Such cells and tissues, although not cancerous themselves, have the potential to develop into an invasive or malignant cancer and indicate an increased risk for developing an invasive or malignant cancer.

7. References Cited

Numerous references, including patents, patent applications and various publications, are cited and discussed in the description of this invention. The citation and/or discussion of such references is provided merely to clarify the description of the present invention and is not an admission that any such reference is “prior art” to the invention described herein. All references cited and discussed in this specification are incorporated herein by reference in their entirety and to the same extent as if each reference was individually incorporated by reference.

What is claimed is:

1. A method for identifying a diseased cell or tissue, said disease being associated with abnormal CAP43 expression,
which method comprises detecting, in a cell or tissue, an elevated level of a CAP43 gene product.

2. A method according to claim 1 wherein the CAP43 gene product is encoded by:
(a) a nucleic acid having the nucleotide sequence set forth in FIG. 1A (SEQ ID NO: 1);
(b) a nucleic acid that hybridizes to the complement of the nucleotide sequence set forth in FIG. 1A (SEQ ID NO: 1); or
(c) a nucleic acid at least 70% identical, at the nucleotide level, to the nucleotide sequence set forth in FIG. 1A (SEQ ID NO: 1).

3. A method according to claim 1 wherein the CAP43 gene product is a polypeptide comprising:
(a) the amino acid sequence set forth in FIG. 1B (SEQ ID NO: 2); or
(b) an amino acid sequence at least 70% identical to the sequence set forth in FIG. 1B (SEQ ID NO: 2).

4. A method according to claim 1 wherein the CAP43 gene product is detected by an antibody that specifically binds to a CAP43 polypeptide.

5. A method according to claim 4 wherein the antibody is detectably labeled.

6. A method according to claim 4, which method comprises steps of:
(a) applying the antibody to a cell or tissue; and
(b) detecting binding of the antibody to a CAP43 polypeptide.

7. A method according to claim 6 wherein the antibody is applied in situ to the cell or tissue.

8. A method according to claim 6 wherein the antibody is applied in vivo to the cell or tissue.

9. A method according to claim 1 wherein the diseased cell or tissue is a cancer cell or tissue.

10. A method according to claim 9 wherein the cancer is a lung cancer, a colon cancer, a kidney cancer, a breast cancer, a prostate cancer, a melanoma, a lymphoma, or a malignant fibrous histocytoma.

11. A method according to claim 1 wherein the diseased cell or tissue is granuloma cell or tissue.

12. A method according to claim 1 wherein the diseased cell or tissue is atherosclerotic cell or tissue.

13. A method for identifying a disease cell or tissue, said diseased being associated with abnormal CAP43 expression,
which method comprises detecting, in a cell or tissue, an elevated level of a CAP43 nucleic acid.

14. A method according to claim 13 wherein the CAP43 nucleic acid is:
(a) a nucleic acid having a nucleotide sequence that encodes the amino acid sequence set forth in FIG. 1B (SEQ ID NO: 2);
(b) a nucleic acid that hybridizes to the complement of a nucleotide sequence that encodes the amino acid sequence set forth in FIG. 1B (SEQ ID NO: 2); or
(c) a nucleic acid that encodes an amino acid sequence at least 70% identical to the sequence set forth in FIG. 1B (SEQ ID NO: 2).

15. A method according to claim 14 wherein the CAP43 nucleic acid is:
(a) a nucleic acid having the nucleotide sequence set forth in FIG. 1A (SEQ ID NO: 1);
(b) a nucleic acid that hybridizes to the complement of the nucleotide sequence set forth in FIG. 1A (SEQ ID NO: 1); or
(c) a nucleic acid having a nucleotide sequence at least 70% identical to the nucleotide sequence set forth in FIG. 1A (SEQ ID NO: 1).

16. A method according to claim 13 wherein the CAP43 nucleic acid is detected by a second nucleic acid that specifically hybridizes to the CAP43 nucleic acid.

17. A method according to claim 16 wherein the second nucleic acid is detectably labeled.

18. A method according to claim 16, which method comprises steps of:
(a) contacting nucleic acid from a cell or tissue with the second nucleic acid under conditions suitable for hybridization of the second nucleic acid to CAP43 nucleic acid; and
(b) detecting hybridization of the second nucleic acid to a CAP43 nucleic acid.

19. A method according to claim 18 wherein the second nucleic acid is contacted in situ to nucleic acid from the cell or tissue.

20. A method according to claim 18 wherein the second nucleic acid is contacted in vivo to nucleic acid from the cell or tissue.

21. A method according to claim 13 wherein the diseased cell or tissue is a cancer cell or tissue.

22. A method according to claim 21 wherein the cancer is a lung cancer, a colon cancer, a kidney cancer, a breast cancer, a prostate cancer, a melanoma, a lymphoma, or a malignant fibrous histocytoma.

23. A method according to claim 13 wherein the diseased cell or tissue is granuloma cell or tissue.

24. A method according to claim 13 wherein the diseased cell or tissue is atheroscerotic cell or tissue.

25. A method for diagnosing, in an individual, a disease associated with abnormal CAP43 expression,
which method comprises detecting, in a sample from the individual, an elevated level of a CAP43 gene product.

26. A method according to claim 25 wherein the CAP43 gene product is encoded by:
(a) a nucleic acid having the nucleotide sequence set forth in FIG. 1A (SEQ ID NO: 1);
(b) a nucleic acid that hybridizes to the complement of the nucleotide sequence set forth in FIG. 1A (SEQ ID NO: 1); or
(c) a nucleic acid having a nucleotide sequence at least 70% identical to the nucleotide sequence set forth in FIG. 1A (SEQ ID NO: 1).

27. A method according to claim 25 wherein the CAP43 gene product is a polypeptide comprising:
(a) the amino acid sequence set forth in FIG. 1B (SEQ ID NO: 2); or
(b) an amino acid sequence at least 70% identical to the sequence set forth in FIG. 1B (SEQ ID NO: 2).

28. A method according to claim 25 wherein the gene product is detected by an antibody that specifically binds to a CAP43 polypeptide.

29. A method according to claim 28 wherein the antibody is detectably labeled.

30. A method according to claim 28, which method comprises steps of:
(a) applying the antibody to the sample; and
(b) detecting binding of the antibody to a CAP43 polypeptide.

31. A method according to claim 25 wherein the sample is a body fluid sample.

32. A method according to claim 31 wherein the body fluid sample is a blood sample.

33. A method according to claim 25 wherein the sample is a cell or tissue sample.

34. A method according to claim 25 wherein the disease is cancer.

35. A method according to claim 34 wherein the cancer is a lung cancer, a colon cancer, a kidney cancer, a breast cancer, a prostate cancer, melanoma, a lymphoma or a malignant fibrous histocytoma.

36. A method according to claim 25 wherein the disease is athersclerosis.

37. A method according to claim 25 wherein the disease is granuloma.

38. A method for diagnosing, in an individual, a disease associated with abnormal CAP43 expression,
which method comprises detecting, in a sample from the individual, an elevated level of a CAP43 nucleic acid.

39. A method according to claim 38 wherein the CAP43 nucleic acid is:
(a) a nucleic acid having a nucleotide sequence that encodes the amino acid sequence set forth in FIG. 1B S(EQ ID NO: 2);
(b) a nucleic acid that hybridizes to the complement of a nucleotide sequence that encodes the amino acid sequence set forth in FIG. 1B (SEQ ID NO: 2); or
(c) a nucleic acid having a nucleotide sequence that encodes an amino acid sequence at least 70% identical to the sequence set forth in FIG. 1B (SEQ ID NO: 2).

40. A method according to claim 39 wherein the CAP43 nucleic acid is:
(a) a nucleic acid having the nucleotide sequence set forth in FIG. 1A (SEQ ID NO: 1);
(b) a nucleic acid that hybridizes to the complement of the nucleotide sequence set forth in FIG. 1A (SEQ ID NO: 1); or
(c) a nucleic acid having a nucleotide sequence at least 70% identical to the sequence set forth in FIG. 1A (SEQ ID NO: 1).

41. A method according to claim 38 wherein the CAP43 nucleic acid is detected by a second nucleic acid that specifically hybridizes to the CAP43 nucleic acid.

42. A method according to claim 41 wherein the second nucleic acid is detectably labeled.

43. A method according to claim 41, which method comprises steps of:
(a) contacting nucleic acid from the sample with the second nucleic acid under conditions suitable for hybridization of the second nucleic acid to CAP43 nucleic acid; and
(b) detecting hybridization of the second nucleic acid to CAP43 nucleic acid.

44. A method according to claim 38 wherein the sample is a body fluid sample.

45. A method according to claim 44 wherein the body fluid sample is a blood sample.

46. A method according to claim 38 wherein the sample is a cell or tissue sample.

47. A method according to claim 38 wherein the disease is cancer.

48. A method according to claim 47 wherein the cancer is a lung cancer, a colon cancer, a kidney cancer, a breast cancer, a prostate cancer, melanoma, a lymphoma or a malignant fibrous histocytoma.

49. A method according to claim 38 wherein the disease is atherosclerosis.

50. A method according to claim 38 wherein the disease is granuloma.

51. A method for identifying a cancer cell or tissue, which method comprises detecting, in a cell or tissue, an elevated level of a CAP43 gene product,
wherein the CAP43 gene product has an amino acid sequence:
(a) encoded by a nucleic acid having the nucleotide sequence set forth in FIG. 1A (SEQ ID NO: 1);
(b) encoded by a nucleic acid that hybridizes to the complement of the nucleotide sequence set forth in FIG. 1A (SEQ ID NO: 1);
(c) encoded by a nucleic acid having a nucleotide sequence at least 70% identical to the sequence set forth in FIG. 1A (SEQ ID NO: 1);
(d) comprising the amino acid sequence set forth in FIG. 1B (SEQ ID NO: 2); or
(e) comprising an amino acid sequence at least 70% identical to the sequence set forth in FIG. 1B (SEQ ID NO: 2).

52. A method according to claim 51 wherein the cancer is a lung cancer, a colon cancer, a kidney cancer, a breast cancer, a prostate cancer, a melanoma, a lymphoma, or a malignant fibrous histocytoma.

53. A method for identifying a cancer cell or tissue, which method comprises detecting, in a cell or tissue, an elevated level of a CAP43 nucleic acid,
wherein the CAP43 nucleic acid is:
(a) a nucleic acid having a nucleotide sequence that encodes the amino acid sequence set forth in FIG. 1B (SEQ ID NO: 2);
(b) a nucleic acid that hybridizes to the complement of a nucleotide sequence that encodes the amino acid sequence set forth in FIG. 1B (SEQ ID NO: 2);
(c) a nucleic acid having a nucleotide sequence that encodes an amino acid sequence at least 70% identical to the sequence set forth in FIG. 1B (SEQ ID NO: 2);
(d) a nucleic acid comprising the nucleotide sequence set forth in FIG. 1A (SEQ ID NO: 1);
(e) a nucleic acid that hybridizes to the complement of the nucleotide sequence set forth in FIG. 1A (SEQ ID NO: 1); or
(f) a nucleic acid comprising a nucleotide sequence at least 70% identical to the sequence set forth in FIG. 1A (SEQ ID NO: 1).

54. A method according to claim 53 wherein the cancer is a lung cancer, a colon cancer, a kidney cancer, a breast cancer, a prostate cancer, a melanoma, a lymphoma, or a malignant fibrous histocytoma.

55. A method for diagnosing a cancer in an individual, which method comprises detecting, in a sample from the individual, an elevated level of a CAP43 gene product,
wherein the CAP43 gene product has an amino acid sequence:
(a) encoded by a nucleic acid having the nucleotide sequence set forth in FIG. 1A (SEQ ID NO: 1);
(b) encoded by a nucleic acid that hybridizes to the complement of the nucleotide sequence set forth in FIG. 1A (SEQ ID NO: 1);
(c) encoded by a nucleic acid having a nucleotide sequence at least 70% identical to the sequence set forth in FIG. 1A (SEQ ID NO:l);
(d) comprising the amino acid sequence set forth in FIG. 1B (SEQ ID NO: 2); or
(e) comprising an amino acid sequence at least 70% identical to the sequence set forth in FIG. 1B (SEQ ID NO: 2).

56. A method according to claim 55 wherein the cancer is a lung cancer, a colon cancer, a kidney cancer, a breast cancer, a prostate cancer, a melanoma, a lymphoma, or a malignant fibrous histocytoma.

57. A method for diagnosing a cancer in an individual, which method comprises detecting, in a sample from the individual, an elevated level of a CAP43 nucleic acid,
wherein the CAP43 nucleic acid is:
(a) a nucleic acid having a nucleotide sequence that encodes the amino acid sequence set forth in FIG. 1B (SEQ ID NO: 2);
(b) a nucleic acid that hybridizes to the complement of a nucleotide sequence that encodes the amino acid sequence set forth in FIG. 1B (SEQ ID NO: 2);
(c) a nucleic acid having a nucleotide sequence that encodes an amino acid sequence at least 70% identical to the sequence set forth in FIG. 1B (SEQ ID NO: 2);
(d) a nucleic acid comprising the nucleotide sequence set forth in FIG. 1A (SEQ ID NO: 1);
(e) a nucleic acid that hybridizes to the complement of the nucleotide sequence set forth in FIG. 1A (SEQ ID NO: 1); or
(f) a nucleic acid comprising a nucleotide sequence at least 70% identical to the sequence set forth in FIG. 1A (SEQ ID NO: 1).

58. A method according to claim 58, wherein the cancer is a lung cancer, a colon cancer, a kidney cancer, a breast cancer, a prostate cancer, a melanoma, a lymphoma, or a malignant fibrous histocytoma.

59. A method for administering a compound to a diseased cell, said disease being associated with abnormal CAP43 expression,
which method comprises contacting the cell with the compound complexed to a protein that specifically binds to a CAP43 polypeptide.

60. A method according to claim 59 wherein the CAP43 polypeptide has an amino acid sequence:
(a) encoded by a nucleic acid having the nucleotide sequence set forth in FIG. 1A (SEQ ID NO: 1);
(b) encoded by a nucleic acid that hybridizes to the complement of the nucleotide sequence set forth in FIG. 1A (SEQ ID NO: 1);
(c) encoded by a nucleic acid having a nucleotide sequence at least 70% identical to the sequence set forth in FIG. 1A (SEQ ID NO: 1);
(d) comprising the amino acid sequence set forth in FIG. 1B (SEQ ID NO: 2); or
(e) comprising an amino acid sequence at least 70% identical to the sequence set forth in FIG. 1B (SEQ ID NO: 2).

61. A method according to claim 59 wherein the polypeptide that specifically binds to a CAP43 polypeptide is an antibody.

62. A method according to claim 59 wherein the compound is a toxin.

63. A method according to claim 62 wherein the toxin is a thymidine kinase, an endonuclease, an RNAse, an α-toxin, ricin, abrin, an exotoxin A, a diphtheria toxin, saporin, momordin, gelonin, a pokeweed antiviral protein, α-sarcin, or a cholera toxin.

64. A method according to claim 59 wherein the compound is a cytoxin.

65. A method according to claim 64 wherein the cytotoxin is a benzoic acid mustard alkylating agent derivative, a etoposide derivative, a mitomycin C derivative , or a doxorubicin derivative.

66. A method according to claim 65 wherein the cytotoxin is a glutamyl derivative of a benzoic acid mustard alkylating agent.

67. A method according to claim 65 wherein the cytotoxin is a phosphate derivative or etoposide.

68. A method according to claim 65 wherein the cytotoxin is a phosphate derivative of mitomycin C.

69. A method according to claim 65 wherein the cytotoxin is a phenoxyacetamide derivative of doxorubicin.

70. A method according to claim 59 wherein the diseased cell is a cancer cell.

71. A method according to claim 70 wherein the cancer is a lung cancer, a kidney cancer, abreast cancer, a prostate cancer, a melanoma, a lymphoma or a malignant fibrous histocytoma.

72. A method according to claim 59 wherein the diseased cell is an atherosclerotic cell.

73. A method according to claim 59 wherein the cell is a granuloma cell.

74. A complex comprising:
(a) an antibody that specifically binds to a CAP43 polypeptide; and
(b) a therapeutic compound.

75. A complex according to claim 74 wherein the CAP43 polypeptide has an amhino acid sequence:
(a) encoded by a nucleic acid having the nucleotide sequence set forth in FIG. 1A (SEQ ID NO: 1);
(b) encoded by a nucleic acid that hybridizes to the complement of the nucleotide sequence set forth in FIG. 1A (SEQ ID NO: 1);
(c) encoded by a nucleic acid having a nucleotide sequence at least 70% identical to the sequence set forth in FIG. 1A (SEQ ID NO: 1);
(d) comprising the amino acid sequence set forth in FIG. 1B (SEQ ID NO: 2); or
(e) comprising an amino acid sequence at least 70% identical to the sequence set forth in FIG. 1B (SEQ ID NO: 2).

76. A complex according to claim 74 wherein the therapeutic compound is a drug, a pro-drug, a toxin or a cytotoxin.

77. A complex according to claim 76 wherein the therapeutic compound is a toxin selected from the group consisting of a thymidine kinase, an endonuclease, an RNAse, an α-toxin, ricin, abrin, an exotoxin A, a diphtheria toxin, saporin, momordin, gelonin, a pokeweed antiviral protein, α-sarcin, and a cholera toxin.

78. A complex according to claim 76 wherein the therapeutic compound is a cytotoxin selected from the group consisting of a benzoic acid mustard alkylating agent derivative, a etoposide derivative, a mitomycin C derivative , and a doxorubicin derivative.

79. A complex according to claim 74 wherein the therpeutic compound is covalently attached to the antibody.

80. A complex according to claim 74 wherein the antibody comprises:
(a ) a first Fab′ arm that specifically binds to a CAP43 polypeptide; and
(b) a second Fab′ arm that specifically binds to the therapeutic compound.

81. A complex according to claim 74 which further comprises a polypeptide having:
(i) a first binding domain that binds to the antibody; and
(ii) a second binding domain that binds to the therapeutic compound.

82. A pharmaceutical composition comprising:
(a) an antibody that specifically binds to a CAP43 polypeptide and has a therapeutic compound attached thereto; and
(b) a pharmaceutically acceptable carrier.

83. A pharmaceutical composition according to claim 82 in which the CAP43 polypeptide has an amino acid sequence:
(a) encoded by a nucleic acid having the nucleotide sequence set forth in FIG. 1A (SEQ ID NO: 1);
(b) encoded by a nucleic acid that hybridizes to the complement of the nucleotide sequence set forth in FIG. 1A (SEQ ID NO: 1);
(c) encoded by a nucleic acid having a nucleotide sequence at least 70% identical to the sequence set forth in FIG. 1A (SEQ ID NO: 1);
(d) comprising the amino acid sequence set forth in FIG. 1B (SEQ ID NO: 2); or
(e) comprising an amino acid sequence at least 70% identical to the sequence set forth in FIG. 1B (SEQ ID NO: 2).

84. A kit for identifying a diseased cell or tissue, said disease being associated with abnormal CAP43 expression, which kit comprises:
(a) at least on of (i) a nucleic acid that specifically hybridizes to a CAP43 nucleic acid, or (ii) an antibody that specifically binds to a CAP 43 polypeptide; and
(b) instructions for using said kit.

85. A kit according to claim 84, wherein the CAP43 nucleic acid comprises:
(a) a nucleotide sequence that encodes that amino acid sequence set forth in FIG. 1B (SEQ ID NO: 2);
(b) a nucleic acid that hybridizes to the complement of a nucleotide sequence that encodes the polypeptide set forth in FIG. 1B (SEQ ID NO: 2);
(c) a nucleotide sequence that encodes an amino acid sequence at least 70% identical to the sequence set forth in FIG. 1B (SEQ ID NO: 2);
(d) the nucleotide sequence set forth in FIG. 1A (SEQ ID NO: 1);
(e) a nucleic acid that hybridizes to the complement of the nucleotide sequence set forth in FIG. 1A (SEQ ID NO: 1); or
(f) a nucleotide sequence at least 70% identical to the sequence set forth in FIG. 1A (SEQ ID NO: 1).

86. A kit according to claim 84 wherein the CAP43 polypeptide has an amino acid sequence:
(a) encoded by a nucleic acid having the nucleotide sequence set forth in FIG. 1A (SEQ ID NO: 1);
(b) encoded by a nucleic acid that hybridizes to the complement of the nucleotide sequence set forth in FIG. 1A (SEQ ID NO: 1); or
(c) comprising the amino acid sequence set forth in SEQ ID NO: 2.

87. A kit according to claim 84 wherein the disease cell or tissue is a cancer cell or tissue.

88. A kit according to claim 87 wherein the cancer is a lung cancer, a colon cancer, a kidney cancer, a breast cancer, a prostate cancer, a melanoma, a lymphoma or a malignant fibrous histocytoma.

89. A method for treating a disorder associated with abnormal CAP43 expression or activity, which method comprises contacting a cell with a compound that inhibits expression or activity of a CAP43 nucleic acid so that one or more symptoms of the disorder are ameliorated.

90. A method according to claim 89 wherein the CAP43 nucleic acid comprises:
(a) a nucleotide sequence that encodes the amino acid sequence set forth in FIG. 1B (SEQ ID NO: 2);
(b) a nucleic acid that hybridizes to the complement of a nucleotide sequence that encodes the polypeptide set forth in FIG. 1B (SEQ ID NO: 2);
(c) a nucleotide sequence that encodes an amino acid sequence at least 70% identical to the sequence set forth in FIG. 1B (SEQ ID NO: 2);
(d) the nucleotide sequence set forth in FIG. 1A (SEQ ID NO: 1);
(e) a nucleic acid that hybridizes to the complement of the nucleotide sequence set forth in FIG. 1A (SEQ ID NO: 1); or
(f) a nucleotide sequence at least 70% identical to the sequence set forth in FIG. 1A (SEQ ID NO: 1).

91. A method according to claim 89 wherein the disorder is a cancer.

92. A method according to claim 91 wherein the cancer is a lung cancer, a colon cancer, a kidney cancer, a breast cancer, a prostate cancer, a melanoma cancer, a lymphoma or a malignant fibrous histocytoma.

93. A method for treating a disorder associated with abnormal CAP43 expression or activity, which method comprises contacting a cell with a compound that inhibits expression or activity of a CAP43 polypeptide.

94. A method according to claim 93 wherein the CAP43 polypeptide has an amino acid sequence:
(a) encoded by a nucleic acid having the nucleotide sequence set forth in FIG. 1A (SEQ ID NO: 1);
(b) encoded by a nucleic acid that hybridizes to the complement of the nucleotide sequence set forth in FIG. 1A (SEQ ID NO: 1);
(c) encoded by a nucleic acid having a nucleotide sequence at least 70% identical to the sequence set forth in FIG. 1A (SEQ ID NO: 1);
(d) comprising the amino acid sequence set forth in FIG. 1B (SEQ ID NO: 2); or
(e) comprising an amino acid sequence at least 70% identical to the sequence set forth in FIG. 1B (SEQ ID NO: 2).

95. A method according to claim 93 wherein the disorder is a cancer.

96. A method according to claim 95 wherein the cancer is a lung cancer, a colon cancer, a kidney cancer, a breast cancer, a prostate cancer, a melanoma, a lymphoma or a malignant fibrous histoma.

97. A pharmaceutical composition for treating cancer,
which pharmaceutical composition comprises a compound that inhibits expression or activity of a CAP43 nucleic acid, and
wherein the compound is present in said pharmaceutical composition in an amount sufficient to ameliorate one or more symptoms of the cancer.

98. A pharmaceutical composition according to claim 97 wherein the CAP43 nucleic acid comprises:
(a) a nucleotide sequence that encodes the amino acid sequence set forth in FIG. 1B (SEQ ID NO: 2);
(b) a nucleic acid that hybridizes to the complement of a nucleotide sequence that encodes the polypeptide set forth in FIG. 1B (SEQ ID NO: 2);
(c) a nucleotide sequence that encodes an amino acid sequence at least 70% identical to the sequence set forth in FIG. 1B (SEQ ID NO: 2);
(d) the nucleotide sequence set forth in FIG. 1A (SEQ ID NO: 1);
(e) a nucleic acid that hybridizes to the complement of the nucleotide sequence set forth in FIG. 1A (SEQ ID NO: 1); or
(f) a nucleotide sequence at least 70% identical to the sequence set forth in FIG. 1A (SEQ ID NO: 1).

99. A pharmaceutical composition according to claim 97 wherein the cancer is a lung cancer, a colon cancer, a kidney cancer, a breast cancer, a prostate cancer, a melanoma cancer, a lymphoma or a malignant fibrous histocytoma.

100. A pharmaceutical composition for treating cancer,
which pharmaceutical composition comprises a compound that inhibits expression or activity of a CAP43 polypeptide, and
wherein the compound is present in said pharmaceutical composition in an amount sufficient to ameliorate one or more symptoms of the cancer.

101. A pharmaceutical composition according to claim 100 wherein the CAP43 polypeptide has an amino acid sequence:
(a) encoded by a nucleic acid having the nucleotide sequence set forth in FIG. 1A (SEQ ID NO: 1);
(b) encoded by a nucleic acid that hybridizes to the complement of the nucleotide sequence set forth in FIG. 1A (SEQ ID NO: 1);
(c) encoded by a nucleic acid having a nucleotide sequence at least 70% identical to the sequence set forth in FIG. 1A (SEQ ID NO: 1);
(d) comprising the amino acid sequence set forth in FIG. 1B (SEQ ID NO: 2); or
(e) comprising an amino acid sequence at least 70% identical to the sequence set forth in FIG. 1B (SEQ ID NO: 2).

102. A pharmaceutical composition according to claim 100 wherein the cancer is a lung cancer, a colon cancer, a kidney cancer, a breast cancer, a prostate cancer, a melanoma, a lymphoma or a malignant fibrous histoma.


SEQUENCE LISTING (3 sequences)

SEQ ID NO: 1 - DNA, 2972 bp, Homo sapiens
ctcgcgttaggcaggtgacagcagggacatgtctcgggagatgcaggatgtagacctcgctgaggtgaagcctttggtggagaaaggggagaccatcaccggcctcctgcaagagtttgatgtccaggagcaggacatcgagactttacatggctctgttcacgtcacgctgtgtgggactcccaagggaaaccggcctgtcatcctcacctaccatgacatcggcatgaaccacaaaacctgctacaaccccctcttcaactacgaggacatgcaggagatcacccagcactttgccgtctgccacgtggacgcccctggccagcaggacggcgcagcctccttccccgcagggtacatgtacccctccatggatcagctggctgaaatgcttcctggagtccttcaacagtttgggctgaaaagcattattggcatgggaacaggagcaggcgcctacatcctaactcgatttgctctaaacaaccctgagatggtggagggccttgtccttatcaacgtgaacccttgtgcggaaggctggatggactgggccgcctccaagatctcaggatggacccaagctctgccggacatggtggtgtcccacctttttgggaaggaagaaatgcagagtaacgtggaagtggtccacacctaccgccagcacattgtgaatgacatgaaccccggcaacctgcacctgttcatcaatgcctacaacagccggcgcgacctggagattgagcgaccaatgccgggaacccacacagtcaccctgcagtgccctgctctgttggtggttggggacagctcgcctgcagtggatgccgtggtggagtgcaactcaaaattggacccaacaaagaccactctcctcaagatggcggactgtggcggcctcccgcagatctcccagccggccaagctcgctgaggccttcaagtacttcgtgcagggcatgggatacatgccctcggctagcatgacccgcctgatgcggtcccgcacagcctctggttccagcgtcacttctctggatggcacccgcagccgctcccacaccagcgagggcacccgaagccgctcccacaccagcgagggcacccgcagccgctcgcacaccagcgagggggcccacctggacatcacccccaactcgggtgctgctgggaacagcgccgggcccaagtccatggaggtctcctgctaggcggcctgcccagctgccgcccccggactctgatctctgtagtggccccctcctccccggccccttttcgccccctgcctgccatactgcgcctaactcggtattaatccaaagcttattttgtaagagtgagctctggtggagacaaatgaggtctattacgtgggtgccctctccaaaggcggggtggcggtggaccaaaggaaggaagcaagcatctccgcatcgcatcctcttccattaaccagtggccggttgccactctcctcccctccctcagagacaccaaactgccaaaaacaagacgcgtagcagcacacacttcacaaagccaagcctaggccgccctgagcatcctggttcaaacgggtgcctggtcagaaggccagccgcccacttcccgtttcctctttaactgaggagaagctgatccagtttccggaaacaaaatccttttctcatttggggaggggggtaatagtgacatgcaggcacctcttttaaacaggcaaaacaggaagggggaaaaggtgggattcatgtcgaggctagaggcatttggaacaacaaatctacgtagttaacttgaagaaaccgatttttaaagttggtgcatctagaaagctttgaatgcagaagcaaacaagcttgatttttctagcatcctcttaatgtgcagcaaaagcaggcgacaaaatctcctggctttacagacaaaaatatttcagcaaacgttgggcatcatggtttttgaaggctttagttctgctttctgcctctcctccacagccccaacctcccacccctgatacatgagccagtgattattcttgttcagggagaagatcatttagatttgttttgcattccttagaatggagggcaacattccacagctgccctggctgtgatgagtgtccttgcaggggccggagtaggagcactggggtgggggtggaattggggttactcgatgtaagggattccttgttgttgtgttgagatccagtgcagttgtgatttctgtggatcccagcttggttccaggaattttgtgtgattggcttaaatccagttttcaatcttcgacagctgggctggaacgtgaactcagtagctgaacctgtctgacccggtcacgttcttggatcctcagaactctttgctcttgtcggggtgggggtgggaactcacgtggggagcggtggctgagaaaatgtaaggattctggaatacatattccatgggactttccttccctctcctgcttcctcttttcctgctccctaacctttcgccgaatggggcagcaccactgacgtttctgggcggccagtgcggctgccaggttcctgtactactgccttgtacttttcattttggctcaccgtggattttctcataggaagtttggtcagagtgaattgaatattgtaagtcagccactgggacccgaggatttttgggaccccgcagttgggaggaggaagtagtccagccttccaggtggcgtgagaggcaatgactcgttacctgccgcccatcaccttggaggccttccctggccttgagtagaaaagtcggggatcggggcaagagaggctgagtacggatgggaaactattgtgcacaagtctttccagaggagtttcttaatgagatatttgtatttatttccagaccaataaatttgtaactttgcaaaaaaaaaaaaaaaaaaaaaaaaaaaaaaaaaaaaaaaaaaaaaaaaaaaaaaaaa

SEQ ID NO: 2 - protein, 394 aa, Homo sapiens
MSREMQDVDLAEVKPLVEKGETITGLLQEFDVQEQDIETLHGSVHVTLCGTPKGNRPVILTYHDIGMNHKTCYNPLFNYEDMQEITQHFAVCHVDAPGQQDGAASFPAGYMYPSMDQLAEMLPGVLQQFGLKSIIGMGTGAGAYILTRFALNNPEMVEGLVLINVNPCAEGWMDWAASKISGWTQALPDMVVSHLFGKEEMQSNVEVVHTYRQHIVNDMNPGNLHLFINAYNSRRDLEIERPMPGTHTVTLQCPALLVVGDSSPAVDAVVECNSKLDPTKTTLLKMADCGGLPQISQPAKLAEAFKYFVQGMGYMPSASMTRLMRSRTASGSSVTSLDGTRSRSHTSEGTRSRSHTSEGTRSRSHTSEGAHLDITPNSGAAGNSAGPKSMEVSC

SEQ ID NO: 3 - DNA, 2131 bp, Homo sapiens
tttcatcttttttgtagagatgggacatcactatgttgtgaaggctggtctcaaactcctggactcaagagaggcttctgcattggcctcccaaagtgctgggattacaggtgtaagccactctacctggacacaacattttatttgaaataagaaaaattttccctcactgggaatgctgcagttgaactagttcaaatccataaagaatggaagactgcccagtggtcattcattcacttattaattctagatgttaaattttctagggtgggggaatgtgctcctacattcaaggcattcttgaatgtagccaaactttgtagtttagccaaacttcatagctaatagccaaactttgtagtttagccaaactcttctagcagcaaaacccatgacactggaactctgttcttcacactggtcatttgtaatagaataacagagctttagcactagataggttctgaaagatcattgaatctaatcctctccctttgccagtgagaattctgaagcccagatttgttgatttgtgatcgatagtgtcaaagacaggcctgaaacacagatgtcctgggtcctagaggtgctgtttgcccctctccatatttcttttgttccagaaaacccttctccaaaactggccctaataatcagaggggaaagccatggcccctgccttggggacagcatgggttggcacagaaaagaggtttacaattcagcaggaagtgttgtgcgtgcgcgcgtgtgtgtctgtggaggcgcagggagggtcacctgagcttgccctgggtctggtcctgggctcagtggcaaattcaacgctgggcaggtggcctgaggacaatgacggtgccagctgtgagtcagacttcctttattcataaaatcatgttcctccatgcagtgcttctcaacctggctgcacgtcaggagccacctgggtagctttgtaaaaaccccaatgcccagttggtttgcaccccctgagattctgattaaactgttctggagtccatctctctctttttattatctatctatttatttattttttaatttggcggctcctctgggacttctcatatgcagtaggggttgagaattgctggtttctttggccagatgggaacatgcaggccaatagttacgcacaaacagagcagcagagcaggacggtgctaaggttggaaagggtgagtcatgaaagttcaggaaatcagaataggaaggggtgagaatcctctcatctgggcttgtggggcaggagagaagagcccagtgggcaaggtccagtgagtgtgcttagaactgggaagatgaggggaagggtaagcgctgggcggcggtgggcaatatggacgagattaaacgggagcgaggatttcagcgagagctttggcgacacggaaggctgagaatcggagggagcctgtcgggcgaaagcgaaggcggctgctgggcaggcagccgaatccggctggagagccgagctggtgagacctacaggaagtgaagggagtcgctcagggcgtggcgcaacgagactcttagaagaaactctgaggcagagatggggggcctccgcccatacggagacacaaggaagtccacatgcacacgcacgagcgcgcacatgaacacgcacaagcacacaaacgcctcctccgggcagggcacacgcgcccgctgcacaggccgaggccctggactcggaggggactgcagagccgacccacaacccgggccccgatgcccctcccggccgcgcccctacgactgcttgcgcaacaggcggcggctccagtgggcgcccgccgcgcgctgccggagcccagcccagcccggcgcgcccgggaggagggagcagggagcggggaaggggtgtgtcccggctgcgtgctgggactgcgagggtctgggaggggcgaggcgcgggggcggggccgcggcgcctataaagtcgccctccgcccggacgtaaacaaacctcgcctggctcccagctggtgctgaagctcgtcagttcaccatccgccctcggcttccgcggggcgctgggccgccagcctcggcaccgtcctttcctttctccctcgcgttaggcaggtgacagcagggcatg